SEQUENCE LISTING (24 sequences)

SEQ ID NO: 1 - DNA, 5102 bp, Homo sapiens
gagcggccgcgccgggtccccgggacggggtggaagtgggggtggggggaggggatcggggccgggccggggccgcgctgcctgcgatgccgggcgcccgccgcagccgctgccgccggagcccgggatggggcgagaggctgcggcggacgccagcatctccccgccggggaccccgggggccgcggagccgccgccgccgctgctgccgccgttgctgagacccagcgggcgatgggatgaagatgagacgctacaagctctttctcatgttctgtatggccggcctgtgcctcatctccttcctgcacttcttcaagaccctgtcctatgtcaccttcccccgagaactggcctccctcagccctaacctggtgtccagctttttctggaacaatgccccggtcacgccccaggccagccccgagccaggaggccctgacctgctgcgtaccccactctactcccactcgcccctgctgcagccgctgccgcccagcaaggcggccgaggagctccaccgggtggacttggtgctgcccgaggacaccaccgagtatttcgtgcgcaccaaggccggcggcgtctgcttcaaacccggcaccaagatgctggagaggccgcccccgggacggccggaggagaagcctgagggggccaacggctcctcggcccggcggccaccccggtacctcctgagcgcccgggagcgcacggggggccgaggcgcccggcgcaagtgggtggagtgcgtgtgcctgcccggctggcacggacccagctgcggcgtgcccactgtggtgcagtactccaacctgcccaccaaggagcggctggtgcccagggaggtgccgcgccgcgtcatcaacgccatcaacgtcaaccacgagttcgacctgctggacgtgcgcttccacgagctgggcgacgtggtggacgcctttgtggtgtgcgagtccaacttcacggcttatggggagccgcggccgctcaagttccgggagatgctgaccaatggcaccttcgagtacatccgccacaaggtgctctatgtcttcctggaccacttcccgcccggcggccggcaggacggctggatcgccgacgactacctgcgcaccttcctcacccaggacggcgtctcgcggctgcgcaacctgcggcccgacgacgtcttcatcattgacgatgcggacgagatcccggcccgtgacggcgtccttttcctcaagctctacgatggctggaccgagcccttcgccttccacatgcgcaagtcgctctacggcttcttctggaagcagccgggcaccctggaggtggtgtcaggctgcacggtggacatgctgcaggcagtgtatgggctggacggcatccgcctgcgccgccgccagtactacaccatgcccaacttcagacagtatgagaaccgcaccggccacatcctggtgcagtggtcgctgggcagccccctgcacttcgccggctggcactgctcctggtgcttcacgcccgagggcatctacttcaagctcgtgtccgcccagaatggcgacttcccacgctggggtgactacgaggacaagcgggacctgaactacatccgcggcctgatccgcaccgggggctggttcgacggcacgcagcaggagtacccgcctgcagaccccagcgagcacatgtatgcgcccaagtacctgctgaagaactacgaccggttccactacctgctggacaacccctaccaggagcccaggagcacggcggcgggcgggtggcgccacaggggtcccgagggaaggccgcccgcccggggcaaactggacgaggcggaagtctagagctgcatgatctgatagggtttgtgacagggcgggggtggcggcggcccctagcgctatctccctgcctcctgccggctccttggttcttgaggggaccaggagtgggtggggagtgggggtgggggtagggtttccctactgaagcccttgtgaatcaagggtcaggcctttgagctcagaaaatatccctcctgttgggagagggcgcaggccgtgacgtctgggtggcccttatgactgccaagactgctgtggccaggaggtgccactggagtgtgcgtggtggtccctgggtagcgggggagggtaggcaggattggggaagagagcctgcaggatctcaccaggcagcctctggggggtggccaggccgggaaaaagcccaccatttggcatccctgggccttgggctccgtgtgggagaccggcctgccaggaggacccagggctctgtaagtagatgcatttgggtccaggaggaagcgtggacacctcgtagggaagagatgaaaaagccacatcctaccaagaggaggtgctgagggatgctttgcagtgtagtcagaagtgctgggccagatggagacagaactccaccccctgccgcaaaggacaggacctggctgccctgggatgctggtgcctgagtctgtctctgtgcacccctcaggctgtcgtgagccaacacaggggcctggagaaccctgaggagctttccttttggttctaaacccggcgttgacgttccttctccctttcacattgctgtcttgtggactgtgcactcagtccttgcaaggccaagagtccagttgtaggtgtggccttgagggggaagtggggaggagaagactgacatgagtcctctgcacggatccgtctctccctccccatcaccccttccttctgacacccagtcccagctgtccactgtcccaggtgcagtcactgttgtgcccttccttggggcaggctggctgggggccagaaaggggccatgaggctgtcttgggcccaaaaagggacaataaggccagttgtatgcttcctgttcctcatagcttgccttggtggggatgtctttgttggagttgattctgagctgctgtgattaggagaccctgaaatacagtggtttaagcaagatggaagcttgtttctaattagtctagattgagatggcccagagctggtagggcagctctgcgtttcttcatacgcaccttccaattctgggtacacagcggctgctccagcgcccaccctcctgtgtgcatccaagcctgggggaagcagaaatagacaagagggcacacccactttttgctaaaggcatgagccagaattggcaggctcacctctgctggcctctcattggctgggactcagtcacatggccacaagcagctgctagggaacctgggaagtgtagtcttcagcggggccgccatgtgcctggcctcaccttgggagttatcttattgatggaggagaagagaatggatatgggggaccagtagcatctctgggagagggggagggagcagcaataactcagtcgtcggatccagctctcattgtcagagtttccggaacagcttgctcctgtttccctcactgtgcagcccagggctgggggcagtgaggagcttgcagctctgtgggaaggggaaacaccccctcccctcggcccctcagacgctacccaatgatgccggtttgcagagttggcctgtggaatggctcatgtttgtgcgtgtgtgtgtgtatatttatgggcatgggtgcatgcttggtgtgtatttgtacatgtctgtattgctgtgtccctgtaaatacatgcttgtgtatggatggaagaggccaggcccaggcctggcctcttcctcgggcctgtggccacacctcctgcagctccccaaaatgactgaggcagaaagcccttggggagcctagaaagcaaagctaaaggggatgcagggtctgtctgtctgtctgtctttcagtctgaggaatgagaatcctgacctgagggctgtgcagctgagagcccactacctccccagcccctctcggccccagccgcatcatcccacctgtcccctcccccccacctccagtggggctttctccagatgtcttatggttgggggtttcctgatgggccaggagaggagggcatcttcttgcgacagcactgtctgggttaagtgcccagtgagggcatggtgtggggagctggcctcagaggagccgctggtgggcaagcgtgaagtgggctgaggggctctgagccactttgctcccatctaggggactgccccccatggaactcctttgaagtcacagcagccttcctttctgtttgctcttggggctgagaggtggctcaaacactcggggtccctatggctctgggtcaatctaggccaggctgcaccccatggacagggagtctcagggctcctgatcatgcccaggccctggcctggggcctccctccttggcagctttcccacccccacgcccctggcatcctcagttgctatgggatgcccctccagggcaccagctcagggctaagcgaaggaagataggagcagctcagagctgccaggctctgccttcctcacagacctggtggggcaggtcctgttcacagcagcaggagtgaaggcctggccatcggtggagagggcagctgtcagagggctgggggccagggcacaggattgaagagtttcacatatcatcacagcatacactgggaatttggtgggggcagaagaacccagggccactccctcaatatgaagggaaaccaagctgaatgtgaccaccggcacactgctgccatgtcccatgtccacctttctccccgggaataactggccctgagacccctagacccaaggaggcctgtccatgccaagcatccgggaagcatggctggccttatccacccatgggtcacgtcggttcccaggggcagcatgggagatctttgggggcaacagggagagtctgggtggggagacgggacttgtccaagcagaaggcaggaccctgggaaatgcataatgtaaggacatcaataatagtattattttttttgtaagggaaaatcaatatgtacattctgaaatcattttctctgtaaatggttggatttcatttcacccttaaagggatgcttaaaggagaagataatattaataataaaaacagctacaaagtctgaaaaaaaaaaaaaaaaaa

SEQ ID NO: 2 - DNA, 3057 bp, Homo sapiens
cactttcgagagtgccgtctatttgccacacacttccctgatgaaatgtctggatttggactaaagaaaaaaggaaaggctagcagtcatccaacagaatcatgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaaatttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgtaaaaaaaaaaaaaaaaaa

SEQ ID NO: 3 - DNA, 5503 bp, Homo sapiens
tttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaactacgtgtgaaggtattcaaggcagggagtatacattgctgtttcctgttgggcaatgctccttgaccacattttgggaagagtggatgttatcattgagaaaacaatgtgtctggaattaatggggttcttataaagaaggttcccagaaaagaatgttcatccagcctcctcagaaacagaacattcaagaaaaggacaatcaggatgtcatcagggaaatgaaaataaaaaccacaatgagatatcaccttataccaggtagaatggctactataaaaaaatgaagtgtcatcaaggatatagagaaattggaacccttcttcactgctggagggaatggaaaatggtgtagccgttatgaaaaacagtacggaggtttctcaaaaattaaaaatagaactgctatatgatccagcaatctcacttctgtatatatacccaaaataattgaaatcagaatttcaagaaaatatttacactcccatgttcattgtggcactcttcacaatcactgtttccaaagttatggaaacaacccaaatttccattgaaaaataaatggacaaagaaaatgtgcatatacgtacaatgggatattattcagcctaaaaaaagggggaatcctgttatttatgacaacatgaataaacccggaggccattatgctatgtaaaatgagcaagtaacagaaagacaaatactgcctgatttcatttatatgaggttctaaaatagtcaaactcatagaagcagagaatagaacagtggttcctagggaaaaggaggaagggagaaatgaggaaatagggagttgtctaattggtataaaattatagtatgcaagatgaattagctctaaagatcagctgtatagcagagttcgtataatgaacaatactgtattatgcacttaacattttgttaagagggtacctctcatgttaagtgttcttaccatatacatatacacaaggaagcttttggaggtgatggatatatttattaccttgattgtggtgatggtttgacaggtatgtgactatgtctaaactcatcaaattgtatacattaaatatatgcagttttataatatcaattatgtctgaatgaagctataaaaaagaaaagacaacaaaattcagttgtcaaaactggaaatatgaccacagtcagaagtgtttgttactgagtgtttcagagtgtgtttggtttgagcaggtctagggtgattgaacatccctgggtgtgtttccatgtctcatgtactagtgaaagtagatgtgtgcatttgtgcacatatccctatgtatccctatcagggctgtgtgtatttgaaagtgtgtgtgtccgcatgatcatatctgtatagaagagagtgtgattatatttcttgaagaatacatccatttgaaatggatgtctatggctgtttgagatgagttctctactcttgtgcttgtacagtagtctccccttatcccttatgcttggtggatacgttcttagaccccaagtggatctctgagaccgcagatggtaccaaacctcatatatgcaatattttttcctatacataaatacctaagataaagttcatcttctgaattaggcacagtaagagattaacaataactaacaataaaattgaatagttataataatatattgtaataaaagttatgtgaatgtgatctctttctttctctctctcaaaa

SEQ ID NO: 4 - DNA, 3431 bp, Homo sapiens
ggttttcaggagcccgagcgagggcgccgcttttgcgtccgggaggagccaaccgtggcgcaggcggcgcggggaggcgtcccagagtctcactctgccgcccaggctggactgcagtgacacaatctcggctgactgcaaccactgcctccagggttcaagcgattctcttgcctcagcctcccaagtagctgggattacagattgatgttcatgttcctgacactactacaagattcatactcctgatgctactgacaacgtggcttctccacagtcaccaaaccagggatgctatactggacttccctactctcatctgctccagccccctgaccttatagttgcccagctttcctggcaattgactttgcccatcaatacacaggatttagcatccagggaagatgtcggagcctcagatgttaattttctaattgagaatgttggcgctgtccgaacctggagacaggaaaacaaaaagtcctttctcctgattcaccaaaaaataaaatactgactaccatcactgtgatgagattcctatagtctcaggaactgaagtctttaaacaaccagggaccctctgcccctagaataagaacatactagaagtcccttctgctaggacaacgaggatcatgggagaccacctggaccttctcctaggagtggtgctcatggccggtcctgtgtttggaattccttcctgctcctttgatggccgaatagccttttatcgtttctgcaacctcacccaggtcccccaggtcctcaacaccactgagaggctcctgctgagcttcaactatatcaggacagtcactgcttcatccttcccctttctggaacagctgcagctgctggagctcgggagccagtatacccccttgactattgacaaggaggccttcagaaacctgcccaaccttagaatcttggacctgggaagtagtaagatatacttcttgcatccagatgcttttcagggactgttccatctgtttgaacttagactgtatttctgtggtctctctgatgctgtattgaaagatggttatttcagaaatttaaaggctttaactcgcttggatctatccaaaaatcagattcgtagcctttaccttcatccttcatttgggaagttgaattccttaaagtccatagatttttcctccaaccaaatattccttgtatgtgaacatgagctcgagcccctacaagggaaaacgctctccttttttagcctcgcagctaatagcttgtatagcagagtctcagtggactggggaaaatgtatgaacccattcagaaacatggtgctggagatactagatgtttctggaaatggctggacagtggacatcacaggaaactttagcaatgccatcagcaaaagccaggccttctctttgattcttgcccaccacatcatgggtgccgggtttggcttccataacatcaaagatcctgaccagaacacatttgctggcctggccagaagttcagtgagacacctggatctttcacatgggtttgtcttctccctgaactcacgagtctttgagacactcaaggatttgaaggttctgaaccttgcctacaacaagataaataagattgcagatgaagcattttacggacttgacaacctccaagttctcaatttgtcatataaccttctgggggaactttacagttcgaatttctatggactacctaaggtagcctacattgatttgcaaaagaatcacattgcaataattcaagaccaaacattcaaattcctggaaaaattacagaccttggatctccgagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttctcctacgggtacctcatctccagattctcattttaaatcaaaatcgcttctcctcctgtagtggagatcaaaccccttcagagaatcccagcttagaacagcttttccttggagaaaatatgttgcaacttgcctgggaaactgagctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagatactaaagaaagaaaaagaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaacaccatgttctgtctcactaacctccaaatggaaaataatagatctagaaaattgcaactgcc

SEQ ID NO: 5 - DNA, 4992 bp, Homo sapiens
gaagactccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggagccaccaacgtctgtcacaggagttggaaagatggggtttatataatgcatcaagtcttctttcttatctctctgtgtctctatttgcacttgagtctctcacctcagctcctgtaaaagagtggcaagtaaaaaacatggggctctgattctcctgtaattgtgataattaaatatacacacaatcatgacattgagaagaactgcatttctacccttaaaaagtactggtatatacagaaatagggttaaaaaaaactcaagctctctctatatgagaccaaaatgtactagagttagtttagtgaaataaaaaaccagtcagctggccgggcatggtggctcatgcttgtaatcccagcactttgggaggccgaggcaggtggatcacgaggtcaggagtttgagaccagtctggccaacatggtgaaaccccgtctgtactaaaaatacaaaaattagctgggcgtggtggtgggtgcctgtaatcccagctacttgggaggctgaggcaggagaatcgcttgaacccgggaggtggaggtggcagtgagccgagatcacgccactgcaatgcagcccgggcaacagagctagactgtctcaaaagaacaaaaaaaaaaaaacacaaaaaaactcagtcagcttcttaaccaattgcttccgtgtcatccagggccccattctgtgcagattgagtgtgggcaccacacaggtggttgctgcttcagtgcttcctgctctttttccttgggcctgcttctgggttccatagggaaacagtaagaaagaaagacacatccttaccataaatgcatatggtccacctacaaatagaaaaatatttaaatgatctgcctttatacaaagtgatattctctacctttgataatttacctgcttaaatgtttttatctgcactgcaaagtactgtatccaaagtaaaatttcctcatccaatatctttcaaactgttttgttaactaatgccatatatttgtaagtatctgcacacttgatacagcaacgttagatggttttgatggtaaaccctaaaggaggactccaagagtgtgtatttatttatagttttatcagagatgacaattatttgaatgccaattatatggattcctttcattttttgctggaggatgggagaagaaaccaaagtttatagaccttcacattgagaaagcttcagttttgaacttcagctatcagattcaaaaacaacagaaagaaccaagacattcttaagatgcctgtactttcagctgggtataaattcatgagttcaaagattgaaacctgaccaatttgctttatttcatggaagaagtgatctacaaaggtgtttgtgccatttggaaaacagcgtgcatgtgttcaagccttagattggcgatgtcgtattttcctcacgtgtggcaatgccaaaggctttactttacctgtgagtacacactatatgaattatttccaacgtacatttaatcaataagggtcacaaattcccaaatcaatctctggaataaatagagaggtaattaaattgctggagccaacta

SEQ ID NO: 6 - DNA, 4197 bp, Homo sapiens
ctcctgcatagagggtaccattctgcgctgctgcaagttacggaatgaaaaattagaacaacagaaacatggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggatccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaactgacgttaagtcatgatttcgcgccataataaagatgcaaaggaatgacatttctgtattagttatctattgctatgtaacaaattatcccaaaacttagtggtttaaaacaacacatttgctggcccacagtttttgagggtcaggagtccaggcccagcataactgggtcctctgctcagggtgtctcagaggctgcaatgtaggtgttcaccagagacataggcatcactggggtcacactcatgtggttgttttctggattcaattcctcctgggctattggccaaaggctatactcatgtaagccatgcgagcctctcccacaaggcagcttgcttcatcagagctagcaaaaaagagaggttgctagcaagatgaagtcacaatcttttgtaatcgaatcaaaaaagtgatatctcatcactttggccatattctatttgttagaagtaaaccacaggtcccaccagctccatgggagtgaccacctcagtccagggaaaacagctgaagaccaagatggtgagctctgattgcttcagttggtcatcaactattttcccttgactgctgtcctgggatggcctgctatcttgatgatagattgtgaatatcaggaggcagggatcactgtggaccatcttagcagttgacctaacacatcttcttttcaatatctaagaacttttgccactgtgactaatggtcctaatattaagctgttgtttatatttatcatatatctatggctacatggttatattatgctgtggttgcgttcggttttatttacagttgcttttacaaatatttgctgtaacatttgacttctaaggtttagatgccatttaagaactgagatggatagcttttaaagcatcttttacttcttaccattttttaaaagtatgcagctaaattcgaagcttttggtctatattgttaattgccattgctgtaaatcttaaaatgaatgaataaaaatgtttcattttacaa

SEQ ID NO: 7 - DNA, 3868 bp, Homo sapiens
ggaggtcttgtttccggaagatgttgcaaggctgtggtgaaggcaggtgcagcctagcctcctgctcaagctacaccctggccctccacgcatgaggccctgcagaactctggagatggtgcctacaagggcagaaaaggacaagtcggcagccgctgtcctgagggcaccagctgtggtgcaggagccaagacctgagggtggaagtgtcctcttagaatggggagtgcccagcaaggtgtacccgctactggtgctatccagaattcccatctctccctgctctctgcctgagctctgggccttagctcctccctgggcttggtagaggacaggtgtgaggccctcatgggatgtaggctgtctgagaggggagtggaaagaggaaggggtgaaggagctgtctgccatttgactatgcaaatggcctttgactcatgggaccctgtcctcctcactgggggcagggtggagtggagggggagctactaggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccacgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctatgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagcaggcactcaataaatgctaccgaaggc

SEQ ID NO: 8 - DNA, 3594 bp, Homo sapiens
gaatcatccacgcacctgcagctctgctgagagagtgcaagccgtgggaattcagcagctgaatatcaagacctttgaattcaacaagaagttaagacatttatagttgtctaacaacagactgaagattgtggcttggtattcactggcaggtttcagacatttagatctttcttttaatgactaacaccatgcctatctgtggagaagctggcaacatgtcacacctggaaattgtttttcaacattaatactattatttggcagtaatccagattgcttttgccaccaacctgaagacatatagaggcagaaggacaggaataattctatttgtttcctgttttgaaacttccatctgtaaggctatcaaaaggagatgtgagagagggtattgagtctggcctgacaatgcagttcttaaaccaaaggtccattatgcttctcctctctgagaatcctgacttacctcaacaacggagacatggcacagtagccagcttggagacttctcagccaatgctctgagatcaagtcgaagacccaatatacagggttttgagctcatcttcatcattcatatgaggaaataagtggtaaaatccttggaaatacaatgagactcatcagaaacatttacatattttgtagtattgttatgacagcagagggtgatgctccagagctgccagaagaaagggaactgatgaccaactgctccaacatgtctctaagaaaggttcccgcagacttgaccccagccacaacgacactggatttatcctataacctcctttttcaactccagagttcagattttcattctgtctccaaactgagagttttgattctatgccataacagaattcaacagctggatctcaaaacctttgaattcaacaaggagttaagatatttagatttgtctaataacagactgaagagtgtaacttggtatttactggcaggtctcaggtatttagatctttcttttaatgactttgacaccatgcctatctgtgaggaagctggcaacatgtcacacctggaaatcctaggtttgagtggggcaaaaatacaaaaatcagatttccagaaaattgctcatctgcatctaaatactgtcttcttaggattcagaactcttcctcattatgaagaaggtagcctgcccatcttaaacacaacaaaactgcacattgttttaccaatggacacaaatttctgggttcttttgcgtgatggaatcaagacttcaaaaatattagaaatgacaaatatagatggcaaaagccaatttgtaagttatgaaatgcaacgaaatcttagtttagaaaatgctaagacatcggttctattgcttaataaagttgatttactctgggacgaccttttccttatcttacaatttgtttggcatacatcagtggaacactttcagatccgaaatgtgacttttggtggtaaggcttatcttgaccacaattcatttgactactcaaatactgtaatgagaactataaaattggagcatgtacatttcagagtgttttacattcaacaggataaaatctatttgcttttgaccaaaatggacatagaaaacctgacaatatcaaatgcacaaatgccacacatgcttttcccgaattatcctacgaaattccaatatttaaattttgccaataatatcttaacagacgagttgtttaaaagaactatccaactgcctcacttgaaaactctcattttgaatggcaataaactggagacactttctttagtaagttgctttgctaacaacacacccttggaacacttggatctgagtcaaaatctattacaacataaaaatgatgaaaattgctcatggccagaaactgtggtcaatatgaatctgtcatacaataaattgtctgattctgtcttcaggtgcttgcccaaaagtattcaaatacttgacctaaataataaccaaatccaaactgtacctaaagagactattcatctgatggccttacgagaactaaatattgcatttaattttctaactgatctccctggatgcagtcatttcagtagactttcagttctgaacattgaaatgaacttcattctcagcccatctctggattttgttcagagctgccaggaagttaaaactctaaatgcgggaagaaatccattccggtgtacctgtgaattaaaaaatttcattcagcttgaaacatattcagaggtcatgatggttggatggtcagattcatacacctgtgaataccctttaaacctaaggggaactaggttaaaagacgttcatctccacgaattatcttgcaacacagctctgttgattgtcaccattgtggttattatgctagttctggggttggctgtggccttctgctgtctccactttgatctgccctggtatctcaggatgctaggtcaatgcacacaaacatggcacagggttaggaaaacaacccaagaacaactcaagagaaatgtccgattccacgcatttatttcatacagtgaacatgattctctgtgggtgaagaatgaattgatccccaatctagagaaggaagatggttctatcttgatttgcctttatgaaagctactttgaccctggcaaaagcattagtgaaaatattgtaagcttcattgagaaaagctataagtccatctttgttttgtctcccaactttgtccagaatgagtggtgccattatgaattctactttgcccaccacaatctcttccatgaaaattctgatcatataattcttatcttactggaacccattccattctattgcattcccaccaggtatcataaactgaaagctctcctggaaaaaaaagcatacttggaatggcccaaggataggcgtaaatgtgggcttttctgggcaaaccttcgagctgctattaatgttaatgtattagccaccagagaaatgtatgaactgcagacattcacagagttaaatgaagagtctcgaggttctacaatctctctgatgagaacagattgtctataaaatcccacagtccttgggaagttggggaccacatacactgttgggatgtacattgatacaacctttatgatggcaatttgacaatatttattaaaataaaaaatggttattcccttcatatcagtttctagaaggatttctaagaatgtatcctatagaaacaccttcacaagtttataagggcttatggaaaaaggtgttcatcccaggattgtttataatcatgaaaaatgtggccaggtgcagtggctcactcttgtaatcccagcactatgggaggccaaggtgggtgacccacgaggtcaagagatggagaccatcctggccaacatggtgaaaccctgtctctactaaaaatacaaaaattagctgggcgtgatggtgcacgcctgtagtcccagctacttgggaggctgaggcaggagaatcgcttgaacccgggaggtggcagttgcagtgagctgagatcgagccactgcactccagcctggtgacagagcgagactccatctcaaaaaaaagaaaaaaaaaaaagaaaaaaa

SEQ ID NO: 9 - DNA, 23 bp, Artificial Sequence
  Primer
atgggagaagtatgtgcatggtg

SEQ ID NO: 10 - DNA, 20 bp, Artificial Sequence
  Primer
gtcacgatgacgataccggc

SEQ ID NO: 11 - DNA, 17 bp, Artificial Sequence
  Primer
ttcgccttccacatgcg

SEQ ID NO: 12 - DNA, 16 bp, Artificial Sequence
  Primer
ccttcgtcggcccgtg

SEQ ID NO: 13 - DNA, 20 bp, Artificial Sequence
  Primer
agccagcatcgtacccctct

SEQ ID NO: 14 - DNA, 20 bp, Artificial Sequence
  Primer
ccggaacctggacagagtct

SEQ ID NO: 15 - DNA, 22 bp, Artificial Sequence
  Primer
tgcagatgaagctagaagcctg

SEQ ID NO: 16 - DNA, 19 bp, Artificial Sequence
  Primer
cgctcgaaggtcttgtacg

SEQ ID NO: 17 - DNA, 21 bp, Artificial Sequence
  Primer
atgcacacaaacatggcacag

SEQ ID NO: 18 - DNA, 20 bp, Artificial Sequence
  Primer
tacaggctaaggtgcgtaaa

SEQ ID NO: 19 - DNA, 19 bp, Artificial Sequence
  Primer
ccacgctgctgaacatgct

SEQ ID NO: 20 - DNA, 20 bp, Artificial Sequence
  Primer
cagtaggtcgtccacaagct

SEQ ID NO: 21 - DNA, 19 bp, Artificial Sequence
  Primer
ccacgctgctgaacatgct

SEQ ID NO: 22 - DNA, 20 bp, Artificial Sequence
  Primer
tcgaacacctgctggatgac

SEQ ID NO: 23 - DNA, 18 bp, Artificial Sequence
  Primer
cgtggtggacgcctttgt

SEQ ID NO: 24 - DNA, 20 bp, Artificial Sequence
  Primer
tccccataagccgtgaagtt

(19) United States
(12) Patent Application Publication
(10) Pub. No.: US 2009/0263492 A1
(43) Pub. Date: Oct. 22, 2009

(54) Diagnostic Methods and Genetic Markers for Alzheimer Disease

(76) Inventors: John R. Cashman, San Diego, CA (US); Milan Fiala, Los Angeles, CA (US)

Correspondence Address: JONES DAY, 222 EAST 41ST ST, NEW YORK, NY, 10017, US

(21) Appl. No.: 12/407,756

(22) Filed: Mar. 19, 2009

Related U.S. Application Data
(63) Continuation of application No. PCT/US2007/020411, filed on Sep. 19, 2007.
(60) Provisional application No. 60/845,539, filed on Sep. 19, 2006.
(60) Provisional application No. 60/881,800, filed on Jan. 23, 2007.
(60) Provisional application No. 60/931,854, filed on May 24, 2007.

(51) Int. Cl. A61K 35/14; A61K 31/12; C12Q 1/02; G01N 33/00; C07C 69/76; C07C 49/00; C12Q 1/48; A61P 25/28

(52) U.S. Cl. 424/529; 514/679; 435/29; 436/94; 560/55; 568/308; 435/15

(57) ABSTRACT

Disclosed are methods for identifying individuals suffering from a CNS disorder (including Alzheimer's Disease, behavioral disorders, and the like) that could be treated with a CNS drug with greater therapeutic efficacy and lower side effects and the compounds useful for such treatment. Also disclosed are methods for predicting the efficacy of a drug candidate for the treatment of a CNS disorder. The technology is also applicable to drug discovery for use in animal models of neurodegenerative diseases.

### CROSS-REFERENCES TO RELATED APPLICATIONS

This application is a continuation of the PCT/US2007/020411 filed Sep. 19, 2007, which claims priority to U.S. provisional application Ser. No. 60/845,539 filed Sep. 19, 2006, Ser. No. 60/881,800 filed Jan. 23, 2007, and Ser. No. 60/931,854 filed May 24, 2007, all of which are incorporated herein by references in their entireties.

### FIELD

Provided are genetic markers identified from isolated DNA molecules of individuals with clinically characterized Alzheimer's Disease (AD) consisting of genes and proteins that are associated with significantly elevated clinical efficacy of AD medications curcumin and curcumin analogs and related immune modulators. Also provided are compounds capable of up-regulation of N-acetylglucosaminyltransferase III (Mgat3) and Toll-like receptors (TLRs) and increase of phagocytosis of amyloid-β (1-42) (Aβ). Further provided is a diagnostic method for detecting down-regulated Mgat3 or TLRs or Mgat3 or TLR polymorphic variants and quantifying the potential for AD in biological samples.

### BACKGROUND

### Enhancement of the Innate Immune System

Treatment of Alzheimer's disease remains an elusive goal due to a poor understanding of its pathogenesis and due to the inability to diagnose the disease early in progression. Abeta (Aβ) accumulation in AD brain is related to abnormal cross-talk between Aβ reactive T cells and microglia leading to differentiation of microglia into either phagocytes or antigen presenting cells and inhibition of complement activation (Science 302 (2003) 834-838). It was shown that macrophages and microglia of middle-aged and older normal subjects perform Aβ clearance but this function is defective in AD patients (Journal of Alzheimer's Disease 7 (2005) 221-232) although no defect for AD patients has been detected in bacterial phagocytosis.

Chemical substances such as curcuminoids and the hormone insulin-like growth factor (IGF-I) can bolster the innate immune system and thus have epidemiologic and aging-related rationale for use in AD. The Aβ uptake by AD macrophages is significantly lower in comparison to control macrophages and involves surface binding but not intracellular phagocytosis. After treatment of AD macrophages with curcuminoids, Aβ uptake by macrophages of AD patients increases and induction of phagocytosis occurs. Therefore, enhancement of innate immunity might provide a natural non-toxic approach to AD therapy

Activated microglia is considered to be responsible for both brain inflammation and Aβ phagocytosis through various receptors. Immunohistochemical studies of AD brain showed that inducible nitric-oxide synthase-positive and cyclooxygenase-2-positive blood-borne monocytes/macrophages penetrate across brain microvessels and infiltrate perivascular and parenchymal sites but only partially clear neurotic plaques. This shows that in human AD brain, peripheral monocytes/macrophages are the cells involved in Aβ clearance. It was also shown that peripheral blood macrophages and T-cells are able to invade the brain of aged amyloid precursor protein transgenic (APP23) mice and clear Aβ deposits.

Currently, there is no clinically successful strategy to remove Aβ deposits from the brain. In sporadic cases of AD, amyloidosis of the brain may be related to defective clearance of Aβ which has led to development of an Aβ vaccine but its use in a clinical trial was abrogated due to adverse encephalitic complications.

Current transgenic animals do model brain amyloidosis, albeit iatrogenically, but they do not reproduce the immune problems of patients with AD. Therefore, studying the benefits of enhancing of immune response to Aβ using peripheral blood leukocytes of AD patients and control subjects has significant advantages. In cultured macrophages of AD patients in vitro, curcuminoids improve the defect in macrophage phagocytosis of Abeta of about 75% of the patients studied. This mechanism of action of curcuminoids is novel and not in line with anti-inflammatory and pro-apoptotic effects of curcuminoids. It is also shown that IGF-1 improves Aβ phagocytosis in macrophages of AD patients.

The effects of immunomodulating and anti-inflammatory therapies could be evaluated in vitro and individualized according to each subjects innate and adaptive immune responses. This requires information about genetic and biochemical markers of immune response that are described herein. As described below, curcuminoids upregulate the Mgat3 and TLR genes and this might be an important part of the neuroprotective mechanism of curcuminoids in AD.

Mgat 3 and TLRs in Neurodegeneration

Nearly all proteins of blood serum and on cell surfaces of higher organisms are glycosylated. The N-glycans of mammalian glycoproteins vary widely in structure, but contribute to important biological processes. N-Acetylglucosaminyltransferase III (Mgat 3), the product of the Mgat 3 gene, transfers the bisecting GlcNAc to the core mannose of complex N-glycans. Defective Mgat3 could markedly change cell-mediated immunity and the function of other N-glycosylated biomolecules. Individuals with defective or abnormal amount of Mgat3 may have other neurobiological problems. Individuals with mutations in the Mgat3 gene that lead to inactive Mgat3 might have neurological or behavioral problems similar to but milder than those observed for patients with certain congenital disorders of glycosylation. Loss of Mgat3 or decreased expression over time might also have deleterious consequences and Mgat3 loss might compromise the normal cell processes including cytoprotection in AD.

Toll-like receptors (TLRs) are a family of pattern-recognition receptors in the innate immune system. TLRs comprise a group of 10 genes and their gene products (i.e., TLR1-10). TLRs are cell-surface signaling receptors involved in host response. TLR agonists are being developed for the treatment of diseases that involve inappropriate adaptive immune diseases such as sepsis, autoimmune disease, cancer, allergies and viral and bacterial infections (Nat Med. 13, 552, 2007). TLR antagonists are being developed to combat inflammation and autoimmunity diseases. Most of the literature in this area has examined the role of inflammatory mediators in the activation of endogenous or exogenous microglia. For example, activation of microglia with a TLR ligand markedly boost ingestion of Aβ in vitro (Tahara et al., Brain 129, 3006, 2006). Activation of TLR2 markedly enhance mouse formyl peptide receptor-like 2 (mFPR2)-mediated uptake of Aβ by microglia (Chen et al., J. Biol. Chem. 281, 3651, 2006). Other studies have suggested that the TLR4 Asp299Gly variant may be protective toward the development of late-onset AD.

Curcuminoids enhance uptake of Aβ by macrophages of AD patients. Normal subjects' macrophages perform adequately without any treatment. Treatment with curcuminoids enhance not only the intensity of uptake but induce intracellular phagocytosis, reduce oxidative damage, interleukin-1 beta reactivity and microgliosis in a APPsw transgenic mouse model. Curcuminoids are also known to have anti-inflammatory properties and anti- and pro-apoptotic properties, which might modulate excessive inflammatory responses by macrophages. Other beneficial properties of curcuminoids, such as inhibition of Aβ aggregation, are also relevant to AD patients.

The enhancement of innate immune functions, phagocytosis and resistance to apoptosis by curcuminoids suggests that immune modulation of the innate immune system might be a safe alternative to vaccination. Therefore, the biochemical and functional defects of AD macrophages and their modulation by natural substances provide an entirely new direction to the pathogenesis of Alzheimer's disease and create new diagnostic and therapeutic opportunities in AD. Our results with peripheral monocytes and macrophages suggest that testing the status of innate immunity in AD patients would be helpful to assess the ability of patients to respond to immunomodulatory therapy with curcumins or related agents.

The human Mgat3 and Toll-like receptor (TLR) genes might be useful in testing other immune modulators or other drug candidates for CNS drug activity or neurodegenerative diseases including treatment and diagnosis of AD. The instant disclosure solves the problem of defects in phagocytosis of amyloid-β (1-42) (Aβ) of the innate immune cells, monocytes/macrophages of AD patients and in clearance of Aβ plaques by AD patients by identifying the active principle in crude curcuminoids and synthesizing more biologically active derivatives.

### SUMMARY

In one aspect, provided are Mgat3 and TLRs genes and corresponding proteins and/or variant forms of these proteins as biological markers (and/or drug targets) for modulation in vitro and/or in vivo as an indicator of CNS damage for a number of brain diseases or indicator of therapy. Mgat3 or TLR modulation represents a promising approach to protect individuals suffering from AD or other neurodegenerative diseases.

In another aspect, evaluation of Mgat3 and/or TLRs in isolated macrophages or modulation of Mgat3 or TLRs in vivo or ex vivo offers a clinically relevant diagnostic and therapeutic tool and provides an immediate approach to neurodegenerative disease diagnosis and treatment.

In yet another aspect, provided are therapeutic agents (curcumins and/or analogs thereof) that can be used to up-regulate Mgat3 and/or TLRs that facilitates Aβ plaque degradation and removal. The compounds having the following formula (I):

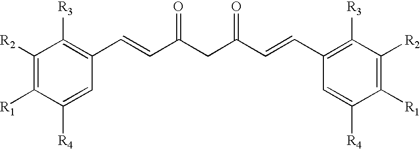

wherein R₁, R₂, R₃ and R₄ are as described below.

In another aspect, provided is a method for treating Alzheimer disease by administering to a patient in need of such treatment a curcumin or curcumin analog of the formula (I).

In another aspect, provided is a method for ex vivo stimulation of Mgat3 and/or TLRs comprising the steps of obtaining human blood cells, treating them with therapeutic agents and re-introducing the stimulated cells to stimulate Aβ plaque degradation and removal.

In another aspect, provided herein is a method to assess the profile of physiological, metabolic, genetic and biochemical signatures that can be derived and are predictive of the biological or physiological potential of a chemical or drug to promote human Aβ clearance. The instant disclosure solves the problem of predicting the potential of a chemical or drug as an anti-AD agent by identifying the effect on Aβ clearance at an early stage in an in vitro setting.

In another aspect, provided herein are novel agents capable of enhancing Aβ clearance.

In yet another aspect, provided are methods for in vitro screening of compounds for Aβ clearance potential or other biological activities by identifying biological parameters undergoing active change. These methods include incubating a chemical with a cell; determining the pathological, morphological and biochemical change and detection of the amount and type of cellular change produced.

In another aspect, provided are methods for in vitro screening of compounds for facilitating Aβ clearance potential or other biological activities of relevance to the in vivo condition.

In another aspect, provided is a method of predicting the potential of a chemical or drug as an anti-AD agent by identifying its effect on Aβ clearance at an early stage in an in vitro setting. In another embodiment, provided is a method of identifying individuals that harbor defective or low levels of Mgat3 or TLRs as biomarkers of use in predicting those individuals with AD or other neurodegenerative diseases.

In another aspect, provided is a method for predicting an efficacy of an AD drug in an individual, where said drug is a Mgat3 or TLR modulator and said individual is suffering from or at risk of developing a CNS disorder amenable to treatment with the drug, comprising the following steps: (1) isolating a biological sample from an individual, the biological sample comprising at least one of (i) nucleic acids and (ii) Mgat3 proteins (or general N-glycosylated proteins) or TLR; and (2) analyzing the biological sample to determine in the individual the presence or absence of Mgat3 or TLR alleles or the Mgat or TLR gene, where the relative amount of the Mgat3 or TLR gene is indicative of a positive clinical outcome for treatment of the disorder with the drug. In certain embodiments, the CNS disorder is a neurodegenerative disorder (e.g., AD). The methods are particularly suitable for use where, for example, drug has a relationship to anti-AD (e.g., the agent is a curcuminoid or analog). In one embodiment, the biological sample comprises nucleic acids. In another embodiment, the analyzing step comprises analyzing the nucleic acids from the biological sample to determine the nucleotides present in the Mgat3 or TLR gene coding region. In yet another embodiment, the method can further include determining the Mgat3 or TLR genotype at other nucleotide positions of the Mgat3 or TLR gene coding region, non-coding region or promoter region. In another embodiment, the analyzing step comprises hybridization between said nucleic acid sample and a nucleic acid selected from the group consisting of (a) a nucleic acid comprising at least 10 to 100 contiguous nucleotides of the nucleotide sequences set forth in SEQ ID NO: 1, where the nucleic acid includes the nucleotide at key Mgat3 or TLR alleles and/or a base adjacent thereto; and (b) a nucleic acid that is fully complementary to the nucleic acid of (a). In certain embodiments, the nucleic acid is conjugated with a detectable marker or agent to assist in isolation.

In another aspect, provided is a method for predicting the efficacy of a candidate agent for the treatment of a CNS disorder, where the candidate agent is a derivatized or modified form of a predetermined therapeutic agent for the treatment of the disorder, comprising the following steps: (1) contacting a first AD sample of an Mgat3 or TLR protein with the candidate agent; (2) contacting a second normal sample of an Mgat3 or TLR protein with the predetermined therapeutic agent; where the contacting of each of the first and second samples is under conditions suitable for affording Mgat3 enzyme or TLR activity; (3) determining for each of the first and second samples the level of Mgat3 enzyme or TLR activity; and (4) comparing the level of Mgat3 enzyme or TLR activity in the first sample with the level of Mgat3 enzyme or TLR activity in the second sample, whereby a greater level of Mgat3 enzyme or TLR activity in the first sample relative to the second sample is indicative of efficacy of the candidate agent. In certain embodiments, a control used is the cDNA-expressed form of Mgat3 or TLR. In certain embodiments, the CNS disorder is a neurodegenerative disorder. In certain embodiments, the predetermined therapeutic agent is a curcuminoid or derivative or some other immune-modulating agent. In certain embodiments, drug candidates are agents that have been derivatized to incorporate an Mgat3 substrate moiety (e.g., a curcuminoid-like center).

In some embodiments, the method for determining the level of Mgat3 enzyme or TLR activity comprises detecting the level of an N-glycosylated metabolite of the cell in a sample.

In another embodiment, provided is a method for ex vivo immunotherapy for patients with AD.

### BRIEF DESCRIPTION OF THE DRAWINGS

FIGS. 1A to 1J demonstrate transcription of Toll-like receptor (TLR) RNA's in PBMC's cells and up-regulation of TLR's by bisdemethoxycurcumin. 10 million PBMC's each of four AD patients (A, B, C, D) were treated overnight with no addition, Aβ (A, B, C, D; black bars) or with Aβ and bisdemethoxycurcumin (D, striped bar). Control subjects's (M, N, O) PBMC's were treated with no addition or Aβ (open bars). RNA was extracted and tested by qPCR, and the TLR ratio was determined as described in the Methods. The significance of differences between patients and controls (by Mann-Whitney test) are: TLR1 0.05; TLR2 0.05; TLR3 0.05; TLR4 0.08; TLR5 0.05; TLR6 0.077; TLR7 0.08; TLR8 0.05; TLR9 0.08; TLR10 0.05. The graphs show changes in receptors on 2-fold (log 2) scale.

### DETAILED DESCRIPTION

### Definitions

Unless defined otherwise, all technical and scientific terms used herein have the same meaning as commonly understood by one of ordinary skill in the art to which this subject matter pertains. Although any methods and material similar to those described herein can be used in the practice or testing of the present disclosure, only exemplary methods and materials are described.

The following terms are defined below where R refers to the R in Schemes 1 or 2.

The terms “a,” “an,” and “the” include plural referents unless the context clearly dictates otherwise.

The term “hydrido” refers to a single hydrogen.

The term “alkyl” refers to saturated aliphatic groups including straight chain, branched chain, and cyclic groups, all of which may be optionally substituted. Suitable alkyl groups include methyl, ethyl and the like, and may be optionally substituted.

The term “alkenyl” refers to unsaturated groups which contain at least one carbon-carbon double bond and includes straight chain, branched chain, and cyclic groups, all of which may be optionally substituted.

The term “alkynyl” refers to unsaturated groups which contain at least one carbon-carbon triple bond and includes straight chain, branched chain, and cyclic groups, all of which may be optionally substituted. Suitable alkynyl groups include ethynyl, propynyl, butynyl and the like which may be optionally substituted.

The term “alkoxy” refers to the ether —OR where R is alkyl, alkenyl, alkynyl, aryl, or aralkyl.

The term “aryloxy” refers to the ether —OR where R is aryl or heteroaryl.

The term “alkenyloxy” refers to ether —OR where R is alkenyl.

The term “alkylthio” refers to —SR where R is alkyl, alkenyl, alkynyl, aryl, aralkyl.

The term “alkylthioalkyl” refers to an alkylthio group attached to an alkyl radical of about one to twenty carbon atoms through a divalent sulfur atom.

The term “alkylsulfinyl” refers to —S(O)R where R is alkyl, alkenyl, alkynyl, aryl, aralkyl.

The term “sulfonyl” refers to a —SO₂—R group where R is alkyl, alkenyl, alkynyl, aryl, or aralkyl.

The term “aminosulfonyl”, “sulfamyl”, “sulfonamidyl” refer to —SO₂NRR′ where R and R′ are independently selected from alkyl, alkenyl, alkynyl, aryl, and aralkyl.

The term “hydroxyalkyl” refers to linear or branched alkyl radicals having one to about twenty carbon atoms any one of which may be substituted with a hydroxyl group.

The term “cyanoalkyl” refers to linear or branched alkyl radicals having one to about twenty carbon atoms any one of which could be substituted with one or more cyano groups.

The term “alkoxyalkyl” refers to alkyl groups having one or more alkoxy radicals attached to the alkyl group. The alkoxy radical may be further substituted with one or more halo atoms. Preferred haloalkoxy groups may contain one to twenty carbons.

The term “oximinoalkoxy” refers to alkoxy radicals having one to about twenty carbon atoms, any one of which may be substituted with an oximino radical.

The term “aryl” refers to aromatic groups which have at least one ring having conjugated “pi” electron system and includes carbocyclic aryl, biaryl, both of which may be optionally substituted.

The term “carbocyclic aryl” refers to groups wherein the ring atoms on the aromatic ring are carbon atoms. Carbocyclic groups include phenyl and naphthyl groups which may be optionally substituted with 1 to 5 substituents such as alkyl, alkoxy, amino, amido, cyano, carboxylate ester, hydroxyl, halogen, acyl, nitro.

The term “aralkyl” refers to an alkyl group substituted with an aryl group. Suitable aralkyl groups include benzyl, and the like, and may be optionally substituted.

The term “aroyl” refers to —C(O)R where R is aryl group.

The term “alkoxycarbonyl” refers to —C(O)OR wherein R is alkyl, akenyl, alkynyl, aryl, or aralkyl.

The term “acyl” refers to the alkanoyl group C(O)R where R is, alkenyl, alkynyl, aryl, or aralkyl.

The term “acyloxy” refers to the alkanoyl group —OC(O)R where R is, alkenyl, alkynyl, aryl, or aralkyl.

The term “aminoalkyl” refers to alkyl which is substituted with amino groups.

The term “arylamino” refers to amino groups substituted with one or more aryl radicals.

The term “aminocarbonyl” refers to —C(O)NRR₁ wherein R and R₁ are independently selected from hydrogen, alkyl, akenyl, alkynyl, aryl, and aralkyl.

The azidoalkyl refers to alkyl R which is substituted with azido —N₃.

The term “amino” refers to —NRR₁ where R and R₁ are independently hydrogen, lower alkyl or are joined together to give a 5 or 6-membered ring such as pyrrolidine or piperidine rings which are optionally substituted.

The term “alkylamino” includes amino groups substituted with one or more alkyl groups.

The term “dialkylamino” refers to —NRR₁ R and R₁ are independently lower alkyl groups or together form the rest of ring such as morpholino. Suitable dialkylamino groups include dimethylamino, diethylamino and morpholino.

The term “morpholinoalkyl” refers to alkyl R substituted with morpholine group.

The term “isocyanoalkyl” refers to alkyl R that is substituted with isocyano group —NCO.

The term “isothiocyanoalkyl” refers to alkyl R that is substituted with isothiocyano group —NCS.

The term “isocyanoalkenyl” refers to alkenyl R that is substituted with isocyano group —NCO.

The term “isothiocyanoalkenyl” refers to alkenyl R that is substituted with isothiocyano group —NCS.

The term “isocyanoalkynyl” refers to alkynyl R that is substituted with isocyano group —NCO.

The term “isothiocyanoalkynyl” refers to alkynyl R that is substituted with isothiocyano group —NCS.

The term “alkanoylamino” refers to —NRC(O)OR₁ where R and R₁ are independently hydrogen, lower alkyl, akenyl, alkynyl, aryl, or aralkyl.

The term “formylalkyl” refers to alkyl R substituted with CHO.

The term “optionally substituted” or “substituted” refers to groups substituted by one to five substituents, indepentyl selected from lower alkyl (acyclic or cyclic), aryl (carboaryl or heteroaryl) alkenyl, alkynyl, alkoxy, halo, haloalkyl (including trihaloalkyl, such as trifluoromeyl), amino, mercapto, alkylthio, alkylsulfinyl, alkylsulfonyl, nitro, alkanoyl, alkanoyloxy, alkanoyloxyalkanoyl, alkoxycarboxy, (—COOR, where R is lower alkyl), aminocarbonyl (—CONRR₁, where R and R₁ are indepentyl lower alkyl), formyl, carboxyl, hydroxyl, cyano, azido, keto, and cyclic ketals thereof, alkanoylamido, heteroaryloxy, and heterocarbocyclicoxy.

The term “lower” refers herein in connection with organic radicals or compounds defines such as one up to and including ten, preferably up to and including six, and more preferably one to four carbon atoms. Such groups may be straight chain, branched chain, or cyclic.

The term “heterocyclic” refers to carbon containing radicals having three, four, five, six, or seven membered rings and one, two, three, or four O, N, P, or S heteroatoms, e.g., thiazolidine, tetrahydrofuran, 1,4-dioxane, 1,3,5-trioxane, pyrrolidine, pyridyl, piperidine, quinuclidine, dithiane, tetrahydropyran, and morpholine or fused analogs containing any of the above.

The term “heteroaryl” refers to carbon containing 5-14 membered cyclic unsaturated radicals containing one, two, three, or four O, N, P, or S atoms and having 6, 10 or 14π electrons delocalized in one or more than one rings, e.g., pyridine, oxazole, indole, purine, pyrimidine, imidazole, benzimidazole, indazole, 2H-1,2-4-triazole, 1,2,3-triazole, 2H-1,2,3,4-tetrazole, 1H-1,2,3,4-triazolebenztriazole, 1,2,3-triazolo[4,5-b]pyridine, thiazole, isoxazole, pyrazole, quinoline, cytosine, thymine, uracil, adenine, guanine, pyrazine, picoline, picolinic acid, furoic acid, furfural, furyl alcohol, carbazole, isoquinoline, pyrrole, thiophene, furan, phenoxazine, and phenothiazine, each of which may be optionally substituted.

The term “pharmaceutically acceptable esters, amides, or salts” refers to esters, amides, or salts of compounds of Scheme 1 derived from the combination of a compound and an organic or inorganic acid provided herein.

The term “curcumin-related agent” refers to curcumin-related compounds, curcumin metabolites, curcumin analogues, and curcumin derivatives, as further described herein.

The term “inhibit” means to reduce by a measurable amount, or to prevent entirely.

“Treating,” “treatment,” or “therapy” of a disease or disorder means slowing, stopping, or reversing progression of the disease or disorder, as evidenced by a reduction or elimination of either clinical or diagnostic symptoms, using the compositions and methods as described herein.

“Preventing,” “prophylaxis,” or “prevention” of a disease or disorder means prevention of the occurrence or onset of a disease or disorder or some or all of its symptoms.

The term “subject” as used herein means any mammalian patient to which the compositions provided herein may be administered according to the methods described herein. Subjects specifically intended for treatment or prophylaxis using the methods provided herein include humans.

The term “therapeutically effective regime” means that a pharmaceutical composition or combination thereof is administered in sufficient amount and frequency and by an appropriate route to at least detectably prevent, delay, inhibit, or reverse development of at least one symptom or biochemical marker of a neurodegenerative-related disorder. In certain embodiments, the “therapeutically effective regime” predisposes a subject to improve cognition, memory and other aspects of AD.

The term “therapeutically effective amount” refers to an amount of an anti-AD-related agent, or a combination of a anti-AD-related agent with other agent(s), to achieve a desired result, e.g., preventing, delaying, inhibiting, or reversing a symptom or biochemical marker of a neurodegenerative disorder or AD when administered in an appropriate regime.

“Amenable to treatment” with the drug means that the disorder is either predicted or determined to be a disorder that can be treated by administration of the drug (for example, through clinical testing such as by, e.g., clinical trials conducted to obtain governmental approval of a drug).

The term “positive clinical outcome” refers to any improvement, or decrease in frequency of, clinical symptoms associated with the disorder, as determined using known diagnostic methods. Generally, indication of a positive clinical outcome using the above method is indicative of greater efficacy of the drug in the individual relative to an individual in which the Mgat3 or TLRs are absent.

Mgat3 or TLR inducers or up-regulation moieties as used herein refer to any chemical moiety that is known or predicted to up-regulate, modulate or induce by interaction with the Mgat3 or TLRs protein or gene during interaction of Mgat3 or TLRs with an agent having the chemical moiety.

Compounds

In one embodiment, provided are compounds having the following formula (I):

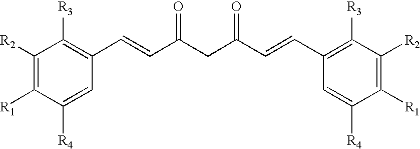

wherein R₁, R₂, R₃ and R₄ are independently selected from the group consisting of hydrogen, (C1-C6)alkyl, (C1-C6)alkenyl, (C1-C6)alkynyl, heteroalkyl, halo (e.g., fluoro, chloro, bromo, iodo), (C1-C6)alkoxy, amino, (C1-C6)alkylamino, hydroxy, cyano, nitro, 5- or 6-member optionally substituted unsaturated, partially unsaturated or saturated heterocyclyl or carbocyclyl optionally substituted with acyl, halo, lower acyl, lower haloalkyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, alkyl sulfinyl and aryl, lower aralkylthio, lower alkylsulfinyl, lower alkylsulfonyl, aminosulfonyl, lower N-arylaminosulfonyl, lower arylsulfonyl, lower N-alkyl-N-arylaminosulfonyl; aryl selected from the group consisting of phenyl, biphenyl, naphthyl, and 5- and 6-membered heteroaryl optionally substituted with one, two, or three substituents selected from halo, hydroxyl, amino, nitro, cyano, carbamoyl, lower alkyl, lower alkenyloxy, lower alkoxy, lower alkylthio, lower alkylsulfinyl, lower alkylsulfonyl, lower alkylamino, lower dialkylamino, lower haloalkyl, lower alkoxycarbonyl, lower N-alkylcarbamoyl, lower N,N-dialkylcarbamoyl, lower alkanoylamino, lower cyanoalkoxy, lower carbamoylalkoxy, and lower carbonylalkoxy; and wherein further the acyl group is optionally substituted with a substituent selected from hydrido, alkyl, halo, and alkoxy.

In certain embodiments, R₁, R₂, R₃, and R₄ is independently aryl having one or two ring hydrogens substituted with substituents selected from Cl, Br, I, —OR₄, —R₅, —OC(O)R₆, OC(O)NR₇R₈, —C(O)R₉, —CN, —NR₁₀R₁₁, —SR₁₂, —S(O)R₁₁, —S(O)₂R₁₄, —C(O)OR₁₅, —S(O)₂NR₁₆R₁₇; —R₁₈NR₁₉R₂₀ wherein R₄, R₅, R₆, R₇, R₈, R₉, R₁₀, R₁₁, R₁₂, R₁₃, R₁₄, R₁₅, R₁₆, R₁₇, R₁₈, R₁₉, and R₂₀ are the same or different and are branched or unbranched alkyl groups from one to eight carbon atoms or hydrogen radicals.

In another embodiment, R₁, R₂, R₃, and R₄ are each hydrogen.

In yet another embodiment, R₁, R₂, R₃, and R₄ are each a 5-membered heterocyclic or carbocyclic ring. In certain embodiments, R₁, R₂, R₃, and R₄ are each optionally substituted 5-membered ring having one or two heteroatoms selected from O, N and S. In specific embodiments, the heteroatom is selected from O or S.

In yet still further embodiments, R₁, R₂, R₃, and R₄ are each a 6-membered heterocyclic or carbocyclic ring. In certain embodiments, R₁, R₂, R₃, and R₄ are each optionally substituted 6-membered ring having one heteroatom selected from O, N and S. In certain embodiments, R₁, R₂, R₃, and R₄ are each optionally substituted 6-membered ring having two heteroatoms selected from O, N, and S.

In another embodiment, the compounds are selected from the compounds shown in the examples.

Methods of Use

The methods described herein are based in part on the applicants' discovery that the presence of the human Mgat3 and/or TLR gene and the corresponding gene product enzyme activity is predictive of the efficacy of CNS (e.g., anti-AD) drugs. The detection of polymorphisms in the Mgat3 or TLR genes are useful for designing prophylactic and/or therapeutic regimes customized to underlying abnormalities associated with CNS disease such as, for example, neurodegenerative disorders (e.g., AD, behavioral disorders, and the like). These methods are also useful for the pre-clinical development of drugs for treating CNS disorders, as well as for conducting clinical trials of drugs for treatment of these diseases and the underlying biological abnormalities.

We suggest that the key problem in AD lies specifically in the dysfunction of macrophages. Our studies of over 100 AD patients and approximately 40 control subjects reveal unsuspected pathophysiology of AD monocytes/macrophages, which is not explained by serum factors because they are observed even in the presence of fetal bovine serum. Heterogeneous defects in macrophage differentiation in vitro, abnormal Aβ uptake and trafficking to lysosomes, and apoptosis on exposure to Aβ has been observed. In addition, patients' monocytes over-express IL-12 and patients' CD4 T cells over produce IL-10 and interferon-gamma, the cytokines belonging to both T_{H}1 and T_{H}2 sets. Thus, the adaptive and innate immune system components of AD patients appear to be in various stages of disharmony and dysfunction. In contrast, macrophages of age-matched control subjects voraciously ingest Aβ and seem to degrade it. We believe that the whole innate immune system (including macrophages and microglia) in AD patients may be defective and its pathological state can be evaluated by studying peripheral blood monocytes/macrophages, genetic markers and enzyme activities.

In one embodiment, provided are methods for treatment of AD comprising administering to a subject in need of such treatment a curcumin or curcumin analog having formula (I).

In another embodiment, provided are methods for identifying individuals susceptible to suffering from AD, behavioral disorders, or other CNS diseases that could be more effectively treated with immune modulators (or other anti-AD drugs) with greater therapeutic efficacy and lower side effects. The present methods are particularly useful for determining such therapeutic efficacy and/or reducing toxicity, in individuals suffering from a wide number of CNS diseases, quickly and efficiently.

It may be that certain variants of Mgat3 or TLRs are markers for more efficacious AD therapy. Testing new drugs in populations of individuals suffering from AD, behavioral disorders or other CNS conditions that encoded variants of human Mgat3 or TLRs could provide substantial improvement in therapeutic efficacy and drug discovery. The Mgat3 or TLRs present in recombinant preparations are also useful in in vitro methods to identify drug candidates that are up-regulators for Mgat3 or TLRs that possess superior pharmacological or pharmaceutical properties useful in drug discovery and AD drug development. Thus, screening for Mgat3 or TLR inducers or modulators provides important information as to how to modify the drug candidate to make a drug having a greater therapeutic index and/or decreased toxicity. Human Mgat3 or TLR variants are also useful as a chemical or drug discovery agent in its own right as a means of identifying more highly efficacious drugs.

Further provided are methods of use the amino acid differences of human Mgat3 or TLRs to identify new human Mgat3 or TLRs up-regulators that may have superior drug development potential and find use as a bioindicator for drug development in the biotechnology or pharmaceutical industry.

In one embodiment, provided is a method for predicting in an individual the efficacy of a drug, where the drug is an Mgat3 or TLRs up-regulator or modulator and the individual is suffering from or at risk of developing a CNS disorder amenable to treatment with the drug. The method generally comprises (1) isolating a biological sample from an individual, where the biological sample includes nucleic acids and/or cellular proteins, and (2) analyzing the biological sample to determine in the individual the presence or absence of the Mgat3 or TLR gene and/or protein. A determination of the presence of the Mgat3 or TLR gene level or enzyme activity is indicative of a positive clinical outcome with administration of the drug for treating the CNS disorder.

In certain embodiments, where the biological sample includes cellular proteins from a tissue that expresses the Mgat3 or TLR genes, the Mgat3 or TLR protein in the sample is analyzed for the presence of the Mgat3 or TLR activity. For example, the determination of the presence in a sample of Mgat3 or TLRs can be carried out as an immunoassay in which the sample is contacted with antibodies capable of binding the Mgat3 or TLR protein. Antibodies (e.g., monoclonal antibodies) can be raised that specifically distinguish between wild-type Mgat3 or TLRs and any Mgat3 or TLRs variant. Methods for making antibodies are well-known in the art and are described in, e.g., Harlow and Lane, Antibodies: A Laboratory Manual (Cold Spring Harbor Laboratory, Cold Spring Harbor, N.Y. 1988).

In certain embodiments, the biological sample includes nucleic acids and the sample is analyzed to determine the nucleotide present at positions of codons of the Mgat3 or TLR genes (corresponding to nucleotide positions of SEQ ID NOs: 1-8 shown below).

| DNA Sequence of Human Mgat3, NM 002409 | | |
|---|---|---|
| 1 | gagcggccgc gccgggtccc cgggacgggg tggaagtggg ggtgggggga ggggatcggg | (SEQ ID NO: 1) |
| 61 | gccgggccgg ggccgcgctg cctgcgatgc cgggcgcccg ccgcagccgc tgccgccgga | |
| 121 | gcccgggatg gggcgagagg ctgcggcgga cgccagcatc tccccgccgg ggaccccggg | |
| 181 | ggccgcggag ccgccgccgc cgctgctgcc gccgttgctg agacccagcg ggcgatggga | |
| 241 | tgaagatgag acgctacaag ctctttctca tgttctgtat ggccggcctg tgcctcatct | |
| 301 | ccttcctgca cttcttcaag accctgtcct atgtcacctt cccccgagaa ctggcctccc | |
| 361 | tcagccctaa cctggtgtcc agctttttct ggaacaatgc cccggtcacg ccccaggcca | |
| 421 | gccccgagcc aggaggccct gacctgctgc gtaccccact ctactcccac tcgcccctgc | |
| 481 | tgcagccgct gccgcccagc aaggcggccg aggagctcca ccgggtggac ttggtgctgc | |
| 541 | ccgaggacac caccgagtat ttcgtgcgca ccaaggccgg cggcgtctgc ttcaaacccg | |
| 601 | gcaccaagat gctggagagg ccgcccccgg gacggccgga ggagaagcct gagggggcca | |
| 661 | acggctcctc ggcccggcgg ccaccccggt acctcctgag cgcccgggag cgcacggggg | |
| 721 | gccgaggcgc ccggcgcaag tgggtggagt gcgtgtgcct gcccggctgg cacggaccca | |
| 781 | gctgcggcgt gcccactgtg gtgcagtact ccaacctgcc caccaaggag cggctggtgc | |
| 841 | ccagggaggt gccgcgccgc gtcatcaacg ccatcaacgt caaccacgag ttcgacctgc | |
| 901 | tggacgtgcg cttccacgag ctgggcgacg tggtggacgc ctttgtggtg tgcgagtcca | |
| 961 | acttcacggc ttatggggag ccgcggccgc tcaagttccg ggagatgctg accaatggca | |
| 1021 | ccttcgagta catccgccac aaggtgctct atgtcttcct ggaccacttc ccgcccggcg | |
| 1081 | gccggcagga cggctggatc gccgacgact acctgcgcac cttcctcacc caggacggcg | |
| 1141 | tctcgcggct gcgcaacctg cggcccgacg acgtcttcat cattgacgat gcggacgaga | |
| 1201 | tcccggcccg tgacggcgtc cttttcctca agctctacga tggctggacc gagcccttcg | |
| 1261 | ccttccacat gcgcaagtcg ctctacggct tcttctggaa gcagccgggc accctggagg | |
| 1321 | tggtgtcagg ctgcacggtg gacatgctgc aggcagtgta tgggctggac ggcatccgcc | |
| 1381 | tgcgccgccg ccagtactac accatgccca acttcagaca gtatgagaac cgcaccggcc | |
| 1441 | acatcctggt gcagtggtcg ctgggcagcc ccctgcactt cgccggctgg cactgctcct | |
| 1501 | ggtgcttcac gcccgagggc atctacttca agctcgtgtc cgcccagaat ggcgacttcc | |
| 1561 | cacgctgggg tgactacgag gacaagcggg acctgaacta catccgcggc ctgatccgca | |
| 1621 | ccgggggctg gttcgacggc acgcagcagg agtacccgcc tgcagacccc agcgagcaca | |
| 1681 | tgtatgcgcc caagtacctg ctgaagaact acgaccggtt ccactacctg ctggacaacc | |
| 1741 | cctaccagga gcccaggagc acggcggcgg gcgggtggcg ccacaggggt cccgagggaa | |
| 1801 | ggccgcccgc ccggggcaaa ctggacgagg cggaagtcta gagctgcatg atctgatagg | |
| 1861 | gtttgtgaca gggcgggggt ggcggcggcc cctagcgcta tctccctgcc tcctgccggc | |
| 1921 | tccttggttc ttgaggggac caggagtggg tggggagtgg gggtgggggt agggtttccc | |
| 1981 | tactgaagcc cttgtgaatc aagggtcagg cctttgagct cagaaaatat ccctcctgtt | |
| 2041 | gggagagggc gcaggccgtg acgtctgggt ggcccttatg actgccaaga ctgctgtggc | |
| 2101 | caggaggtgc cactggagtg tgcgtggtgg tccctgggta gcgggggagg gtaggcagga | |
| 2161 | ttggggaaga gagcctgcag gatctcacca ggcagcctct ggggggtggc caggccggga | |
| 2221 | aaaagcccac catttggcat ccctgggcct tgggctccgt gtgggagacc ggcctgccag | |
| 2281 | gaggacccag ggctctgtaa gtagatgcat ttgggtccag gaggaagcgt ggacacctcg | |
| 2341 | tagggaagag atgaaaaagc cacatcctac caagaggagg tgctgaggga tgctttgcag | |
| 2401 | tgtagtcaga agtgctgggc cagatggaga cagaactcca ccccctgccg caaaggacag | |
| 2461 | gacctggctg ccctgggatg ctggtgcctg agtctgtctc tgtgcacccc tcaggctgtc | |
| 2521 | gtgagccaac acaggggcct ggagaaccct gaggagcttt ccttttggtt ctaaacccgg | |
| 2581 | cgttgacgtt ccttctccct ttcacattgc tgtcttgtgg actgtgcact cagtccttgc | |
| 2641 | aaggccaaga gtccagttgt aggtgtggcc ttgaggggga agtggggagg agaagactga | |
| 2701 | catgagtcct ctgcacggat ccgtctctcc ctccccatca ccccttcctt ctgacaccca | |
| 2761 | gtcccagctg tccactgtcc caggtgcagt cactgttgtg cccttccttg gggcaggctg | |
| 2821 | gctgggggcc agaaaggggc catgaggctg tcttgggccc aaaaagggac aataaggcca | |
| 2881 | gttgtatgct tcctgttcct catagcttgc cttggtgggg atgtctttgt tggagttgat | |
| 2941 | tctgagctgc tgtgattagg agaccctgaa atacagtggt ttaagcaaga tggaagcttg | |
| 3001 | tttctaatta gtctagattg agatggccca gagctggtag ggcagctctg cgtttcttca | |
| 3061 | tacgcacctt ccaattctgg gtacacagcg gctgctccag cgcccaccct cctgtgtgca | |
| 3121 | tccaagcctg ggggaagcag aaatagacaa gagggcacac ccactttttg ctaaaggcat | |
| 3181 | gagccagaat tggcaggctc acctctgctg gcctctcatt ggctgggact cagtcacatg | |
| 3241 | gccacaagca gctgctaggg aacctgggaa gtgtagtctt cagcggggcc gccatgtgcc | |
| 3301 | tggcctcacc ttgggagtta tcttattgat ggaggagaag agaatggata tgggggacca | |
| 3361 | gtagcatctc tgggagaggg ggagggagca gcaataactc agtcgtcgga tccagctctc | |
| 3421 | attgtcagag tttccggaac agcttgctcc tgtttccctc actgtgcagc ccagggctgg | |
| 3481 | gggcagtgag gagcttgcag ctctgtggga aggggaaaca ccccctcccc tcggcccctc | |
| 3541 | agacgctacc caatgatgcc ggtttgcaga gttggcctgt ggaatggctc atgtttgtgc | |
| 3601 | gtgtgtgtgt gtatatttat gggcatgggt gcatgcttgg tgtgtatttg tacatgtctg | |
| 3661 | tattgctgtg tccctgtaaa tacatgcttg tgtatggatg gaagaggcca ggcccaggcc | |
| 3721 | tggcctcttc ctcgggcctg tggccacacc tcctgcagct ccccaaaatg actgaggcag | |
| 3781 | aaagcccttg gggagcctag aaagcaaagc taaaggggat gcagggtctg tctgtctgtc | |
| 3841 | tgtctttcag tctgaggaat gagaatcctg acctgagggc tgtgcagctg agagcccact | |
| 3901 | acctccccag cccctctcgg ccccagccgc atcatcccac ctgtcccctc ccccccacct | |
| 3961 | ccagtggggc tttctccaga tgtcttatgg ttgggggttt cctgatgggc caggagagga | |
| 4021 | gggcatcttc ttgcgacagc actgtctggg ttaagtgccc agtgagggca tggtgtgggg | |
| 4081 | agctggcctc agaggagccg ctggtgggca agcgtgaagt gggctgaggg gctctgagcc | |
| 4141 | actttgctcc catctagggg actgcccccc atggaactcc tttgaagtca cagcagcctt | |
| 4201 | cctttctgtt tgctcttggg gctgagaggt ggctcaaaca ctcggggtcc ctatggctct | |
| 4261 | gggtcaatct aggccaggct gcaccccatg gacagggagt ctcagggctc ctgatcatgc | |
| 4321 | ccaggccctg gcctggggcc tccctccttg gcagctttcc cacccccacg cccctggcat | |
| 4381 | cctcagttgc tatgggatgc ccctccaggg caccagctca gggctaagcg aaggaagata | |
| 4441 | ggagcagctc agagctgcca ggctctgcct tcctcacaga cctggtgggg caggtcctgt | |
| 4501 | tcacagcagc aggagtgaag gcctggccat cggtggagag ggcagctgtc agagggctgg | |
| 4561 | gggccagggc acaggattga agagtttcac atatcatcac agcatacact gggaatttgg | |
| 4621 | tgggggcaga agaacccagg gccactccct caatatgaag ggaaaccaag ctgaatgtga | |
| 4681 | ccaccggcac actgctgcca tgtcccatgt ccacctttct ccccgggaat aactggccct | |
| 4741 | gagaccccta gacccaagga ggcctgtcca tgccaagcat ccgggaagca tggctggcct | |
| 4801 | tatccaccca tgggtcacgt cggttcccag gggcagcatg ggagatcttt gggggcaaca | |
| 4861 | gggagagtct gggtggggag acgggacttg tccaagcaga aggcaggacc ctgggaaatg | |
| 4921 | cataatgtaa ggacatcaat aatagtatta ttttttttgt aagggaaaat caatatgtac | |
| 4981 | attctgaaat cattttctct gtaaatggtt ggatttcatt tcacccttaa agggatgctt | |
| 5041 | aaaggagaag ataatattaa taataaaaac agctacaaag tctgaaaaaa aaaaaaaaaa | |
| 5101 | aa | |
| DNA Sequence of TLR 3, NM 0032675 | | |
| 1 | cactttcgag agtgccgtct atttgccaca cacttccctg atgaaatgtc tggatttgga | (SEQ ID NO: 2) |
| 61 | ctaaagaaaa aaggaaaggc tagcagtcat ccaacagaat catgagacag actttgcctt | |
| 121 | gtatctactt ttgggggggc cttttgccct ttgggatgct gtgtgcatcc tccaccacca | |
| 181 | agtgcactgt tagccatgaa gttgctgact gcagccacct gaagttgact caggtacccg | |
| 241 | atgatctacc cacaaacata acagtgttga accttaccca taatcaactc agaagattac | |
| 301 | cagccgccaa cttcacaagg tatagccagc taactagctt ggatgtagga tttaacacca | |
| 361 | tctcaaaact ggagccagaa ttgtgccaga aacttcccat gttaaaagtt ttgaacctcc | |
| 421 | agcacaatga gctatctcaa ctttctgata aaacctttgc cttctgcacg aatttgactg | |
| 481 | aactccatct catgtccaac tcaatccaga aaattaaaaa taatcccttt gtcaagcaga | |
| 541 | agaatttaat cacattagat ctgtctcata atggcttgtc atctacaaaa ttaggaactc | |
| 601 | aggttcagct ggaaaatctc caagagcttc tattatcaaa caataaaatt caagcgctaa | |
| 661 | aaagtgaaga actggatatc tttgccaatt catctttaaa aaaattagag ttgtcatcga | |
| 721 | atcaaattaa agagttttct ccagggtgtt ttcacgcaat tggaagatta tttggcctct | |
| 781 | ttctgaacaa tgtccagctg ggtcccagcc ttacagagaa gctatgtttg gaattagcaa | |
| 841 | acacaagcat tcggaatctg tctctgagta acagccagct gtccaccacc agcaatacaa | |
| 901 | ctttcttggg actaaagtgg acaaatctca ctatgctcga tctttcctac aacaacttaa | |
| 961 | atgtggttgg taacgattcc tttgcttggc ttccacaact agaatatttc ttcctagagt | |
| 1021 | ataataatat acagcatttg ttttctcact ctttgcacgg gcttttcaat gtgaggtacc | |
| 1081 | tgaatttgaa acggtctttt actaaacaaa gtatttccct tgcctcactc cccaagattg | |
| 1141 | atgatttttc ttttcagtgg ctaaaatgtt tggagcacct taacatggaa gataatgata | |
| 1201 | ttccaggcat aaaaagcaat atgttcacag gattgataaa cctgaaatac ttaagtctat | |
| 1261 | ccaactcctt tacaagtttg cgaactttga caaatgaaac atttgtatca cttgctcatt | |
| 1321 | ctcccttaca catactcaac ctaaccaaga ataaaatctc aaaaatagag agtgatgctt | |
| 1381 | tctcttggtt gggccaccta gaagtacttg acctgggcct taatgaaatt gggcaagaac | |
| 1441 | tcacaggcca ggaatggaga ggtctagaaa atattttcga aatctatctt tcctacaaca | |
| 1501 | agtacctgca gctgactagg aactcctttg ccttggtccc aagccttcaa cgactgatgc | |
| 1561 | tccgaagggt ggcccttaaa aatgtggata gctctccttc accattccag cctcttcgta | |
| 1621 | acttgaccat tctggatcta agcaacaaca acatagccaa cataaatgat gacatgttgg | |
| 1681 | agggtcttga gaaactagaa attctcgatt tgcagcataa caacttagca cggctctgga | |
| 1741 | aacacgcaaa ccctggtggt cccatttatt tcctaaaggg tctgtctcac ctccacatcc | |
| 1801 | ttaacttgga gtccaacggc tttgacgaga tcccagttga ggtcttcaag gatttatttg | |
| 1861 | aactaaagat catcgattta ggattgaata atttaaacac acttccagca tctgtcttta | |
| 1921 | ataatcaggt gtctctaaag tcattgaacc ttcagaagaa tctcataaca tccgttgaga | |
| 1981 | agaaggtttt cgggccagct ttcaggaacc tgactgagtt agatatgcgc tttaatccct | |
| 2041 | ttgattgcac gtgtgaaagt attgcctggt ttgttaattg gattaacgag acccatacca | |
| 2101 | acatccctga gctgtcaagc cactaccttt gcaacactcc acctcactat catgggttcc | |
| 2161 | cagtgagact ttttgataca tcatcttgca aagacagtgc cccctttgaa ctctttttca | |
| 2221 | tgatcaatac cagtatcctg ttgattttta tctttattgt acttctcatc cactttgagg | |
| 2281 | gctggaggat atctttttat tggaatgttt cagtacatcg agttcttggt ttcaaagaaa | |
| 2341 | tagacagaca gacagaacag tttgaatatg cagcatatat aattcatgcc tataaagata | |
| 2401 | aggattgggt ctgggaacat ttctcttcaa tggaaaagga agaccaatct ctcaaatttt | |
| 2461 | gtctggaaga aagggacttt gaggcgggtg tttttgaact agaagcaatt gttaacagca | |
| 2521 | tcaaaagaag cagaaaaatt atttttgtta taacacacca tctattaaaa gacccattat | |
| 2581 | gcaaaagatt caaggtacat catgcagttc aacaagctat tgaacaaaat ctggattcca | |
| 2641 | ttatattggt tttccttgag gagattccag attataaact gaaccatgca ctctgtttgc | |
| 2701 | gaagaggaat gtttaaatct cactgcatct tgaactggcc agttcagaaa gaacggatag | |
| 2761 | gtgcctttcg tcataaattg caagtagcac ttggatccaa aaactctgta cattaaattt | |
| 2821 | atttaaatat tcaattagca aaggagaaac tttctcaatt taaaaagttc tatggcaaat | |
| 2881 | ttaagttttc cataaaggtg ttataatttg tttattcata tttgtaaatg attatattct | |
| 2941 | atcacaatta catctcttct aggaaaatgt gtctccttat ttcaggccta tttttgacaa | |
| 3001 | ttgacttaat tttacccaaa ataaaacata taagcacgta aaaaaaaaaa aaaaaaa | |
| DNA Sequence of TLR 4, NM 138554 | | |
| 1 | tttgaataca ccaattgctg tggggcggct cgaggaagag aagacaccag tgcctcagaa | (SEQ ID NO: 3) |
| 61 | actgctcggt cagacggtga tagcgagcca cgcattcaca gggccactgc tgctcacaga | |
| 121 | agcagtgagg atgatgccag gatgatgtct gcctcgcgcc tggctgggac tctgatccca | |
| 181 | gccatggcct tcctctcctg cgtgagacca gaaagctggg agccctgcgt ggaggtggtt | |
| 241 | cctaatatta cttatcaatg catggagctg aatttctaca aaatccccga caacctcccc | |
| 301 | ttctcaacca agaacctgga cctgagcttt aatcccctga ggcatttagg cagctatagc | |
| 361 | ttcttcagtt tcccagaact gcaggtgctg gatttatcca ggtgtgaaat ccagacaatt | |
| 421 | gaagatgggg catatcagag cctaagccac ctctctacct taatattgac aggaaacccc | |
| 481 | atccagagtt tagccctggg agccttttct ggactatcaa gtttacagaa gctggtggct | |
| 541 | gtggagacaa atctagcatc tctagagaac ttccccattg gacatctcaa aactttgaaa | |
| 601 | gaacttaatg tggctcacaa tcttatccaa tctttcaaat tacctgagta tttttctaat | |
| 661 | ctgaccaatc tagagcactt ggacctttcc agcaacaaga ttcaaagtat ttattgcaca | |
| 721 | gacttgcggg ttctacatca aatgccccta ctcaatctct ctttagacct gtccctgaac | |
| 781 | cctatgaact ttatccaacc aggtgcattt aaagaaatta ggcttcataa gctgacttta | |
| 841 | agaaataatt ttgatagttt aaatgtaatg aaaacttgta ttcaaggtct ggctggttta | |
| 901 | gaagtccatc gtttggttct gggagaattt agaaatgaag gaaacttgga aaagtttgac | |
| 961 | aaatctgctc tagagggcct gtgcaatttg accattgaag aattccgatt agcatactta | |
| 1021 | gactactacc tcgatgatat tattgactta tttaattgtt tgacaaatgt ttcttcattt | |
| 1081 | tccctggtga gtgtgactat tgaaagggta aaagactttt cttataattt cggatggcaa | |
| 1141 | catttagaat tagttaactg taaatttgga cagtttccca cattgaaact caaatctctc | |
| 1201 | aaaaggctta ctttcacttc caacaaaggt gggaatgctt tttcagaagt tgatctacca | |
| 1261 | agccttgagt ttctagatct cagtagaaat ggcttgagtt tcaaaggttg ctgttctcaa | |
| 1321 | agtgattttg ggacaaccag cctaaagtat ttagatctga gcttcaatgg tgttattacc | |
| 1381 | atgagttcaa acttcttggg cttagaacaa ctagaacatc tggatttcca gcattccaat | |
| 1441 | ttgaaacaaa tgagtgagtt ttcagtattc ctatcactca gaaacctcat ttaccttgac | |
| 1501 | atttctcata ctcacaccag agttgctttc aatggcatct tcaatggctt gtccagtctc | |
| 1561 | gaagtcttga aaatggctgg caattctttc caggaaaact tccttccaga tatcttcaca | |
| 1621 | gagctgagaa acttgacctt cctggacctc tctcagtgtc aactggagca gttgtctcca | |
| 1681 | acagcattta actcactctc cagtcttcag gtactaaata tgagccacaa caacttcttt | |
| 1741 | tcattggata cgtttcctta taagtgtctg aactccctcc aggttcttga ttacagtctc | |
| 1801 | aatcacataa tgacttccaa aaaacaggaa ctacagcatt ttccaagtag tctagctttc | |
| 1861 | ttaaatctta ctcagaatga ctttgcttgt acttgtgaac accagagttt cctgcaatgg | |
| 1921 | atcaaggacc agaggcagct cttggtggaa gttgaacgaa tggaatgtgc aacaccttca | |
| 1981 | gataagcagg gcatgcctgt gctgagtttg aatatcacct gtcagatgaa taagaccatc | |
| 2041 | attggtgtgt cggtcctcag tgtgcttgta gtatctgttg tagcagttct ggtctataag | |
| 2101 | ttctattttc acctgatgct tcttgctggc tgcataaagt atggtagagg tgaaaacatc | |
| 2161 | tatgatgcct ttgttatcta ctcaagccag gatgaggact gggtaaggaa tgagctagta | |
| 2221 | aagaatttag aagaaggggt gcctccattt cagctctgcc ttcactacag agactttatt | |
| 2281 | cccggtgtgg ccattgctgc caacatcatc catgaaggtt tccataaaag ccgaaaggtg | |
| 2341 | attgttgtgg tgtcccagca cttcatccag agccgctggt gtatctttga atatgagatt | |
| 2401 | gctcagacct ggcagtttct gagcagtcgt gctggtatca tcttcattgt cctgcagaag | |
| 2461 | gtggagaaga ccctgctcag gcagcaggtg gagctgtacc gccttctcag caggaacact | |
| 2521 | tacctggagt gggaggacag tgtcctgggg cggcacatct tctggagacg actcagaaaa | |
| 2581 | gccctgctgg atggtaaatc atggaatcca gaaggaacag tgggtacagg atgcaattgg | |
| 2641 | caggaagcaa catctatctg aagaggaaaa ataaaaacct cctgaggcat ttcttgccca | |
| 2701 | gctgggtcca acacttgttc agttaataag tattaaatgc tgccacatgt caggccttat | |
| 2761 | gctaagggtg agtaattcca tggtgcacta gatatgcagg gctgctaatc tcaaggagct | |
| 2821 | tccagtgcag agggaataaa tgctagacta aaatacagag tcttccaggt gggcatttca | |
| 2881 | accaactcag tcaaggaacc catgacaaag aaagtcattt caactcttac ctcatcaagt | |
| 2941 | tgaataaaga cagagaaaac agaaagagac attgttcttt tcctgagtct tttgaatgga | |
| 3001 | aattgtatta tgttatagcc atcataaaac cattttggta gttttgactg aactgggtgt | |
| 3061 | tcactttttc ctttttgatt gaatacaatt taaattctac ttgatgactg cagtcgtcaa | |
| 3121 | ggggctcctg atgcaagatg ccccttccat tttaagtctg tctccttaca gaggttaaag | |
| 3181 | tctagtggct aattcctaag gaaacctgat taacacatgc tcacaaccat cctggtcatt | |
| 3241 | ctcgagcatg ttctattttt taactaatca cccctgatat atttttattt ttatatatcc | |
| 3301 | agttttcatt tttttacgtc ttgcctataa gctaatatca taaataaggt tgtttaagac | |
| 3361 | gtgcttcaaa tatccatatt aaccactatt tttcaaggaa gtatggaaaa gtacactctg | |
| 3421 | tcactttgtc actcgatgtc attccaaagt tattgcctac taagtaatga ctgtcatgaa | |
| 3481 | agcagcattg aaataatttg tttaaagggg gcactctttt aaacgggaag aaaatttccg | |
| 3541 | cttcctggtc ttatcatgga caatttgggc tagaggcagg aaggaagtgg gatgacctca | |
| 3601 | ggaggtcacc ttttcttgat tccagaaaca tatgggctga taaacccggg gtgacctcat | |
| 3661 | gaaatgagtt gcagcagaag tttatttttt tcagaacaag tgatgtttga tggacctctg | |
| 3721 | aatctcttta gggagacaca gatggctggg atccctcccc tgtacccttc tcactgccag | |
| 3781 | gagaactacg tgtgaaggta ttcaaggcag ggagtataca ttgctgtttc ctgttgggca | |
| 3841 | atgctccttg accacatttt gggaagagtg gatgttatca ttgagaaaac aatgtgtctg | |
| 3901 | gaattaatgg ggttcttata aagaaggttc ccagaaaaga atgttcatcc agcctcctca | |
| 3961 | gaaacagaac attcaagaaa aggacaatca ggatgtcatc agggaaatga aaataaaaac | |
| 4021 | cacaatgaga tatcacctta taccaggtag aatggctact ataaaaaaat gaagtgtcat | |
| 4081 | caaggatata gagaaattgg aacccttctt cactgctgga gggaatggaa aatggtgtag | |
| 4141 | ccgttatgaa aaacagtacg gaggtttctc aaaaattaaa aatagaactg ctatatgatc | |
| 4201 | cagcaatctc acttctgtat atatacccaa aataattgaa atcagaattt caagaaaata | |
| 4261 | tttacactcc catgttcatt gtggcactct tcacaatcac tgtttccaaa gttatggaaa | |
| 4321 | caacccaaat ttccattgaa aaataaatgg acaaagaaaa tgtgcatata cgtacaatgg | |
| 4381 | gatattattc agcctaaaaa aagggggaat cctgttattt atgacaacat gaataaaccc | |
| 4441 | ggaggccatt atgctatgta aaatgagcaa gtaacagaaa gacaaatact gcctgatttc | |
| 4501 | atttatatga ggttctaaaa tagtcaaact catagaagca gagaatagaa cagtggttcc | |
| 4561 | tagggaaaag gaggaaggga gaaatgagga aatagggagt tgtctaattg gtataaaatt | |
| 4621 | atagtatgca agatgaatta gctctaaaga tcagctgtat agcagagttc gtataatgaa | |
| 4681 | caatactgta ttatgcactt aacattttgt taagagggta cctctcatgt taagtgttct | |
| 4741 | taccatatac atatacacaa ggaagctttt ggaggtgatg gatatattta ttaccttgat | |
| 4801 | tgtggtgatg gtttgacagg tatgtgacta tgtctaaact catcaaattg tatacattaa | |
| 4861 | atatatgcag ttttataata tcaattatgt ctgaatgaag ctataaaaaa gaaaagacaa | |
| 4921 | caaaattcag ttgtcaaaac tggaaatatg accacagtca gaagtgtttg ttactgagtg | |
| 4981 | tttcagagtg tgtttggttt gagcaggtct agggtgattg aacatccctg ggtgtgtttc | |
| 5041 | catgtctcat gtactagtga aagtagatgt gtgcatttgt gcacatatcc ctatgtatcc | |
| 5101 | ctatcagggc tgtgtgtatt tgaaagtgtg tgtgtccgca tgatcatatc tgtatagaag | |
| 5161 | agagtgtgat tatatttctt gaagaataca tccatttgaa atggatgtct atggctgttt | |
| 5221 | gagatgagtt ctctactctt gtgcttgtac agtagtctcc ccttatccct tatgcttggt | |
| 5281 | ggatacgttc ttagacccca agtggatctc tgagaccgca gatggtacca aacctcatat | |
| 5341 | atgcaatatt ttttcctata cataaatacc taagataaag ttcatcttct gaattaggca | |
| 5401 | cagtaagaga ttaacaataa ctaacaataa aattgaatag ttataataat atattgtaat | |
| 5461 | aaaagttatg tgaatgtgat ctctttcttt ctctctctca aaa | |
| DNA Sequence of TLR 5, NM 003268 | | |
| 1 | ggttttcagg agcccgagcg agggcgccgc ttttgcgtcc gggaggagcc aaccgtggcg | (SEQ ID NO: 4) |
| 61 | caggcggcgc ggggaggcgt cccagagtct cactctgccg cccaggctgg actgcagtga | |
| 121 | cacaatctcg gctgactgca accactgcct ccagggttca agcgattctc ttgcctcagc | |
| 181 | ctcccaagta gctgggatta cagattgatg ttcatgttcc tgacactact acaagattca | |
| 241 | tactcctgat gctactgaca acgtggcttc tccacagtca ccaaaccagg gatgctatac | |
| 301 | tggacttccc tactctcatc tgctccagcc ccctgacctt atagttgccc agctttcctg | |
| 361 | gcaattgact ttgcccatca atacacagga tttagcatcc agggaagatg tcggagcctc | |
| 421 | agatgttaat tttctaattg agaatgttgg cgctgtccga acctggagac aggaaaacaa | |
| 481 | aaagtccttt ctcctgattc accaaaaaat aaaatactga ctaccatcac tgtgatgaga | |
| 541 | ttcctatagt ctcaggaact gaagtcttta aacaaccagg gaccctctgc ccctagaata | |
| 601 | agaacatact agaagtccct tctgctagga caacgaggat catgggagac cacctggacc | |
| 661 | ttctcctagg agtggtgctc atggccggtc ctgtgtttgg aattccttcc tgctcctttg | |
| 721 | atggccgaat agccttttat cgtttctgca acctcaccca ggtcccccag gtcctcaaca | |
| 781 | ccactgagag gctcctgctg agcttcaact atatcaggac agtcactgct tcatccttcc | |
| 841 | cctttctgga acagctgcag ctgctggagc tcgggagcca gtataccccc ttgactattg | |
| 901 | acaaggaggc cttcagaaac ctgcccaacc ttagaatctt ggacctggga agtagtaaga | |
| 961 | tatacttctt gcatccagat gcttttcagg gactgttcca tctgtttgaa cttagactgt | |
| 1021 | atttctgtgg tctctctgat gctgtattga aagatggtta tttcagaaat ttaaaggctt | |
| 1081 | taactcgctt ggatctatcc aaaaatcaga ttcgtagcct ttaccttcat ccttcatttg | |
| 1141 | ggaagttgaa ttccttaaag tccatagatt tttcctccaa ccaaatattc cttgtatgtg | |
| 1201 | aacatgagct cgagccccta caagggaaaa cgctctcctt ttttagcctc gcagctaata | |
| 1261 | gcttgtatag cagagtctca gtggactggg gaaaatgtat gaacccattc agaaacatgg | |
| 1321 | tgctggagat actagatgtt tctggaaatg gctggacagt ggacatcaca ggaaacttta | |
| 1381 | gcaatgccat cagcaaaagc caggccttct ctttgattct tgcccaccac atcatgggtg | |
| 1441 | ccgggtttgg cttccataac atcaaagatc ctgaccagaa cacatttgct ggcctggcca | |
| 1501 | gaagttcagt gagacacctg gatctttcac atgggtttgt cttctccctg aactcacgag | |
| 1561 | tctttgagac actcaaggat ttgaaggttc tgaaccttgc ctacaacaag ataaataaga | |
| 1621 | ttgcagatga agcattttac ggacttgaca acctccaagt tctcaatttg tcatataacc | |
| 1681 | ttctggggga actttacagt tcgaatttct atggactacc taaggtagcc tacattgatt | |
| 1741 | tgcaaaagaa tcacattgca ataattcaag accaaacatt caaattcctg gaaaaattac | |
| 1801 | agaccttgga tctccgagac aatgctctta caaccattca ttttattcca agcatacccg | |
| 1861 | atatcttctt gagtggcaat aaactagtga ctttgccaaa gatcaacctt acagcgaacc | |
| 1921 | tcatccactt atcagaaaac aggctagaaa atctagatat tctctacttt ctcctacggg | |
| 1981 | tacctcatct ccagattctc attttaaatc aaaatcgctt ctcctcctgt agtggagatc | |
| 2041 | aaaccccttc agagaatccc agcttagaac agcttttcct tggagaaaat atgttgcaac | |
| 2101 | ttgcctggga aactgagctc tgttgggatg tttttgaggg actttctcat cttcaagttc | |
| 2161 | tgtatttgaa tcataactat cttaattccc ttccaccagg agtatttagc catctgactg | |
| 2221 | cattaagggg actaagcctc aactccaaca ggctgacagt tctttctcac aatgatttac | |
| 2281 | ctgctaattt agagatcctg gacatatcca ggaaccagct cctagctcct aatcctgatg | |
| 2341 | tatttgtatc acttagtgtc ttggatataa ctcataacaa gttcatttgt gaatgtgaac | |
| 2401 | ttagcacttt tatcaattgg cttaatcaca ccaatgtcac tatagctggg cctcctgcag | |
| 2461 | acatatattg tgtgtaccct gactcgttct ctggggtttc cctcttctct ctttccacgg | |
| 2521 | aaggttgtga tgaagaggaa gtcttaaagt ccctaaagtt ctcccttttc attgtatgca | |
| 2581 | ctgtcactct gactctgttc ctcatgacca tcctcacagt cacaaagttc cggggcttct | |
| 2641 | gttttatctg ttataagaca gcccagagac tggtgttcaa ggaccatccc cagggcacag | |
| 2701 | aacctgatat gtacaaatat gatgcctatt tgtgcttcag cagcaaagac ttcacatggg | |
| 2761 | tgcagaatgc tttgctcaaa cacctggaca ctcaatacag tgaccaaaac agattcaacc | |
| 2821 | tgtgctttga agaaagagac tttgtcccag gagaaaaccg cattgccaat atccaggatg | |
| 2881 | ccatctggaa cagtagaaag atcgtttgtc ttgtgagcag acacttcctt agagatggct | |
| 2941 | ggtgccttga agccttcagt tatgcccagg gcaggtgctt atctgacctt aacagtgctc | |
| 3001 | tcatcatggt ggtggttggg tccttgtccc agtaccagtt gatgaaacat caatccatca | |
| 3061 | gaggctttgt acagaaacag cagtatttga ggtggcctga ggatctccag gatgttggct | |
| 3121 | ggtttcttca taaactctct caacagatac taaagaaaga aaaagaaaag aagaaagaca | |
| 3181 | ataacattcc gttgcaaact gtagcaacca tctcctaatc aaaggagcaa tttccaactt | |
| 3241 | atctcaagcc acaaataact cttcactttg tatttgcacc aagttatcat tttggggtcc | |
| 3301 | tctctggagg tttttttttt ctttttgcta ctatgaaaac aacataaatc tctcaatttt | |
| 3361 | cgtatcaaca ccatgttctg tctcactaac ctccaaatgg aaaataatag atctagaaaa | |
| 3421 | ttgcaactgc c | |
| DNA Sequence of TLR 7, NMK 016562 | | |
| 1 | gaagactcca gatataggat cactccatgc catcaagaaa gttgatgcta ttgggcccat | (SEQ ID NO: 5) |
| 61 | ctcaagctga tcttggcacc tctcatgctc tgctctcttc aaccagacct ctacattcca | |
| 121 | ttttggaaga agactaaaaa tggtgtttcc aatgtggaca ctgaagagac aaattcttat | |
| 181 | cctttttaac ataatcctaa tttccaaact ccttggggct agatggtttc ctaaaactct | |
| 241 | gccctgtgat gtcactctgg atgttccaaa gaaccatgtg atcgtggact gcacagacaa | |
| 301 | gcatttgaca gaaattcctg gaggtattcc cacgaacacc acgaacctca ccctcaccat | |
| 361 | taaccacata ccagacatct ccccagcgtc ctttcacaga ctggaccatc tggtagagat | |
| 421 | cgatttcaga tgcaactgtg tacctattcc actggggtca aaaaacaaca tgtgcatcaa | |
| 481 | gaggctgcag attaaaccca gaagctttag tggactcact tatttaaaat ccctttacct | |
| 541 | ggatggaaac cagctactag agataccgca gggcctcccg cctagcttac agcttctcag | |
| 601 | ccttgaggcc aacaacatct tttccatcag aaaagagaat ctaacagaac tggccaacat | |
| 661 | agaaatactc tacctgggcc aaaactgtta ttatcgaaat ccttgttatg tttcatattc | |
| 721 | aatagagaaa gatgccttcc taaacttgac aaagttaaaa gtgctctccc tgaaagataa | |
| 781 | caatgtcaca gccgtcccta ctgttttgcc atctacttta acagaactat atctctacaa | |
| 841 | caacatgatt gcaaaaatcc aagaagatga ttttaataac ctcaaccaat tacaaattct | |
| 901 | tgacctaagt ggaaattgcc ctcgttgtta taatgcccca tttccttgtg cgccgtgtaa | |
| 961 | aaataattct cccctacaga tccctgtaaa tgcttttgat gcgctgacag aattaaaagt | |
| 1021 | tttacgtcta cacagtaact ctcttcagca tgtgccccca agatggttta agaacatcaa | |
| 1081 | caaactccag gaactggatc tgtcccaaaa cttcttggcc aaagaaattg gggatgctaa | |
| 1141 | atttctgcat tttctcccca gcctcatcca attggatctg tctttcaatt ttgaacttca | |
| 1201 | ggtctatcgt gcatctatga atctatcaca agcattttct tcactgaaaa gcctgaaaat | |
| 1261 | tctgcggatc agaggatatg tctttaaaga gttgaaaagc tttaacctct cgccattaca | |
| 1321 | taatcttcaa aatcttgaag ttcttgatct tggcactaac tttataaaaa ttgctaacct | |
| 1381 | cagcatgttt aaacaattta aaagactgaa agtcatagat ctttcagtga ataaaatatc | |
| 1441 | accttcagga gattcaagtg aagttggctt ctgctcaaat gccagaactt ctgtagaaag | |
| 1501 | ttatgaaccc caggtcctgg aacaattaca ttatttcaga tatgataagt atgcaaggag | |
| 1561 | ttgcagattc aaaaacaaag aggcttcttt catgtctgtt aatgaaagct gctacaagta | |
| 1621 | tgggcagacc ttggatctaa gtaaaaatag tatatttttt gtcaagtcct ctgattttca | |
| 1681 | gcatctttct ttcctcaaat gcctgaatct gtcaggaaat ctcattagcc aaactcttaa | |
| 1741 | tggcagtgaa ttccaacctt tagcagagct gagatatttg gacttctcca acaaccggct | |
| 1801 | tgatttactc cattcaacag catttgaaga gcttcacaaa ctggaagttc tggatataag | |
| 1861 | cagtaatagc cattattttc aatcagaagg aattactcat atgctaaact ttaccaagaa | |
| 1921 | cctaaaggtt ctgcagaaac tgatgatgaa cgacaatgac atctcttcct ccaccagcag | |
| 1981 | gaccatggag agtgagtctc ttagaactct ggaattcaga ggaaatcact tagatgtttt | |
| 2041 | atggagagaa ggtgataaca gatacttaca attattcaag aatctgctaa aattagagga | |
| 2101 | attagacatc tctaaaaatt ccctaagttt cttgccttct ggagtttttg atggtatgcc | |
| 2161 | tccaaatcta aagaatctct ctttggccaa aaatgggctc aaatctttca gttggaagaa | |
| 2221 | actccagtgt ctaaagaacc tggaaacttt ggacctcagc cacaaccaac tgaccactgt | |
| 2281 | ccctgagaga ttatccaact gttccagaag cctcaagaat ctgattctta agaataatca | |
| 2341 | aatcaggagt ctgacgaagt attttctaca agatgccttc cagttgcgat atctggatct | |
| 2401 | cagctcaaat aaaatccaga tgatccaaaa gaccagcttc ccagaaaatg tcctcaacaa | |
| 2461 | tctgaagatg ttgcttttgc atcataatcg gtttctgtgc acctgtgatg ctgtgtggtt | |
| 2521 | tgtctggtgg gttaaccata cggaggtgac tattccttac ctggccacag atgtgacttg | |
| 2581 | tgtggggcca ggagcacaca agggccaaag tgtgatctcc ctggatctgt acacctgtga | |
| 2641 | gttagatctg actaacctga ttctgttctc actttccata tctgtatctc tctttctcat | |
| 2701 | ggtgatgatg acagcaagtc acctctattt ctgggatgtg tggtatattt accatttctg | |
| 2761 | taaggccaag ataaaggggt atcagcgtct aatatcacca gactgttgct atgatgcttt | |
| 2821 | tattgtgtat gacactaaag acccagctgt gaccgagtgg gttttggctg agctggtggc | |
| 2881 | caaactggaa gacccaagag agaaacattt taatttatgt ctcgaggaaa gggactggtt | |
| 2941 | accagggcag ccagttctgg aaaacctttc ccagagcata cagcttagca aaaagacagt | |
| 3001 | gtttgtgatg acagacaagt atgcaaagac tgaaaatttt aagatagcat tttacttgtc | |
| 3061 | ccatcagagg ctcatggatg aaaaagttga tgtgattatc ttgatatttc ttgagaagcc | |
| 3121 | ctttcagaag tccaagttcc tccagctccg gaaaaggctc tgtgggagtt ctgtccttga | |
| 3181 | gtggccaaca aacccgcaag ctcacccata cttctggcag tgtctaaaga acgccctggc | |
| 3241 | cacagacaat catgtggcct atagtcaggt gttcaaggaa acggtctagc ccttctttgc | |
| 3301 | aaaacacaac tgcctagttt accaaggaga ggcctggctg tttaaattgt tttcatatat | |
| 3361 | atcacaccaa aagcgtgttt tgaaattctt caagaaatga gattgcccat atttcagggg | |
| 3421 | agccaccaac gtctgtcaca ggagttggaa agatggggtt tatataatgc atcaagtctt | |
| 3481 | ctttcttatc tctctgtgtc tctatttgca cttgagtctc tcacctcagc tcctgtaaaa | |
| 3541 | gagtggcaag taaaaaacat ggggctctga ttctcctgta attgtgataa ttaaatatac | |
| 3601 | acacaatcat gacattgaga agaactgcat ttctaccctt aaaaagtact ggtatataca | |
| 3661 | gaaatagggt taaaaaaaac tcaagctctc tctatatgag accaaaatgt actagagtta | |
| 3721 | gtttagtgaa ataaaaaacc agtcagctgg ccgggcatgg tggctcatgc ttgtaatccc | |
| 3781 | agcactttgg gaggccgagg caggtggatc acgaggtcag gagtttgaga ccagtctggc | |
| 3841 | caacatggtg aaaccccgtc tgtactaaaa atacaaaaat tagctgggcg tggtggtggg | |
| 3901 | tgcctgtaat cccagctact tgggaggctg aggcaggaga atcgcttgaa cccgggaggt | |
| 3961 | ggaggtggca gtgagccgag atcacgccac tgcaatgcag cccgggcaac agagctagac | |
| 4021 | tgtctcaaaa gaacaaaaaa aaaaaaacac aaaaaaactc agtcagcttc ttaaccaatt | |
| 4081 | gcttccgtgt catccagggc cccattctgt gcagattgag tgtgggcacc acacaggtgg | |
| 4141 | ttgctgcttc agtgcttcct gctctttttc cttgggcctg cttctgggtt ccatagggaa | |
| 4201 | acagtaagaa agaaagacac atccttacca taaatgcata tggtccacct acaaatagaa | |
| 4261 | aaatatttaa atgatctgcc tttatacaaa gtgatattct ctacctttga taatttacct | |
| 4321 | gcttaaatgt ttttatctgc actgcaaagt actgtatcca aagtaaaatt tcctcatcca | |
| 4381 | atatctttca aactgttttg ttaactaatg ccatatattt gtaagtatct gcacacttga | |
| 4441 | tacagcaacg ttagatggtt ttgatggtaa accctaaagg aggactccaa gagtgtgtat | |
| 4501 | ttatttatag ttttatcaga gatgacaatt atttgaatgc caattatatg gattcctttc | |
| 4561 | attttttgct ggaggatggg agaagaaacc aaagtttata gaccttcaca ttgagaaagc | |
| 4621 | ttcagttttg aacttcagct atcagattca aaaacaacag aaagaaccaa gacattctta | |
| 4681 | agatgcctgt actttcagct gggtataaat tcatgagttc aaagattgaa acctgaccaa | |
| 4741 | tttgctttat ttcatggaag aagtgatcta caaaggtgtt tgtgccattt ggaaaacagc | |
| 4801 | gtgcatgtgt tcaagcctta gattggcgat gtcgtatttt cctcacgtgt ggcaatgcca | |
| 4861 | aaggctttac tttacctgtg agtacacact atatgaatta tttccaacgt acatttaatc | |
| 4921 | aataagggtc acaaattccc aaatcaatct ctggaataaa tagagaggta attaaattgc | |
| 4981 | tggagccaac ta | |
| DNA sequence of TLR 8, NM 138636 | | |
| 1 | ctcctgcata gagggtacca ttctgcgctg ctgcaagtta cggaatgaaa aattagaaca | (SEQ ID NO: 6) |
| 61 | acagaaacat ggaaaacatg ttccttcagt cgtcaatgct gacctgcatt ttcctgctaa | |
| 121 | tatctggttc ctgtgagtta tgcgccgaag aaaatttttc tagaagctat ccttgtgatg | |
| 181 | agaaaaagca aaatgactca gttattgcag agtgcagcaa tcgtcgacta caggaagttc | |
| 241 | cccaaacggt gggcaaatat gtgacagaac tagacctgtc tgataatttc atcacacaca | |
| 301 | taacgaatga atcatttcaa gggctgcaaa atctcactaa aataaatcta aaccacaacc | |
| 361 | ccaatgtaca gcaccagaac ggaaatcccg gtatacaatc aaatggcttg aatatcacag | |
| 421 | acggggcatt cctcaaccta aaaaacctaa gggagttact gcttgaagac aaccagttac | |
| 481 | cccaaatacc ctctggtttg ccagagtctt tgacagaact tagtctaatt caaaacaata | |
| 541 | tatacaacat aactaaagag ggcatttcaa gacttataaa cttgaaaaat ctctatttgg | |
| 601 | cctggaactg ctattttaac aaagtttgcg agaaaactaa catagaagat ggagtatttg | |
| 661 | aaacgctgac aaatttggag ttgctatcac tatctttcaa ttctctttca cacgtgccac | |
| 721 | ccaaactgcc aagctcccta cgcaaacttt ttctgagcaa cacccagatc aaatacatta | |
| 781 | gtgaagaaga tttcaaggga ttgataaatt taacattact agatttaagc gggaactgtc | |
| 841 | cgaggtgctt caatgcccca tttccatgcg tgccttgtga tggtggtgct tcaattaata | |
| 901 | tagatcgttt tgcttttcaa aacttgaccc aacttcgata cctaaacctc tctagcactt | |
| 961 | ccctcaggaa gattaatgct gcctggttta aaaatatgcc tcatctgaag gtgctggatc | |
| 1021 | ttgaattcaa ctatttagtg ggagaaatag cctctggggc atttttaacg atgctgcccc | |
| 1081 | gcttagaaat acttgacttg tcttttaact atataaaggg gagttatcca cagcatatta | |
| 1141 | atatttccag aaacttctct aaacttttgt ctctacgggc attgcattta agaggttatg | |
| 1201 | tgttccagga actcagagaa gatgatttcc agcccctgat gcagcttcca aacttatcga | |
| 1261 | ctatcaactt gggtattaat tttattaagc aaatcgattt caaacttttc caaaatttct | |
| 1321 | ccaatctgga aattatttac ttgtcagaaa acagaatatc accgttggta aaagataccc | |
| 1381 | ggcagagtta tgcaaatagt tcctcttttc aacgtcatat ccggaaacga cgctcaacag | |
| 1441 | attttgagtt tgacccacat tcgaactttt atcatttcac ccgtccttta ataaagccac | |
| 1501 | aatgtgctgc ttatggaaaa gccttagatt taagcctcaa cagtattttc ttcattgggc | |
| 1561 | caaaccaatt tgaaaatctt cctgacattg cctgtttaaa tctgtctgca aatagcaatg | |
| 1621 | ctcaagtgtt aagtggaact gaattttcag ccattcctca tgtcaaatat ttggatttga | |
| 1681 | caaacaatag actagacttt gataatgcta gtgctcttac tgaattgtcc gacttggaag | |
| 1741 | ttctagatct cagctataat tcacactatt tcagaatagc aggcgtaaca catcatctag | |
| 1801 | aatttattca aaatttcaca aatctaaaag ttttaaactt gagccacaac aacatttata | |
| 1861 | ctttaacaga taagtataac ctggaaagca agtccctggt agaattagtt ttcagtggca | |
| 1921 | atcgccttga cattttgtgg aatgatgatg acaacaggta tatctccatt ttcaaaggtc | |
| 1981 | tcaagaatct gacacgtctg gatttatccc ttaataggct gaagcacatc ccaaatgaag | |
| 2041 | cattccttaa tttgccagcg agtctcactg aactacatat aaatgataat atgttaaagt | |
| 2101 | tttttaactg gacattactc cagcagtttc ctcgtctcga gttgcttgac ttacgtggaa | |
| 2161 | acaaactact ctttttaact gatagcctat ctgactttac atcttccctt cggacactgc | |
| 2221 | tgctgagtca taacaggatt tcccacctac cctctggctt tctttctgaa gtcagtagtc | |
| 2281 | tgaagcacct cgatttaagt tccaatctgc taaaaacaat caacaaatcc gcacttgaaa | |
| 2341 | ctaagaccac caccaaatta tctatgttgg aactacacgg aaaccccttt gaatgcacct | |
| 2401 | gtgacattgg agatttccga agatggatgg atgaacatct gaatgtcaaa attcccagac | |
| 2461 | tggtagatgt catttgtgcc agtcctgggg atcaaagagg gaagagtatt gtgagtctgg | |
| 2521 | agctaacaac ttgtgtttca gatgtcactg cagtgatatt atttttcttc acgttcttta | |
| 2581 | tcaccaccat ggttatgttg gctgccctgg ctcaccattt gttttactgg gatgtttggt | |
| 2641 | ttatatataa tgtgtgttta gctaaggtaa aaggctacag gtctctttcc acatcccaaa | |
| 2701 | ctttctatga tgcttacatt tcttatgaca ccaaagatgc ctctgttact gactgggtga | |
| 2761 | taaatgagct gcgctaccac cttgaagaga gccgagacaa aaacgttctc ctttgtctag | |
| 2821 | aggagaggga ttgggatccg ggattggcca tcatcgacaa cctcatgcag agcatcaacc | |
| 2881 | aaagcaagaa aacagtattt gttttaacca aaaaatatgc aaaaagctgg aactttaaaa | |
| 2941 | cagcttttta cttggctttg cagaggctaa tggatgagaa catggatgtg attatattta | |
| 3001 | tcctgctgga gccagtgtta cagcattctc agtatttgag gctacggcag cggatctgta | |
| 3061 | agagctccat cctccagtgg cctgacaacc cgaaggcaga aggcttgttt tggcaaactc | |
| 3121 | tgagaaatgt ggtcttgact gaaaatgatt cacggtataa caatatgtat gtcgattcca | |
| 3181 | ttaagcaata ctaactgacg ttaagtcatg atttcgcgcc ataataaaga tgcaaaggaa | |
| 3241 | tgacatttct gtattagtta tctattgcta tgtaacaaat tatcccaaaa cttagtggtt | |
| 3301 | taaaacaaca catttgctgg cccacagttt ttgagggtca ggagtccagg cccagcataa | |
| 3361 | ctgggtcctc tgctcagggt gtctcagagg ctgcaatgta ggtgttcacc agagacatag | |
| 3421 | gcatcactgg ggtcacactc atgtggttgt tttctggatt caattcctcc tgggctattg | |
| 3481 | gccaaaggct atactcatgt aagccatgcg agcctctccc acaaggcagc ttgcttcatc | |
| 3541 | agagctagca aaaaagagag gttgctagca agatgaagtc acaatctttt gtaatcgaat | |
| 3601 | caaaaaagtg atatctcatc actttggcca tattctattt gttagaagta aaccacaggt | |
| 3661 | cccaccagct ccatgggagt gaccacctca gtccagggaa aacagctgaa gaccaagatg | |
| 3721 | gtgagctctg attgcttcag ttggtcatca actattttcc cttgactgct gtcctgggat | |
| 3781 | ggcctgctat cttgatgata gattgtgaat atcaggaggc agggatcact gtggaccatc | |
| 3841 | ttagcagttg acctaacaca tcttcttttc aatatctaag aacttttgcc actgtgacta | |
| 3901 | atggtcctaa tattaagctg ttgtttatat ttatcatata tctatggcta catggttata | |
| 3961 | ttatgctgtg gttgcgttcg gttttattta cagttgcttt tacaaatatt tgctgtaaca | |
| 4021 | tttgacttct aaggtttaga tgccatttaa gaactgagat ggatagcttt taaagcatct | |
| 4081 | tttacttctt accatttttt aaaagtatgc agctaaattc gaagcttttg gtctatattg | |
| 4141 | ttaattgcca ttgctgtaaa tcttaaaatg aatgaataaa aatgtttcat tttacaa | |
| DNA Sequence of TLR 9, NM 017442 | | |
| 1 | ggaggtcttg tttccggaag atgttgcaag gctgtggtga aggcaggtgc agcctagcct | (SEQ ID NO: 7) |
| 61 | cctgctcaag ctacaccctg gccctccacg catgaggccc tgcagaactc tggagatggt | |
| 121 | gcctacaagg gcagaaaagg acaagtcggc agccgctgtc ctgagggcac cagctgtggt | |
| 181 | gcaggagcca agacctgagg gtggaagtgt cctcttagaa tggggagtgc ccagcaaggt | |
| 241 | gtacccgcta ctggtgctat ccagaattcc catctctccc tgctctctgc ctgagctctg | |
| 301 | ggccttagct cctccctggg cttggtagag gacaggtgtg aggccctcat gggatgtagg | |
| 361 | ctgtctgaga ggggagtgga aagaggaagg ggtgaaggag ctgtctgcca tttgactatg | |
| 421 | caaatggcct ttgactcatg ggaccctgtc ctcctcactg ggggcagggt ggagtggagg | |
| 481 | gggagctact aggctggtat aaaaatctta cttcctctat tctctgagcc gctgctgccc | |
| 541 | ctgtgggaag ggacctcgag tgtgaagcat ccttccctgt agctgctgtc cagtctgccc | |
| 601 | gccagaccct ctggagaagc ccctgccccc cagcatgggt ttctgccgca gcgccctgca | |
| 661 | cccgctgtct ctcctggtgc aggccatcat gctggccatg accctggccc tgggtacctt | |
| 721 | gcctgccttc ctaccctgtg agctccagcc ccacggcctg gtgaactgca actggctgtt | |
| 781 | cctgaagtct gtgccccact tctccatggc agcaccccgt ggcaatgtca ccagcctttc | |
| 841 | cttgtcctcc aaccgcatcc accacctcca tgattctgac tttgcccacc tgcccagcct | |
| 901 | gcggcatctc aacctcaagt ggaactgccc gccggttggc ctcagcccca tgcacttccc | |
| 961 | ctgccacatg accatcgagc ccagcacctt cttggctgtg cccaccctgg aagagctaaa | |
| 1021 | cctgagctac aacaacatca tgactgtgcc tgcgctgccc aaatccctca tatccctgtc | |
| 1081 | cctcagccat accaacatcc tgatgctaga ctctgccagc ctcgccggcc tgcatgccct | |
| 1141 | gcgcttccta ttcatggacg gcaactgtta ttacaagaac ccctgcaggc aggcactgga | |
| 1201 | ggtggccccg ggtgccctcc ttggcctggg caacctcacc cacctgtcac tcaagtacaa | |
| 1261 | caacctcact gtggtgcccc gcaacctgcc ttccagcctg gagtatctgc tgttgtccta | |
| 1321 | caaccgcatc gtcaaactgg cgcctgagga cctggccaat ctgaccgccc tgcgtgtgct | |
| 1381 | cgatgtgggc ggaaattgcc gccgctgcga ccacgctccc aacccctgca tggagtgccc | |
| 1441 | tcgtcacttc ccccagctac atcccgatac cttcagccac ctgagccgtc ttgaaggcct | |
| 1501 | ggtgttgaag gacagttctc tctcctggct gaatgccagt tggttccgtg ggctgggaaa | |
| 1561 | cctccgagtg ctggacctga gtgagaactt cctctacaaa tgcatcacta aaaccaaggc | |
| 1621 | cttccagggc ctaacacagc tgcgcaagct taacctgtcc ttcaattacc aaaagagggt | |
| 1681 | gtcctttgcc cacctgtctc tggccccttc cttcgggagc ctggtcgccc tgaaggagct | |
| 1741 | ggacatgcac ggcatcttct tccgctcact cgatgagacc acgctccggc cactggcccg | |
| 1801 | cctgcccatg ctccagactc tgcgtctgca gatgaacttc atcaaccagg cccagctcgg | |
| 1861 | catcttcagg gccttccctg gcctgcgcta cgtggacctg tcggacaacc gcatcagcgg | |
| 1921 | agcttcggag ctgacagcca ccatggggga ggcagatgga ggggagaagg tctggctgca | |
| 1981 | gcctggggac cttgctccgg ccccagtgga cactcccagc tctgaagact tcaggcccaa | |
| 2041 | ctgcagcacc ctcaacttca ccttggatct gtcacggaac aacctggtga ccgtgcagcc | |
| 2101 | ggagatgttt gcccagctct cgcacctgca gtgcctgcgc ctgagccaca actgcatctc | |
| 2161 | gcaggcagtc aatggctccc agttcctgcc gctgaccggt ctgcaggtgc tagacctgtc | |
| 2221 | ccacaataag ctggacctct accacgagca ctcattcacg gagctaccac gactggaggc | |
| 2281 | cctggacctc agctacaaca gccagccctt tggcatgcag ggcgtgggcc acaacttcag | |
| 2341 | cttcgtggct cacctgcgca ccctgcgcca cctcagcctg gcccacaaca acatccacag | |
| 2401 | ccaagtgtcc cagcagctct gcagtacgtc gctgcgggcc ctggacttca gcggcaatgc | |
| 2461 | actgggccat atgtgggccg agggagacct ctatctgcac ttcttccaag gcctgagcgg | |
| 2521 | tttgatctgg ctggacttgt cccagaaccg cctgcacacc ctcctgcccc aaaccctgcg | |
| 2581 | caacctcccc aagagcctac aggtgctgcg tctccgtgac aattacctgg ccttctttaa | |
| 2641 | gtggtggagc ctccacttcc tgcccaaact ggaagtcctc gacctggcag gaaaccagct | |
| 2701 | gaaggccctg accaatggca gcctgcctgc tggcacccgg ctccggaggc tggatgtcag | |
| 2761 | ctgcaacagc atcagcttcg tggcccccgg cttcttttcc aaggccaagg agctgcgaga | |
| 2821 | gctcaacctt agcgccaacg ccctcaagac agtggaccac tcctggtttg ggcccctggc | |
| 2881 | gagtgccctg caaatactag atgtaagcgc caaccctctg cactgcgcct gtggggcggc | |
| 2941 | ctttatggac ttcctgctgg aggtgcaggc tgccgtgccc ggtctgccca gccgggtgaa | |
| 3001 | gtgtggcagt ccgggccagc tccagggcct cagcatcttt gcacaggacc tgcgcctctg | |
| 3061 | cctggatgag gccctctcct gggactgttt cgccctctcg ctgctggctg tggctctggg | |
| 3121 | cctgggtgtg cccatgctgc atcacctctg tggctgggac ctctggtact gcttccacct | |
| 3181 | gtgcctggcc tggcttccct ggcgggggcg gcaaagtggg cgagatgagg atgccctgcc | |
| 3241 | ctacgatgcc ttcgtggtct tcgacaaaac gcagagcgca gtggcagact gggtgtacaa | |
| 3301 | cgagcttcgg gggcagctgg aggagtgccg tgggcgctgg gcactccgcc tgtgcctgga | |
| 3361 | ggaacgcgac tggctgcctg gcaaaaccct ctttgagaac ctgtgggcct cggtctatgg | |
| 3421 | cagccgcaag acgctgtttg tgctggccca cacggaccgg gtcagtggtc tcttgcgcgc | |
| 3481 | cagcttcctg ctggcccagc agcgcctgct ggaggaccgc aaggacgtcg tggtgctggt | |
| 3541 | gatcctgagc cctgacggcc gccgctcccg ctatgtgcgg ctgcgccagc gcctctgccg | |
| 3601 | ccagagtgtc ctcctctggc cccaccagcc cagtggtcag cgcagcttct gggcccagct | |
| 3661 | gggcatggcc ctgaccaggg acaaccacca cttctataac cggaacttct gccagggacc | |
| 3721 | cacggccgaa tagccgtgag ccggaatcct gcacggtgcc acctccacac tcacctcacc | |
| 3781 | tctgcctgcc tggtctgacc ctcccctgct cgcctccctc accccacacc tgacacagag | |
| 3841 | caggcactca ataaatgcta ccgaaggc | |
| DNA Sequence of TLR 10, NM 030956 | | |
| 1 | gaatcatcca cgcacctgca gctctgctga gagagtgcaa gccgtgggaa ttcagcagct | (SEQ ID NO: 8) |
| 61 | gaatatcaag acctttgaat tcaacaagaa gttaagacat ttatagttgt ctaacaacag | |
| 121 | actgaagatt gtggcttggt attcactggc aggtttcaga catttagatc tttcttttaa | |
| 181 | tgactaacac catgcctatc tgtggagaag ctggcaacat gtcacacctg gaaattgttt | |
| 241 | ttcaacatta atactattat ttggcagtaa tccagattgc ttttgccacc aacctgaaga | |
| 301 | catatagagg cagaaggaca ggaataattc tatttgtttc ctgttttgaa acttccatct | |
| 361 | gtaaggctat caaaaggaga tgtgagagag ggtattgagt ctggcctgac aatgcagttc | |
| 421 | ttaaaccaaa ggtccattat gcttctcctc tctgagaatc ctgacttacc tcaacaacgg | |
| 481 | agacatggca cagtagccag cttggagact tctcagccaa tgctctgaga tcaagtcgaa | |
| 541 | gacccaatat acagggtttt gagctcatct tcatcattca tatgaggaaa taagtggtaa | |
| 601 | aatccttgga aatacaatga gactcatcag aaacatttac atattttgta gtattgttat | |
| 661 | gacagcagag ggtgatgctc cagagctgcc agaagaaagg gaactgatga ccaactgctc | |
| 721 | caacatgtct ctaagaaagg ttcccgcaga cttgacccca gccacaacga cactggattt | |
| 781 | atcctataac ctcctttttc aactccagag ttcagatttt cattctgtct ccaaactgag | |
| 841 | agttttgatt ctatgccata acagaattca acagctggat ctcaaaacct ttgaattcaa | |
| 901 | caaggagtta agatatttag atttgtctaa taacagactg aagagtgtaa cttggtattt | |
| 961 | actggcaggt ctcaggtatt tagatctttc ttttaatgac tttgacacca tgcctatctg | |
| 1021 | tgaggaagct ggcaacatgt cacacctgga aatcctaggt ttgagtgggg caaaaataca | |
| 1081 | aaaatcagat ttccagaaaa ttgctcatct gcatctaaat actgtcttct taggattcag | |
| 1141 | aactcttcct cattatgaag aaggtagcct gcccatctta aacacaacaa aactgcacat | |
| 1201 | tgttttacca atggacacaa atttctgggt tcttttgcgt gatggaatca agacttcaaa | |
| 1261 | aatattagaa atgacaaata tagatggcaa aagccaattt gtaagttatg aaatgcaacg | |
| 1321 | aaatcttagt ttagaaaatg ctaagacatc ggttctattg cttaataaag ttgatttact | |
| 1381 | ctgggacgac cttttcctta tcttacaatt tgtttggcat acatcagtgg aacactttca | |
| 1441 | gatccgaaat gtgacttttg gtggtaaggc ttatcttgac cacaattcat ttgactactc | |
| 1501 | aaatactgta atgagaacta taaaattgga gcatgtacat ttcagagtgt tttacattca | |
| 1561 | acaggataaa atctatttgc ttttgaccaa aatggacata gaaaacctga caatatcaaa | |
| 1621 | tgcacaaatg ccacacatgc ttttcccgaa ttatcctacg aaattccaat atttaaattt | |
| 1681 | tgccaataat atcttaacag acgagttgtt taaaagaact atccaactgc ctcacttgaa | |
| 1741 | aactctcatt ttgaatggca ataaactgga gacactttct ttagtaagtt gctttgctaa | |
| 1801 | caacacaccc ttggaacact tggatctgag tcaaaatcta ttacaacata aaaatgatga | |
| 1861 | aaattgctca tggccagaaa ctgtggtcaa tatgaatctg tcatacaata aattgtctga | |
| 1921 | ttctgtcttc aggtgcttgc ccaaaagtat tcaaatactt gacctaaata ataaccaaat | |
| 1981 | ccaaactgta cctaaagaga ctattcatct gatggcctta cgagaactaa atattgcatt | |
| 2041 | taattttcta actgatctcc ctggatgcag tcatttcagt agactttcag ttctgaacat | |
| 2101 | tgaaatgaac ttcattctca gcccatctct ggattttgtt cagagctgcc aggaagttaa | |
| 2161 | aactctaaat gcgggaagaa atccattccg gtgtacctgt gaattaaaaa atttcattca | |
| 2221 | gcttgaaaca tattcagagg tcatgatggt tggatggtca gattcataca cctgtgaata | |
| 2281 | ccctttaaac ctaaggggaa ctaggttaaa agacgttcat ctccacgaat tatcttgcaa | |
| 2341 | cacagctctg ttgattgtca ccattgtggt tattatgcta gttctggggt tggctgtggc | |
| 2401 | cttctgctgt ctccactttg atctgccctg gtatctcagg atgctaggtc aatgcacaca | |
| 2461 | aacatggcac agggttagga aaacaaccca agaacaactc aagagaaatg tccgattcca | |
| 2521 | cgcatttatt tcatacagtg aacatgattc tctgtgggtg aagaatgaat tgatccccaa | |
| 2581 | tctagagaag gaagatggtt ctatcttgat ttgcctttat gaaagctact ttgaccctgg | |
| 2641 | caaaagcatt agtgaaaata ttgtaagctt cattgagaaa agctataagt ccatctttgt | |
| 2701 | tttgtctccc aactttgtcc agaatgagtg gtgccattat gaattctact ttgcccacca | |
| 2761 | caatctcttc catgaaaatt ctgatcatat aattcttatc ttactggaac ccattccatt | |
| 2821 | ctattgcatt cccaccaggt atcataaact gaaagctctc ctggaaaaaa aagcatactt | |
| 2881 | ggaatggccc aaggataggc gtaaatgtgg gcttttctgg gcaaaccttc gagctgctat | |
| 2941 | taatgttaat gtattagcca ccagagaaat gtatgaactg cagacattca cagagttaaa | |
| 3001 | tgaagagtct cgaggttcta caatctctct gatgagaaca gattgtctat aaaatcccac | |
| 3061 | agtccttggg aagttgggga ccacatacac tgttgggatg tacattgata caacctttat | |
| 3121 | gatggcaatt tgacaatatt tattaaaata aaaaatggtt attcccttca tatcagtttc | |
| 3181 | tagaaggatt tctaagaatg tatcctatag aaacaccttc acaagtttat aagggcttat | |
| 3241 | ggaaaaaggt gttcatccca ggattgttta taatcatgaa aaatgtggcc aggtgcagtg | |
| 3301 | gctcactctt gtaatcccag cactatggga ggccaaggtg ggtgacccac gaggtcaaga | |
| 3361 | gatggagacc atcctggcca acatggtgaa accctgtctc tactaaaaat acaaaaatta | |
| 3421 | gctgggcgtg atggtgcacg cctgtagtcc cagctacttg ggaggctgag gcaggagaat | |
| 3481 | cgcttgaacc cgggaggtgg cagttgcagt gagctgagat cgagccactg cactccagcc | |
| 3541 | tggtgacaga gcgagactcc atctcaaaaa aaagaaaaaa aaaaaagaaa aaaa | |

The method can further include determining the genotype of the individual with respect to other Mgat3 or TLR alleles. Single nucleotide polymorphisms for MGAT3 and TLRs are shown in Table 1 below.

TABLE 1
Single nucleotide polymorphisms for MGAT3 and TLRs

| | | | | | | refer- | | | | | |
|---|---|---|---|---|---|---|---|---|---|---|---|
| | | mRNA | | | | ence | reference | alternate | alternate | codon | a.a. |
| Gene | chrom. | position | SNP ID | Heterozyg | Function | allele | a.a. | allele | a.a. | position | position |
| MGAT3 | 22 | 970 | rs5995741 | N.D. | nonsynonymous | C | Ala [A] | A | Asp [D] | 2 | 242 |
| | | 1287 | rs9611185 | N.D. | nonsynonymous | G | Gly [G] | A | Ser [S] | 1 | 348 |
| TLR3 | 4 | 655 | rs35140061 | 0.025 | nonsynonymous | C | Ala [A] | T | Val [V] | 2 | 185 |
| | | 952 | rs5743316 | 0.004 | nonsynonymous | A | Asn [N] | T | Ile [I] | 2 | 284 |
| | | 990 | rs35311343 | 0.025 | nonsynonymous | C | Leu [L] | G | Val [V] | 1 | 297 |
| | | 1020 | rs5743317 | 0.023 | nonsynonymous | T | Tyr [Y] | G | Asp [D] | 1 | 307 |
| | | 1335 | rs3775291 | 0.318 | nonsynonymous | C | Leu [L] | T | Phe [F] | 1 | 412 |
| | | 2310 | rs5743318 | 0.017 | nonsynonymous | T | Ser [S] | A | Thr [T] | 1 | 737 |
| TLR4 | 9 | 664 | rs16906079 | 0.031 | nonsynonymous | A | Thr [T] | G | Ala [A] | 1 | 175 |
| | | 704 | rs5030713 | N.D. | nonsynonymous | A | Gln [Q] | G | Arg [R] | 2 | 188 |
| | | 878 | rs5030714 | N.D. | nonsynonymous | G | Cys [C] | C | Ser [S] | 2 | 246 |
| | | 1037 | rs4986790 | 0.086 | nonsynonymous | A | Asp [D] | G | Gly [G] | 2 | 299 |
| | | 1059 | rs2770145 | N.D. | nonsynonymous | T | Cys [C] | G | Trp [W] | 3 | 306 |
| | | 1070 | rs2770144 | N.D. | nonsynonymous | T | Val [V] | G | Gly [G] | 2 | 310 |
| | | 1127 | rs5030715 | N.D. | nonsynonymous | A | Asn [N] | G | Ser [S] | 2 | 329 |
| | | 1166 | rs5031050 | N.D. | nonsynonymous | T | Phe [F] | A | Tyr [Y] | 2 | 342 |
| | | 1296 | rs11536884 | 0.005 | nonsynonymous | G | Leu [L] | T | Phe [F] | 3 | 385 |
| | | 1337 | rs4986791 | 0.042 | nonsynonymous | C | Thr [T] | T | Ile [I] | 2 | 399 |
| | | 1340 | rs4987233 | 0.01 | nonsynonymous | G | Ser [S] | A | Asn [N] | 2 | 400 |
| | | 1470 | rs5030716 | 0.021 | synonymous | C | Phe [F] | T | Phe [F] | 3 | 443 |
| | | | | | nonsynonymous | | | A | Leu [L] | 3 | 443 |
| | | 1561 | rs5030718 | 0.046 | nonsynonymous | G | Glu [E] | A | Lys [K] | 1 | 474 |
| | | 1671 | rs5030719 | 0.016 | nonsynonymous | G | Gln [Q] | T | His [H] | 3 | 510 |
| | | 1737 | rs34953464 | 0.026 | nonsynonymous | C | Phe [F] | A | Leu [L] | 3 | 532 |
| | | 1920 | rs5030720 | N.D. | nonsynonymous | G | Trp [W] | A | [Ter[*]] | 3 | 593 |
| | | 2222 | rs5030722 | N.D. | nonsynonymous | A | Lys [K] | G | Arg [R] | 2 | 694 |
| | | 2429 | rs5030723 | N.D. | nonsynonymous | G | Arg [R] | A | His [H] | 2 | 763 |
| | | 2641 | rs5030724 | N.D. | nonsynonymous | C | Gln [Q] | A | Lys [K] | 1 | 834 |
| TLR5 | 1 | 3178 | rs5744177 | 0.021 | nonsynonymous | A | Asp [D] | G | Gly [G] | 2 | 846 |
| | | 3105 | rs7512943 | N.D. | nonsynonymous | T | Phe [F] | C | Leu [L] | 1 | 822 |
| | | 2722 | rs5744176 | 0.021 | nonsynonymous | A | Asp [D] | G | Gly [G] | 2 | 694 |
| | | 2571 | rs5744175 | 0.021 | nonsynonymous | A | Ile [I] | T | Phe [F] | 1 | 644 |
| | | 2487 | rs5744174 | 0.384 | nonsynonymous | T | Phe [F] | C | Leu [L] | 1 | 616 |
| | | 2434 | rs5744173 | 0.021 | nonsynonymous | A | Asn [N] | C | Thr [T] | 2 | 598 |
| | | 2416 | rs2072493 | 0.25 | nonsynonymous | A | Asn [N] | G | Ser [S] | 2 | 592 |
| | | 2100 | rs5744171 | 0.021 | nonsynonymous | C | Leu [L] | A | Ile [I] | 1 | 487 |
| | | 1815 | rs5744168 | 0.12 | nonsynonymous | C | Arg [R] | T | [Ter[*]] | 1 | 392 |
| | | 1242 | rs4140966 | N.D. | nonsynonymous | T | Phe [F] | C | Leu [L] | 1 | 201 |
| | | 1069 | rs5744167 | 0.021 | nonsynonymous | A | Asn [N] | C | Thr [T] | 2 | 143 |
| | | 975 | rs5744166 | 0.042 | nonsynonymous | C | Pro [P] | G | Ala [A] | 1 | 112 |
| | | 886 | rs764535 | 0.071 | nonsynonymous | C | Thr [T] | T | Ile [I] | 2 | 82 |
| TLR7 | X | 171 | rs179008 | 0.215 | nonsynonymous | A | Gln [Q] | T | Leu [L] | 2 | 11 |
| | | 1482 | rs5743781 | 0.01 | nonsynonymous | C | Ala [A] | T | Val [V] | 2 | 448 |
| | | 1865 | rs34501186 | N.D. | nonsynonymous | A | Asn [N] | G | Asp [D] | 1 | 576 |
| | | 1878 | rs35160120 | N.D. | nonsynonymous | T | Phe [F] | C | Ser [S] | 2 | 580 |
| | | 1936 | rs36076482 | N.D. | nonsynonymous | G | Gln [Q] | T | His [H] | 3 | 599 |
| | | 1968 | rs36110053 | N.D. | nonsynonymous | C | Ser [S] | G | Cys [C] | 2 | 610 |
| | | 1997 | rs34729893 | N.D. | nonsynonymous | T | Ser [S] | A | Thr [T] | 1 | 620 |
| | | 2019 | rs34014664 | N.D. | nonsynonymous | G | Arg [R] | T | Ile [I] | 2 | 627 |
| | | 2040 | rs34557368 | N.D. | nonsynonymous | T | Leu [L] | G | [Ter[*]] | 2 | 634 |
| | | 2046 | rs35337229 | N.D. | nonsynonymous | G | Arg [R] | C | Thr [T] | 2 | 636 |
| TLR8 | X | 233 | rs5744077 | 0.076 | nonsynonymous | A | Met [M] | G | Val [V] | 1 | 28 |
| | | 2349 | rs5744082 | 0.004 | nonsynonymous | G | Arg [R] | A | Gln [Q] | 2 | 733 |
| TLR9 | 3 | 3278 | rs5743846 | 0.005 | nonsynonymous | G | Ala [A] | A | Thr [T] | 1 | 882 |
| | | 3222 | rs5743845 | 0.008 | nonsynonymous | G | Arg [R] | A | Gln [Q] | 2 | 863 |
| | | 2519 | rs34399053 | 0.028 | nonsynonymous | G | Gly [G] | A | Ser [S] | 1 | 629 |
| | | 2271 | rs17846009 | N.D. | nonsynonymous | G | Arg [R] | A | Gln [Q] | 2 | 546 |
| | | 1834 | rs41308230 | N.D. | nonsynonymous | G | Met [M] | T | Ile [I] | 3 | 400 |
| | | 930 | rs5743844 | 0.021 | nonsynonymous | C | Pro [P] | T | Leu [L] | 2 | 99 |
| | | 871 | rs5743843 | 0.005 | nonsynonymous | T | His [H] | G | Gln [Q] | 3 | 79 |
| | | 647 | rs5743842 | 0.024 | nonsynonymous | C | Arg [R] | T | Cys [C] | 1 | 5 |
| TLR10 | 4 | 2506 | rs4129008 | 0.021 | nonsynonymous | G | Arg [R] | A | Gln [Q] | 2 | 799 |
| | | | | | nonsynonymous | | | C | Pro [P] | | 799 |
| | | | | | nonsynonymous | | | T | Leu [L] | | 799 |
| | | 2433 | rs4129009 | 0.173 | nonsynonymous | A | Ile [I] | C | Leu [L] | 1 | 775 |
| | | | | | nonsynonymous | | | G | Val [V] | | 775 |
| | | | | | nonsynonymous | | | T | Phe [F] | | 775 |
| | | 2317 | rs11466660 | 0.006 | nonsynonymous | A | Tyr [Y] | G | Cys [C] | 2 | 736 |
| | | 1683 | rs11466658 | 0.024 | nonsynonymous | C | Arg [R] | T | Trp [W] | 1 | 525 |
| | | 1528 | rs11466657 | 0.044 | nonsynonymous | T | Ile [I] | C | Thr [T] | 2 | 473 |
| | | 1515 | rs11466656 | 0.012 | nonsynonymous | C | Arg [R] | G | Gly [G] | 1 | 469 |
| | | 1252 | rs11466655 | 0.142 | nonsynonymous | G | Gly [G] | A | Asp [D] | 2 | 381 |

In some embodiments, the determination is carried out by analyzing DNA according to well known methods, which include, for example, direct DNA sequencing of the wild-type Mgat3 or TLRs gene, allele specific amplification using the polymerase chain reaction (PCR) enabling detection of either wild-type or variant Mgat3 or TLR sequences, or by indirect detection of the wild-type or variant Mgat3 or TLR genes by various molecular biology methods including, e.g., PCR-single stranded conformation polymorphism (SSCP)-method or denaturing gradient gel electrophoresis (DGGE). Determination of the wild-type or variant Mgat3 or TLR genes can also be done by using the restriction fragment length polymorphism (RFLP)-method, which is particularly suitable for genotyping large number of samples. As used herein, “wild-type Mgat3 or TLR genes” refers to an allele of the Mgat3 or TLR genes that (a) encodes a gene product that performs the normal function of Mgat3 or TLRs and (b) does not contain Mgat3 or TLRs mutations.

The determination can also be carried out at the level of RNA by analyzing RNA expressed in the sample using various methods. Allele specific probes can be designed for hybridization. Hybridization can be done using, e.g., Northern blot, RNase protection assay, or in situ hybridization methods. RNA derived forms of the wild-type or variant Mgat3 or TLR genes can also be analyzed by converting tissue RNA first to cDNA and thereafter amplifying cDNA by an allele specific PCR-method and carrying out the analysis as for genomic DNA as mentioned above.

Particularly suitable methods for analyzing the nucleic acids include hybridization between the nucleic acid sample and an Mgat3 or TLR nucleic acid probe or primer specific for the wild-type or variant Mgat3 or TLR alleles. Accordingly, nucleic acid molecules particularly useful in accordance with the methods provided herein are oligonucleotides capable of hybridizing, under stringent hybridization conditions, with complementary regions of the Mgat3 or TLRs gene that include the site associated with any Mgat3 or TLR mutation.

A nucleic acid can be DNA or RNA, and single- or double-stranded. Oligonucleotides can be naturally occurring or synthetic, but are typically prepared by synthetic means. Oligonucleotides provided herein include segments of DNA, or their complements, corresponding to the human Mgat3 or TLR genes and including the nucleotide at position of key codons (corresponding to nucleotide positions as shown in SEQ ID NOs: 1-8), and/or a base adjacent thereto, of either the variant or wild-type allele. The segments are usually between 5 and 100 contiguous bases, and often range from 5, 10, 12, 15, 20, or 25 nucleotides to 10, 15, 30, 25, 20, 50 or 100 nucleotides. Nucleic acids between 5-10, 5-20, 10-20, 12-30, 15-30, 10-50, 20-50, or 20-100 bases are common.

Oligonucleotides provided herein can be RNA, DNA, or derivatives of either. The minimum size of such oligonucleotides is the size required for formation of a stable hybrid between the oligonucleotide and a complementary sequence on a nucleic acid molecule corresponding to the human Mgat3 or TLRs genes. Provided are oligonucleotides that can be used as, for example, probes to identify nucleic acid molecules or primers to produce nucleic acid molecules. Also provided are oligonucleotides that can be used as primers to amplify DNA.

In some embodiments, the oligonucleotide probes or primers include single base change of a Mgat3 or TLR polymorphism (positions of key codons) or the wild-type nucleotide that is located at the same position. The single base change or corresponding wild-type nucleotide can occur within any position of the oligonucleotide. Preferably the nucleotide of interest occupies a central position of a probe. In certain embodiments, the nucleotide of interest occupies a 3′ position of a primer.

Polymorphisms are detected in a target nucleic acid from an individual being analyzed. For assay of genomic DNA, virtually any biological sample (other than pure red blood cells) is suitable. For example, convenient tissue samples include whole blood, blood cells, semen, saliva, tears, urine, fecal material, sweat, buccal epithelium, skin and hair. For assay of cDNA or mRNA, the tissue sample must be obtained from an organ in which the target nucleic acid is expressed.

Methods described below require amplification of DNA from target samples. This can be accomplished by, e.g., PCR. See generally, e.g., PCR Technology: Principles and Applications for DNA Amplification (H. A. Erlich ed., Freeman Press, NY, N.Y., 1992); PCR Protocols: A Guide to Methods and Applications (Innis et al. eds., Academic Press, San Diego, Calif., 1990); Mattila et al., Nucleic Acids Res. 19, 4967 (1991); Eckert et al., PCR Methods and Applications 1, 17 (1991); PCR (McPherson et al. eds., IRL Press, Oxford); and U.S. Pat. No. 4,683,202.

Other suitable amplification methods include the ligase chain reaction (LCR) (see, e.g., Wu and Wallace, Genomics 4.560 (1989), Landegren et al., Science 241, 1077 (1988)), transcription amplification (see, e.g., Kwoh et al., Proc. Natl. Acad. Sci. USA 86, 1173 (1989)), self-sustained sequence replication (see, e.g., Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The identity of the base occupying a polymorphic site at key codon of the Mgat3 or TLR genes (Table 1) can be determined in an individual by several methods, which are described as follows.

Single Base Extension Methods

Single base extension methods are described by, e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to, but does not span a site of, potential variation in the target sequence. That is, the primer comprises a subsequence from the complement of a target polynucleotide terminating at the base that is immediately adjacent and 5′ to a polymorphic site. The term primer refers to a single-stranded oligonucleotide capable of acting as a point of initiation of template-directed DNA synthesis under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer but typically ranges from 15 to 40 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′ downstream primer that hybridizes with the complement of the 3′ end of the sequence to be amplified.

The hybridization is performed in the presence of one or more labeled nucleotides complementary to base(s) that may occupy the site of potential variation. For example, for biallelic polymorphisms, two differentially labeled nucleotides can be used. For tetra allelic polymorphisms, four differentially-labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation if the target sequence is present. Extension incorporates a labeled nucleotide thereby generating a labeled extended primer. If multiple differentially-labeled nucleotides are used and the target is heterozygous then multiple differentially-labeled extended primers can be obtained. Extended primers are detected providing an indication of which base(s) occupy the site of variation in the target polynucleotide.

Allele-Specific Probes

The design and use of allele-specific probes for analyzing polymorphisms is described by, e.g., Saiki et al., Nature 324, 163-166 (1986); Dattagupta, EP 235,726; Saiki, WO89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent such that there is a significant difference in hybridization intensity between alleles, and preferably an essentially binary response, whereby a probe hybridizes to only one of the alleles. Hybridizations are usually performed under stringent conditions that allow for specific binding between an oligonucleotide and a target DNA containing one the polymorphic site. Stringent conditions are defined as any suitable buffer concentrations and temperatures that allow specific hybridization of the oligonucleotide to highly homologous sequences spanning the Mgat3 or TLRs wild type or polymorphic site and any washing conditions that remove non-specific binding of the oligonucleotide. For example, conditions of 5×SSPE (750 mM NaCl, 50 mM Na Phosphate, 5 mM EDTA, pH 7.4) and a temperature of 23° C. are suitable for allele-specific probe hybridizations. The washing conditions usually range from room temperature to 60° C. Some probes are designed to hybridize to a segment of target DNA such that the polymorphic site aligns with a central position (e.g., in a 15 mer at the 7 position; in a 16 mer, at either the 8 or 9 position) of the probe. This probe design achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. The polymorphisms can also be identified by hybridization to nucleic acid arrays, some examples of which are described by WO 95/11995.

Allele-Specific Amplification Methods

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer that hybridizes at a distal site. Amplification proceeds from the two primers leading to a detectable product signifying that the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO93/22456. In other methods, a double-base mismatch is used in which the first mismatch is included in the 3′-most position of the oligonucleotide aligned with the polymorphism and a second mismatch is positioned at the immediately adjacent base (the pen-ultimate 3′position). This double mismatch further prevents amplification in instances in which there is no match between the 3′position of the primer and the polymorphism.

Direct-Sequencing

The direct analysis of the sequence of polymorphisms provided herein can be accomplished using either the dideoxy-chain termination method or the Maxam Gilbert method (see Sambrook et al., Molecular Cloning, A Laboratory Manual (2nd ed., CSHP, New York 1989); Zyskind et al., Recombinant DNA Laboratory Manual (Acad. Press, 1988)).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification (W. H. Freeman and Co, New York, 1992), Chapter 7.

Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. USA 86, 2766-2770 (1989). Amplified PCR products can be generated as described above, and heated or otherwise denatured, to form single stranded amplification products.

Single-stranded nucleic acids may refold or form secondary structures that are partially dependent upon the base sequence. The different electrophoretic mobilities of singlestranded amplification products can be related to base-sequence differences between alleles of target sequences.

Once the presence or absence of Mgat3 or TLR wild type or variant allele is determined for an individual, this information can be used in different ways. For example, as set forth above, a determination that the Mgat3 or TLR gene or enzyme is present is indicative of the susceptibility to disease or the efficacy of the drug for the treatment of a CNS disorder (e.g., AD or other neurodegenerative). Thus, the information can be used to help determine an appropriate diagnostic or treatment regimen, respectively, for an individual suffering from the disorder.

Determination of the presence or absence of the Mgat3 or TLR wild type or variant alleles is also useful for conducting clinical trials of drug candidates for CNS disorders. Such trials may be performed on treated or control populations having similar or identical polymorphic profiles at a defined collection of polymorphic sites. Use of genetically matched populations eliminates or reduces variation in treatment outcome due to genetic factors, leading to a more accurate assessment of the efficacy of a potential drug.

Furthermore, the determination of the presence or absence of the Mgat3 or TLR genes or a variant allele may be used after the completion of a clinical trial to elucidate differences in response to a given treatment. For example, the information may be used to stratify the enrolled patients into disease sub-types or classes. It may further be possible to use the methods described herein to identify subsets of patients with similar polymorphic profiles who have unusual (high or low) response to treatment or who do not respond at all (non-responders). In this way, information about the underlying genetic factors influencing response to treatment can be used in many aspects of the development of treatments (these range from the identification of new targets, through the design of new trials to product labeling and patient targeting). Additionally, the methods may be used to identify the genetic factors involved in adverse response to treatment (adverse events). For example, patients who show an adverse response may have a higher incidence of the absence of the Mgat3 or TLR allele than observed in the general population. This would allow the early identification and exclusion of such individuals from treatment. It would also provide information that might be used to understand the biological causes of adverse events and to modify the treatment to avoid such outcomes.

In another aspect, provided are methods for screening for Mgat3 or TLRs upregulation activity using the variant and/or wild-type Mgat3 or TLRs protein. These methods can provide information as to how to modify a drug candidate to make a more efficacious and/or safer drug for the treatment of a CNS disorder such as, e.g., AD.

In another aspect, provided is a method to remove blood cells from an AD patient, isolate and treat white or other blood cells with an agent that increases Mgat3 and/or TLR activity. After removal of the agent, the cells are returned to the AD patient for treatment of AD or other CNS diseases.

In certain embodiments, a predetermined therapeutic agent (e.g., curcumin) for the treatment of a CNS disorder is derivatized to create one or more analog candidate agents. The agent will typically retain one or more moieties associated with therapeutic efficacy, while incorporating one or more moieties that are or known or predicted to be a potential inducer moiety for Mgat3 or TLRs. Mgat3 or TLRs inducer moieties are not generally known but can include, for example, chemical centers such as, e.g., a chemical center analogous to that contained curcumin.

Methods of chemical modification suitable for use in accordance with the methods provided herein are generally known in the art. For example, an Mgat3 or TLRs inducer moiety (e.g., a curcumin group) can be linked to the predetermined therapeutic agent, or be an inducer itself.

The derivatized agent is tested to determine if the agent is an inducer for the Mgat3 or TLR protein. Greater levels of Mgat3 enzyme or TLR activity in the presence of the derivatized agent relative to the underivatized, predetermined therapeutic agent is generally indicative of greater efficacy and/or lower toxicity of the derivatized agent relative to the underivatized therapeutic agent. In certain embodiments, a library of derivatized agents is screened to identify one or more candidate agents that are inducer for Mgat3 or TLRs. Mgat3 or TLRs proteins suitable for use in accordance with these methods include, e.g., wild-type and variant Mgat3 or TLRs.

In one embodiment, a method for predicting the efficacy of a candidate agent for the treatment of a CNS disorder is provided which includes: (1) contacting a wild type sample of an Mgat3 or TLR protein with the candidate agent; (2) contacting a second AD sample of an Mgat3 or TLR protein with a predetermined therapeutic agent; where the contacting of each of the first and second samples is under conditions suitable for supporting Mgat3 enzyme or TLR activity; (3) determining for each of the first and second samples the level of Mgat3 enzyme or TLR activity; and (4) comparing the level of Mgat3 enzyme or TLR activity in the first sample with the level of Mgat3 enzyme or TLR activity in the second sample. A greater level of Mgat3 enzyme or TLR activity in the second sample relative to the first sample is indicative of efficacy of the candidate agent for treatment of the disorder. In certain embodiments, the predetermined therapeutic agent is an anti-AD drug such as, e.g., curcumin or some other immune modulator. Particularly suitable are candidate agents having a curcumin center analogous to the center of curcumin.

The Mgat3 or TLR protein sample can include, e.g., a sample comprising a recombinant form of the protein in a cellular or a cell-free preparation. Methods for producing and isolating catalytically active, recombinant human Mgat3 or TLR protein are known in the art. (See, e.g., Bhattacharyya et al., J. Biol. Chem. 277:26300-26309 (2002).

Mgat3 or TLR protein suitable for use in accordance with the present methods can also be obtained from tissues or cells that express the Mgat3 or TLR protein endogenously. For example, tissues or cells expressing Mgat3 or TLR protein may be use to prepare enzyme for use in Mgat3 or TLR enzyme activity assays. Kidney or brain is a particularly suitable source for Mgat3 or TLR protein. Kidney or brain samples suitable for use in the preparation of enzyme can be obtained from banks of cryopreserved human or mouse tissue. Methods for preparing human or mouse kidney or brain containing viable Mgat3 or TLR protein, and as well as method for using enzyme assays in Mgat3 or TLR activity assays, are known. (See, e.g., Bhattacharyya et al., J. Biol. Chem. 277:26300-26309 (2002)). In certain embodiments, tissues or cells used for preparation of Mgat3 or TLR protein are homozygous for either variant or wild-type Mgat3 or TLR. In other embodiments, a protein sample containing variant Mgat3 or TLR is derived from tissue or cells heterozygous for a variant allele.

In certain embodiments, the sample comprises cells, cultured in vitro, expressing Mgat3 or TLR. The cells can express either recombinant or endogenous Mgat3 or TLR protein. Particularly suitable cells for endogenous expression of Mgat3 or TLR include human kidney cells or transfected CHO cells. Cells expressing an endogenous variant Mgat3 or TLR allele can be either homozygous or heterozygous. With respect to recombinant cells, methods for cloning genes encoding the Mgat3 or TLR protein, production of recombinant expression vectors, transfection of cells, and subsequent expression of the encoded protein are known in the art. (See generally, e.g., Sambrook and Russell, Molecular Cloning, A Laboratory Manual (3rd ed. 2001); Ausubel et al. (eds.), Current Protocols in Molecular Biology (1994); Sambrook et al., Molecular Cloning, A Laboratory Manual (2nd ed. 1989).) Methods for determining Mgat3 (or TLR) activity in cultured cells are also generally known in the art. (See, e.g., Bhattacharyya et al., J. Biol. Chem. 277:26300-26309 (2002)).

Suitable methods for determining the level of Mgat3 (or TLR) enzyme activity typically include, for example, detection of N-glycosylation associated with Mgat3 enzyme activity or binding to TLR. For Mgat3, a particularly suitable assay is the detection of an N-glycosylation of a peptide or a protein (See, e.g., Bhaumik et al., Cancer Res. 58, 2881-2887). For example, the method can include detection of a N-glycosylated peptides or proteins.

Methods of sample preparation and product identification, including identification of N-glycosylated products, are well-known in the art and include, for example, the use of HPLC methods (e.g., reverse HPLC-tandem mass spectrometry (HPLC-MS/MS) or TLC methods). (See, e.g., Bhaumik et al., Cancer Res. 58, 2881-2887).

Ex Vivo Therapy of Alzheimer Disease

In another embodiment, provided is a method for ex vivo therapy for patients with AD. This method comprising the steps of obtaining a blood sample from an AD patient, contacting the blood sample with the compounds provided herein and injecting the treated blood sample back into the AD patient.

Pharmaceutical Compositions

Provided herein are pharmaceutical compositions comprising one or more compounds of Formula I as active ingredients or a pharmaceutically acceptable salt, solvate, or prodrug thereof, in a pharmaceutically acceptable vehicle, carrier, diluent, or excipient, or a mixture thereof.

Provided herein are pharmaceutical compositions in modified release dosage forms, which comprise one or more compounds of Formula I or a pharmaceutically acceptable salt, solvate, or prodrug thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further provided herein are pharmaceutical compositions in enteric coated dosage forms, which comprise one or more compounds of Formula I or a pharmaceutically acceptable salt, solvate, or prodrug thereof; and one or more release controlling excipients for use in an enteric coated dosage form. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours.

In one embodiment, the pharmaceutical compositions comprise one or more compounds of Formula I or a pharmaceutically acceptable salt, solvate, or prodrug thereof; and one or more release controlling and non-release controlling excipients, such as those excipients suitable for a disruptable semi-permeable membrane and as swellable substances.

Provided herein are pharmaceutical compositions that comprise about 0.1 to about 100 mg, about 0.5 to about 75 mg, about 1.0 to about 50 mg, about 2.5 to about 25.0 mg, about 5.0 to about 15 mg, about 0.1 mg, about 0.5 mg, about 1 mg, about 5 mg or about 10 mg, of one or more compounds of Formula I as a sterile solution for injection per day. The pharmaceutical compositions further comprise about 0.1% to about 2% sodium chloride, about 0.1% to about 2% ammonium acetate, about 0.001% to about 0.1% edetate disodium, about 0.1% to about 2% benzyl alcohol, with a pH of about 6 to about 8.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampouls, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary depending on a condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Routes of Administration

Depending on the condition, disorder, or disease, to be treated and the subject's condition, a compound provided herein may be administered by oral, parenteral (e.g., intramuscular, intraperitoneal, intravenous, ICV, intracisternal injection or infusion, subcutaneous injection, or implant), inhalation, nasal, vaginal, rectal, or sublingual routes of administration, and may be formulated, alone or together, in suitable dosage unit with pharmaceutically acceptable carriers, adjuvants and vehicles appropriate for each route of administration.

Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcellulose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogens of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

Controlled-Release Dosage Forms

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiment, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

Dosing

In certain embodiments, provided compounds are administered once daily in a single or divided dose in the amount of about 0.1 to about 100 mg/kg per day for parenteral administration, where kg refers to a subject's body weight.

In certain embodiments, provided compounds are administered once daily in a single or divided dose in the amount of about 0.5 to about 75 mg/kg per day.

In certain embodiments, provided compounds are administered once daily in a single or divided dose in the amount of about 1.0 to about 50 mg/kg per day.

In certain embodiments, provided compounds are administered once daily in a single or divided dose in the amount of about 2.5 to about 25.0 mg per day.

In certain embodiments, provided compounds are administered once daily in a single or divided dose in the amount of about 5.0 to about 15 mg per day.

In certain embodiments, provided compounds are administered once daily in a single or divided dose in the amount of about 0.1 mg, about 0.5 mg, about 1 mg, about 5 mg or about 10 mg of one or more compounds of Formula I for parenteral administration per day.

The following non-limiting examples are provided below.

### EXAMPLES

### Example 1

Molecular cloning of human Mgat3 or TLR. The human Mgat3 or TLR gene is cloned using an RT-PCR method. Genomic DNA is prepared from human kidney or human brain provided by a commercial source. Total RNA is prepared from kidney of human tissue using the Trizol reagent via a standard protocol. Superscript pre-amplification system is used to synthesize the first strand cDNA from total RNA using oligo dT primers. The primers for RT-PCR were designed based on wild type human Mgat3 or TLR sequences. Human Mgat3 or TLR genes are amplified from cDNA with Platinum Taq DNA polymerase high fidelity. A human Mgat3 or TLR PCR fragment of the appropriate full length is obtained. The appropriate fragments for all the exons is obtained for the human Mgat3 or TLR DNA. PCR products are fully sequenced in both directions to determine the complete cDNA sequence of human Mgat3 or TLR.

Sub-cloning of Mgat3 or TLR into an expression vector. The full length Mgat3 or TLR is sub-cloned into an expression vector for expression of the Mgat3 or TLR protein in CHO or LEC10 cells.

Expression of recombinant Mgat3 or TLR in CHO cells. The cDNA encoding the Mgat3 or TLR protein is expressed in CHO cells after selection by G418. Protein expression was followed by SDS-PAGE and Western blots analysis.

### Example 2

Expression of recombinant Mgat3 or TLR. The human Mgat3 or TLR cDNA is cloned and expressed in CHO cells. Western blot analysis shows that the recombinant Mgat3 or TLR was expressed and recognized by an anti-human Mgat3 or TLR polyclonal antibody. Lineweaver Burk studies are done with prototypical peptides and protein substrates of Mgat3 or binding studies done with TLR. The catalytic efficiency of Mgat3 is ascertained with peptide or protein substrates. The activity of TLR is measured with binding studies or functional activity measurements.

Analysis of the cDNA sequence of Mgat3 or TLR. RT-PCR is used to clone the cDNA for Mgat3 or TLRs from human tissue and to obtain the genomic DNA for human Mgat3 or TLRs. The longest open reading frame of Mgat3 or TLR encodes a polypeptide having sequence identity with human wild-type Mgat3 (GenBank Accession NM 002409) or TLR (GenBank Accession NM 003265 (TLR3), NM 138554 (TLR4), NM 003268 (TLR5), NM 016562 (TLR7, NM 138636 (TLR8), NM 017442 (TLR9), NM 030956 (TLR10)), respectively, at all amino acid positions.

### Example 3

Peripheral blood mononuclear cells (PBMC's) and macrophages. AD patients were recruited at the time of enrollment in a double-blind study of curcumin Complex 3 in progress at UCLA. The diagnostic criteria for AD satisfied the National Institute of Neurological and Communicative Disorders and the AD and Related Disorders Association criteria for probable Alzheimer's disease. Normal age-matched control subjects were recruited. PBMC's were isolated by the Ficoll Hypaque gradient technique from venous blood. To prepare macrophage slide cultures, 50,000 PBMC's were cultured in each well of an 8-chamber polystyrene vessel glass slide in Iscove's medium with 10% autologous serum until differentiated into adherent macrophages (7-14 days).

Phagocytosis assay and confocal microscopy. Macrophages were exposed to FITC-amyloid-beta (1-42) (2 μg/ml) overnight, fixed with 4% paraformaldehyde, permeabilized with 0.1% Triton X-100, blocked with 1% BSA in PBS and stained with Rab 5 or EEA1 antibodies by indirect immunofluorescence; lysosomes were stained intravitally by the Lyso-Tracker probe; macrophages were stained using anti-CD68; neurons were stained using anti-NeuN. The preparations were examined using fluorescence and confocal microscopy.

Clearance of Aβ in brain slices. Six micrometer sections from frozen brain frontal lobe tissues of AD patients were incubated in DMEM with 10% FBS with 200,000 PBMC's for 2 or 4 days, washed, fixed with 4% paraformaldehyde and stained by indirect immunofluorescence using antibodies to CD68 and Aβ (1-42) and appropriate secondary antibodies.

### Example 4

RNA and Microarray Probe Preparation and Hybridization. 10 million PBMC's of AD patients and controls were cultured overnight with and without Aβ (2 μg/ml). RNA was isolated by the RNeasy Mini kit technique. Total RNA (1 μg) from each sample and the reference (Universal Human Reference RNA) were used in probe preparations. Reverse transcription driven by an oligo (dT) primer bearing a T7 promoter using ArrayScript then underwent second strand synthesis and clean-up to become a template for in vitro transcription with T7 RNA Polymerase. MEGAscript® in vitro transcription was used to generate amplified RNA (aRNA). The antisense aRNA was then fluorescently labeled with Cy3 (reference) and Cy5 (sample). Sample and reference aRNAs were pooled, mixed with 1× hybridization buffer (50% formamide, 5×SSC, and 0.1% SDS), COT-1 DNA, and poly-dA to limit nonspecific binding, and heated to 95° C. for 2 minutes. This mixture was placed onto a microarray slide, and hybridized overnight at 42° C. The array was then washed at increasing stringencies, and scanned on a microarray scanner. Human oligonucleotide arrays were printed representing 24,650 genes. For analysis, two groups with two replicates each were created. Each dataset was normalized to the mean signal value for the set and ANOVA was performed. Genes with P<0.05 and a fold change of at least 3-fold were selected for further testing by qPCR.

RNA Isolation and qPCR. RNA was isolated as above from PBMC's (10 million) of each subject, which were cultured overnight with or without Aβ (2 μg/ml); and cDNA was synthesized using the iSCRIPT cDNA Synthesis Kit. The expression levels of the genes of interest were tested by qPCR on a real-time PCR detector and normalized to the levels of the housekeeping gene 36B4 with the following primers:

| | | |
|---|---|---|
| FN1, | 5′-ATGGGAGAAGTATGTGCATGGTG; | (SEQ ID NO: 9) |
| | 3′-CGGCCATAGCAGTAGCACTG; | (SEQ ID NO: 10) |
| MGAT3, | 5′-TTCGCCTTCCACATGCG; | (SEQ ID NO: 11) |
| | 3′-GTGCCCGGCTGCTTCC; | (SEQ ID NO: 12) |
| OAS3, | 5′-AGCCAGCATCGTACCCCTCT; | (SEQ ID NO: 13) |
| | 3′-TCTGAGACAGGTCCAAGGCC; | (SEQ ID NO: 14) |
| EF1AY, | 5′-TGCAGATGAAGCTAGAAGCCTG; | (SEQ ID NO: 15) |
| | 3′-GCATGTTCTGGAAGCTCGC; | (SEQ ID NO: 16) |
| | 5′-ATGCACACAAACATGGCACAG; | (SEQ ID NO: 17) |
| | 3′-AAATGCGTGGAATCGGACAT; | (SEQ ID NO: 18) |
| h36B4, | 5′-CCACGCTGCTGAACATGCT; | (SEQ ID NO: 19) |
| | 3′-TCGAACACCTGCTGGATGAC; | (SEQ ID NO: 20) |
| 36B4, | 5′-CCACGCTGCTGAACATGCT-3′; | (SEQ ID NO: 21) |
| | 5′-TCGAACACCTGCTGGATGAC-3′; | (SEQ ID NO: 22) |
| MGAT3, | 5′-CGTGGTGGACGCCTTTGT-3′; | (SEQ ID NO: 23) |
| | 5′-TCCCCATAAGCCGTGAAGTT-3′. | (SEQ ID NO: 24) |

SYBR Green reactions were carried out with the IQ SYBR Green mix. Reactions were run on a Continuous Fluorescence detector and analyzed. The relative quantities of the gene tested per sample were calculated against 36B4 using the ΔΔC(T) formula as previously described. The results are expressed as log [MGAT (or TLR) RNA (with Aβ)/MGAT (or TLR) RNA (without Aβ)] for each specimen. For evaluation of curcumins or curcumin analogs the above assay was used. Curcumins (0.1 uM) were added to PBMC's from AD patients cultured overnight with ±Aβ as above. After 2 hours of incubation with the curcumin or analog, RNA was isolated and cDNA was synthesized as above. The expression levels of Mgat3 or TLR transcription was quantified and normalized to the levels of housekeeping genes and compared to cells treated with curcumin or analogs with or without Aβ vs. untreated cells. The amount of Mgat3 (or TLR) RNA with Aβ and test agent/Mgat3 (or TLR) RNA withal alone)] for each cell preparation in the presence of test agent was determined. The ratio was used to determine the potency of the agent tested. Potent compounds had elevated ratios (>1.5) and were used to rank the relative activity of each test agent (Table 2).

DNA Samples. Genomic DNA was obtained from blood from the subjects described above. Genomic DNA was extracted from blood under standard conditions and individual exons and immediate flanking intronic regions were amplified from genomic DNA in the presence of specific primers as described previously.

Sequencing. Sequencing was done for both forward and reverse strands and analyzed with Sequencher software by procedures that resolve heterozygotes under reliable quality control conditions. The full length sequence for Mgat3 and TLR is shown above in SEQ ID NOs: 1-8.

### Example 5

Phagocytosis by macrophages of healthy and AD subjects. On the basis of studies with macrophages of 42 control subjects (“control macrophages”), ˜80% showed excellent or, rarely ˜10%, extremely efficient phagocytosis of soluble FITC-Aβ in 24 hours. In contrast, macrophages of 73 AD patients (“AD macrophages”) displayed either minimal surface uptake of FITC-Aβ (60%), no intracellular but strong surface uptake (25%), or extremely efficient phagocytosis (15%). When present, intracellular transport of Aβ was rapid in control macrophages but transport progressed slowly or not at all in macrophages from AD patients. One and two hr post-exposure of control macrophages, FITC-Aβ co-localized with the early endosomal marker Rab 5, whereas Rab5 staining and co-localization were minimal in AD macrophages. Co-localization with the transferrin receptor EEA1 was apparent in control macrophages but not in AD macrophages. Progression of the Aβ from the cell surface to lysosomes was not observed over a 72-hr period in AD macrophages, whereas in control macrophages FITC-Aβ became internalized at 1 hr post-exposure. FITC-Aβ co-localized with the lysosomal marker Lysotracker at 1, 48 and 72 h after explosure. In contrast, in AD macrophages, the Aβ bound to the cell surface and did not progress to lysosomes over a 72 h period, and the lysosomes were poorly expressed. Macrophages from both control and AD individuals showed efficient phagocytosis of fluorescently labeled E. coli and S. aureus. Scrambled Aβ (42-1) was not bound or internalized by control or AD macrophages. Tyrosine phosphorylation during phagocytosis was noted in control but not AD macrophages. Fucoidan treatment did not block uptake of Aβ.

### Example 6

Ability of monocytes to clear Aβ in the brain. Co-culture of freshly isolated monocytes with sections of AD frontal lobe to test the ability of monocytes to clear Aβ in the brain was done. One third of control monocytes became saturated with Aβ in 2 days and 100% in 4 days. In the same brain sections less than one quarter of AD monocytes became saturated with Aβ in 2 days; in 4 days, these monocytes (with and without internalized Aβ) showed fragmentation, blebbing and release of Aβ suggestive of apoptosis. Apoptosis of macrophages treated with Aβ was done with the SR-VAD-FMK polycaspase assay. Differentiated macrophages were treated with curcuminoids or analogs in the medium overnight and were then exposed to FITC-Aβ (1-42) to 2.5 μg/ml, incubated for 24 or 48 h and examined by fluorescence or confocal microscopy. Microarray testing showed down-regulation of Mgat3 in PBMC's of AD patients (in comparison to age-matched controls). Treatment of PBMC's of AD patients (in comparison to age-matched controls) with curcuminoids or analogs dramatically up-regulated the Mgat3 and TLR genes or changed the extent of phagocytosis (see below).

Curcuminoids reverse defective phagocytosis of amyloid-beta by macrophages of individual AD patients. To reverse the defect in phagocytosis, we treated macrophages with curcuminoids during overnight Aβ phagocytosis. Curcuminoid treatment was effective in macrophages of two AD patients to increase the uptake as shown by immunofluorescence microscopy but did not affect the uptake by control macrophages, which already had high uptake at baseline. Most importantly, the increase in uptake was through induction of intracellular phagocytosis, as shown by confocal microscopy. Macrophages were visualized using anti-CD68 or fluorescent phalloidin with a fluorescence microscope.

### Example 7

Transcriptional alterations in AD mononuclear cells during Aβ phagocytosis. To determine transcriptional alterations in AD mononuclear cells during Aβ phagocytosis, microarray analysis on the Operon platform of mRNA's isolated from mononuclear cells of 2 AD patients and 2 controls was done. Compared with control cells treated with Aβ, AD cells treated with Aβ, up-regulated (>3 fold) the transcription of 33 genes including β-1,4-mannosyl-glycoprotein 4-β-N-acetylglucosaminyltransferase (Mgat3) (327-fold in control macrophages (P<0.001), fibronectin (FN1) (10.1 fold), cholinergic receptor muscarinic 4 (9.3-fold), and 2′-5′-oligoadenylate synthetase 3 (OAS) (7.8-fold), and down-regulated (>3fold) the transcription of 8 genes. We confirmed this using qPCR (the transcriptional changes of Mgat3, OAS, FN1, and investigated the range of responses of Mgat3) in mononuclear cells of 14 patients and 8 controls. A majority of AD patients (71.5%) down-regulated Mgat3 RNA on Aβ stimulation (ratio 0.00001 to 1.0) but 4 AD patients up-regulated the expression of Mgat3. Control subjects up-regulated Mgat3 RNA on Aβ stimulation with the exception of two subjects >80 years old that down-regulated the response. Additional studies showed that Mgat siRNA transfection of control macrophages inhibited up-regulation of Mgat3 (by 99%) and uptake of FITC-Aβ per monocyte (86%). When both phagocytosis and Mgat3 transcription were tested simultaneously, AD patients showed lower scores on both. The product of the Mgat3 gene is N-acetylglucosaminyltransferase III (GlcNAc-TIII), which transfers the bisecting N-acetylglucosamine to the core mannose of complex N-glycans. GlcNAc-TIII regulates protein N-glycosylation and modulates cell interactions. Animals with truncated or inactive GlcNAc-TIII have neurological dysfunction. Thus, abnormal Mgat3 genes will predispose individuals to neurodegenerative disease and behavioral disorders including AD. The downstream effect of Mgat3 on phagocytosis may depend upon TLRs.

### Example 8

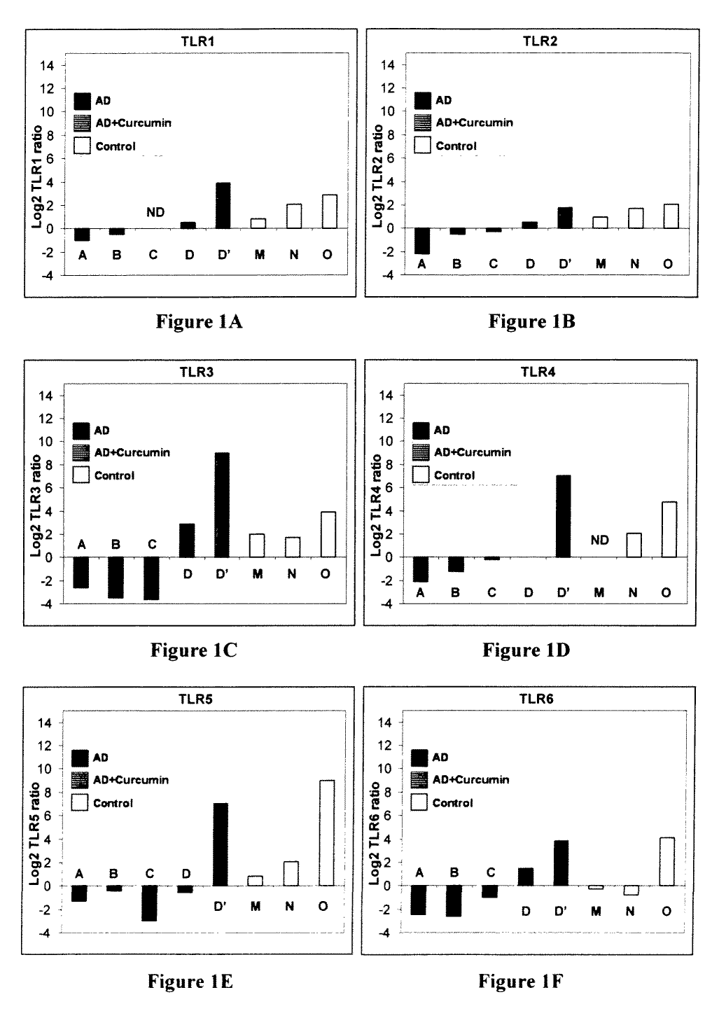

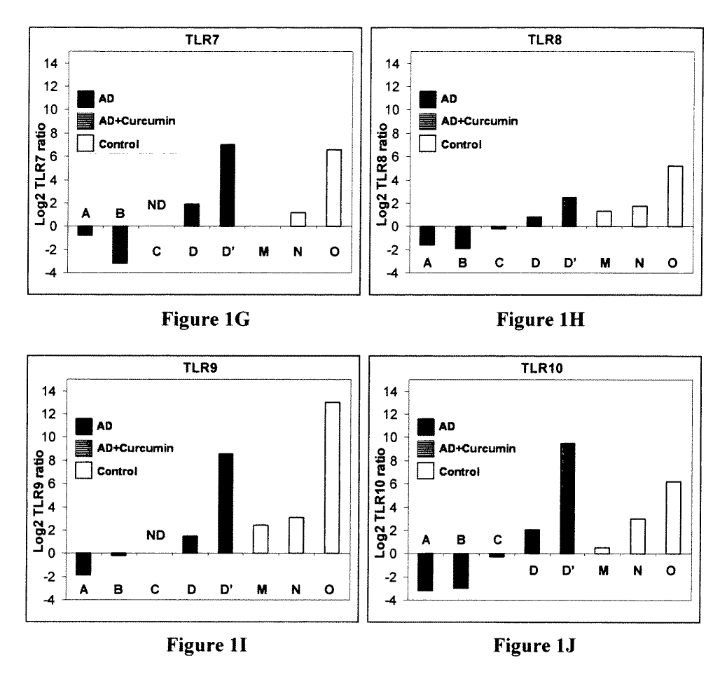

Down-regulation of TLRs in AD patients. We tested by qPCR TLR transcription in 18 AD patients and 9 control subjects and found that TLRs are significantly down-regulated in AD patients in the age-group 60-90 years of age. In the subgroup 81-90, this association is not present. Activation of TLR's results in many functional outcomes, including the enhancement of apoptosis, secretion of inflammatory cytokines, and direct antimicrobial activity. PBMC's from AD patients generally have down-regulated TLR, whereas control PBMC's had up-regulated TLR. Transcription of TLR1, TLR2, TLR3, TLR5, TLR8, and TLR10 upon Aβ stimulation is significantly down-regulated in AD compared to control mononuclear cells (FIG. 1). TLR, TLR4, TLR5, TLR7, TLR8, TLR9 and TLR10 showed the greatest difference between AD patients and controls. Repeat assays of TLR of control subjects showed up-regulation and those of AD patients showed down-regulation. The lower expression levels of TLR's on AD macrophages may be indicative of more global innate immune defects beyond Aβ phagocytosis.

### Example 9

Curcumins. Bisdesmethoxycurcuminoid is among the most potent immunoenhancing curcuminoid compounds identified, which also up-regulates MGAT3 and TLR transcription. Crude natural product derived materials (i.e., curcuminoids) enhance phagocytosis of Aβ by macrophages from AD patients in approximately 50% of the cases examined. By an iterative process that was bioassay-directed according to the FITC-Aβ uptake (IOD) to identify active fractions from curcuminoids, we isolated the most potent immunostimulatory component. The material was purified to near homogeneity and identified by LCMS as bisdesmethoxycurcumin on the basis of its molecular ion and fragmentation pattern. To verify the biological activity of this minor constituent, bisdesmethoxycurcumin was chemically synthesized and tested in the phagocytosis and transcription assays described above (see Example 4). Compared with curcumin, both the bisdesmethoxycurcumin material isolated by chromatography and the chemically synthesized bisdesmethoxycurcumin material optimally stimulated phagocytosis at 0.1 μM. To determine whether functional improvement would be accompanied by biochemical changes, we tested transcriptional up-regulation of MGA T3 and TLR's in PBMC's from AD patients and controls in the presence of Aβ with bisdesmethoxycurcumin (0.1 μM) in comparison to Aβ alone. Bisdemethoxycurcumin improved the transcription of MGAT3 and TLRs that were up-regulated in all four patients examined. Thus, curcumins (0.1 uM) were added to PBMC's from AD patients cultured overnight with Aβ as above. After 2 hours of incubation, RNA was isolated and cDNA was synthesized. The expression levels of Mgat3 or TLR transcription was quantified and normalized to the levels of housekeeping genes and compared to cells treated with curcumin or analogs without Aβ. The amount of Mgat3 (or TLR) RNA with test agent and Aβ)/Mgat3 (or TLR) RNA (with Aβ alone)] for each cell preparation in the presence of test agent was determined. The ratio was used to determine the potency of the agent tested. Potent compounds had elevated ratios (>1.5) and were used to rank the relative activity (Table 2). Bisdemethoxycurcumin treatment of PBMCs from an AD patient showed all 10 TLRs were up-regulated. Flow cytometry of PBMCs treated with bisdemethoxycurcumin from an AD patient showed increased expression of TLR2, TLR3 and TLR4 on monocytes.

Purification of Curcumins. One gram of curcumin placed in 75 mL of dichloromethane was filtered and the mother liquor evaporated and approximately 50 mg of the extract was placed on a silica gel PTLC plate, eluted with dichloromethane and gave four prominent UV vis-active components (R_{f} 0.27, 0.14, 0.08 and 0.06, respectively). The most active fraction in a bioassay-guided fractionation of curcumin led to the isolation of bisdemethoxycurcumin as the potent curcuminoid that enhanced the phagocytosis of Aβ by macrophages of AD patients. The active fraction was further separated with PTLC using methanol:dichloromethane (14:86, v:v). Three prominent fractions were visualized having R_{f} values of 0.69, 0.63 and 0.49 and were isolated, extracted and evaporated. Judged to be greater than 80% pure on the basis of TLC analysis, the three fractions were sent for bioassay-guided analysis. The fraction with an R_{f} value of 0.49 showed the greatest activity and it was investigated further by LCMS. Approximately 4.5 mg of the active fraction was analyzed on RPLCMS eluted with a gradient starting from acetonitrile:water (5:95, v:v) to acetonitrile:water (95:5, v:v) at a rate of 1.5 ml/min over five minutes with UV detection set at 220 nm. A prominent material eluted with a retention time of 2.17 min and was judged to be approximately 90% pure and showed a prominent ion of m/z 308. A larger ion at m/z 290 (arising from loss of water) was also observed. A subsequent electrospray mass spectrometry experiment also showed the anticipated m/z 309 and m/z 291 for the [M+1] ions. On the basis of the HPLC-mass spectrometry experiments, the isolated fraction showing the greatest pharmacological activity corresponded to the minor curcumin, bisdemethoxycurcumin. No detectable amounts of other curcurmins were observed present in this fraction on the basis of mass spectrometric analysis.

Synthesis of bisdemethoxycurcumin. Subsequent to the identification of bisdemethoxycurcumin (5-Hydroxy-1,7-bis-(4-hydroxy-phenyl)-hepta-1,4,6-trien-3-one) as the most active fraction in the bioassay-guided fractionation, it was independently synthesized and tested. It too showed considerable activity. Acetylacetone (2 ml, 19.5 mmol) and boric anhydride (1 g, 12.8 mmol) was stirred at RT under argon. 4-hydroxybenzaldehyde (9.52 g, 78.0 mmol) (or other benzaldehyde) was dissolved in dry ethylacetate (150 ml) tributyl borate (21 ml, 78.0 mmol) was added and the mixture was heated to 100° C., stirred for one hour and the boron complex from the first reaction was added to this mixture. The reaction mixture was stirred at 100° C. for one hour. The mixture was cooled to 85° C. and 1.9 mL butylamine (total 7.7 ml, 78 mmol) was added every 5 minutes. The mixture was stirred at 100° C. for 30 min, then cooled to 50° C. HCl, (0.4 N, 60 ml) was added and the mixture was stirred for another 30 min. The two layers were separated and the organic extract was washed with water and brine successively. The solution was dried over Na₂SO₄, filtered and concentrated to dryness. The crude product was chromatographed (2:1, hexane/EtOAc) to afford 0.98 g, 16% yield of the desired product as an orange powder. R_{f}=0.15, mp=199.9° C. ESI-MS: m/z 309 (MH)⁺, 331 (MNa)⁺, 307 (MH)⁻; ¹HNMR δ 7.50 (d, 2H, Ph-CH—), 7.29 (m, 4H, Ph), 6.62-6.78 (m, 4H, Ph), 6.37 (d, 2H, —CH—), 5.70 (s, 1H, —CO—CH—CO—).

Synthesis of Bisdemethoxycurcumin Analogs. The curcumin derivatives 5a-m were synthesized as outlined in Scheme 1 (Bull. Korean. Chem. Soc. 2004, 25, 1769-1774; Eur J. Med Chem, 1997, 32, 321-328.). Briefly, acetylacetone was treated with boric anhydride to give the boron complex 1. Condensation of the aldehydes 2a-m with the boron complex 1 in the presence of n-butylamine followed by acid dehydration afforded the curcumin derivatives 5a-m as described above. The compounds were fully characterized spectrally.

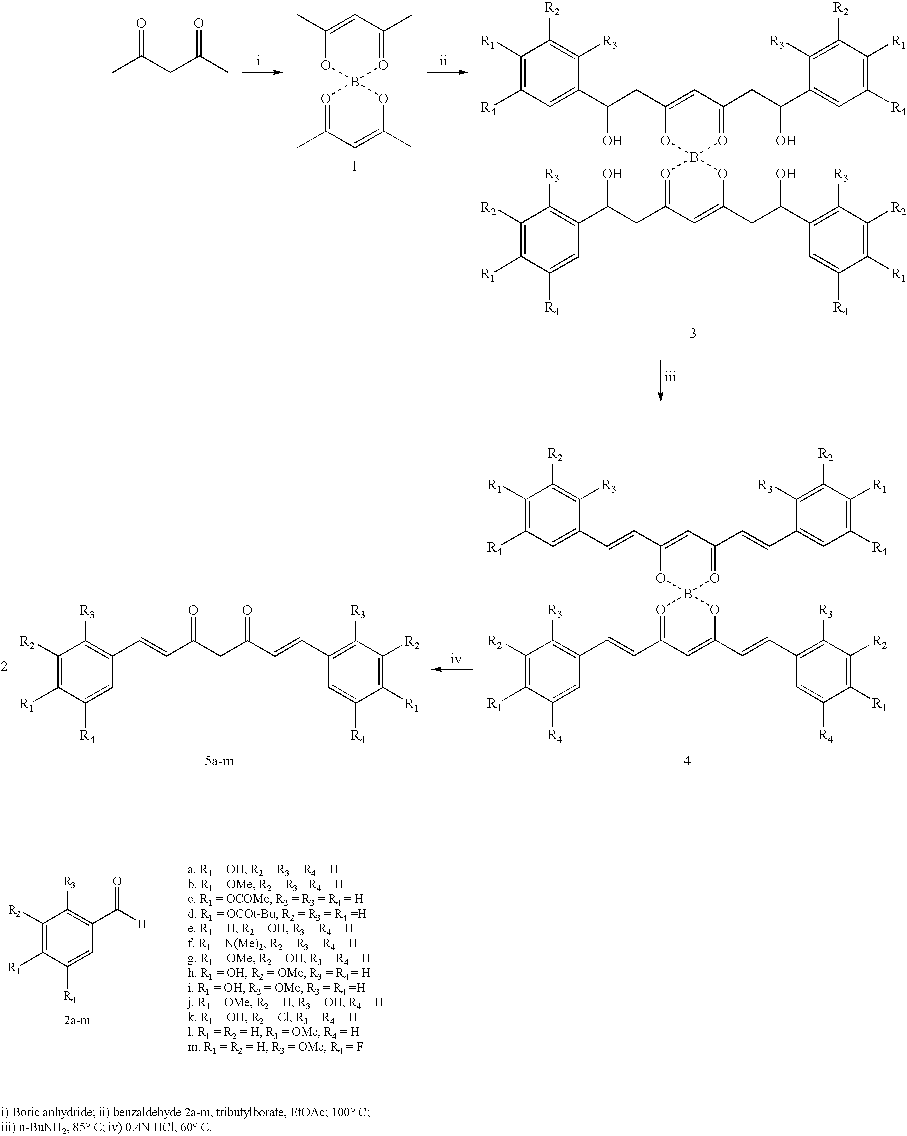

5-Hydroxy-1,7-bis-(4-methoxy-phenyl)-hepta-1,4,6-trien-3-one, 5b was prepared according to the general procedure described for compound 5a to give an orange powder. R_{f}=0.18; ESI-MS m/z 335 (MH⁻); ¹HNMR δ 7.62 (d, J=15.9 Hz, 2H, Ph-CH—), 7.53 (m, 4H, Ph), 6.93 (m, 4H, Ph), 6.51 (d, J=15.9 Hz, 2H, —CH—), 5.80 (s, 1H, —CO—CH—CO—)

Aceticacid 4-[7-(4-acetoxy-phenyl)-5-hydroxy-3-oxo-hepta-1,4,6-trienyl]-phenyl ester, 5c. Bisdemethoxycurcumin, 5a was treated with acetylchloride/TEA and gave a yellow powder. R_{f}=0.71; ¹HNMR δ 7.70 (d, J=18.0 Hz, 2H, Ph-CH—), 7.61 (m, 4H, Ph), 7.17 (m, 4H, Ph), 6.60 (d, J=18.0 Hz, 2H, —CH—CO—), 5.87 (s, 1H, —CH—), 2.35 (s, 6H, 2×CH₃).

2,2-Dimethyl-propionic acid 4-{7-[4-(2,2-dimethyl-propionyloxy)-phenyl]-3,5-dioxo-hepta-1,6-dienyl}-phenyl ester, 5d. Bisdemethoxycurcumin, 5a was treated with pivaloyl chloride/TEA to give a yellow powder. R_{f}=0.35; ESI-MS m/z 477 (MH⁺), 475 (MH⁻); ¹HNMR δ 7.70 (d, J=18.0 Hz, 2H, Ph-CH—), 7.6 (m, 4H, Ph), 7.17 (m, 4H, Ph), 6.60 (d, J=18.0 Hz, 2H, —CH—CO—), 5.87 (s, 1H, —CH—), 1.22 (s, 18H, 2×(CH₃)₃).

5-Hydroxy-1,7-bis-(3-hydroxy-phenyl)-hepta-1,4,6-trien-3-one, 5e was prepared as described for 5a to give an orange powder. R_{f}=0.53; ESI-MS m/z 309 (MH⁺), 331 (MNa⁺), 307 (MH⁻); ¹HNMR δ 7.50 (d, J=15.9 Hz, 2H, Ph-CH), 7.16 (m, 2H, Ph), 7.00-6.95 (m, 4H, Ph), 6.78 (m, 2H, Ph), 6.53 (d, J=15.9 Hz, 2H, —CH—CO—), 5.80 (s, 1H, —CH—).

1,7-Bis-(4-dimethylamino-phenyl)-5-hydroxy-hepta-1,4,6-trien-3-one, 5f was prepared as described for 5a to give a deep orange powder. R_{f}=0.50; ESI-MS gave C₁₁H₁₂NO⁺ m/z 174 as major peak. ¹HNMR δ 7.60 (d, J=15.6 Hz, 2H, Ph-CH—), 7.45 (m, 4H, Ph), 6.68 (m, 4H, Ph), 6.42 (d, J=15.6 Hz, 2H, —CH—CO—), 5.73 (s, 1H, —CH—), 3.03 (s, 12H, 4×CH₃).

5-Hydroxy-1,7-bis-(3-hydroxy-4-methoxy-phenyl)-hepta-1,4,6-trien-3-one, 5g was prepared as described for 5a to give an orange powder. R_{f}=0.43; MP=182.8° C.; ESI-MS m/z 369 (MH⁺), 367 (MH⁻); ¹HNMR δ 7.40 (d, J=17.1 Hz; 2H, Ph-CH—), 6.99 (m, 2H, Ph), 6.91 (m, 2H, Ph), 6.73 (m, 2H, Ph), 6.34 (d, J=17.1 Hz, 2H, —CH—), 5.70 (s, 1H, —CO—CH—CO—), 3.78 (s, 6H, 2×CH₃).

5-Hydroxy-1,7-bis-(4-hydroxy-3-methoxy-phenyl)-hepta-1,4,6-trien-3-one, 5h was prepared as described for 5a to give an orange powder. R_{f}=0.35; ESI-MS m/z 369 (MH⁺), 367 (MH⁻); ¹HNMR δ 7.41 (d, J=15.6 Hz; 2H, Ph-CH—), 6.92 (m, 4H. Ph), 6.72 (m, 2H, Ph), 6.34 (d J=15.6 Hz, 2H, —CH—), 5.69 (s, 1H, —CO—CH—CO—), 3.77 (s, 6H, 2×CH₃).

5-Hydroxy-1,7-bis-(4-hydroxy-2-methoxy-phenyl)-hepta-1,4,6-trien-3-one, 5i was prepared as described for 5a to give orange powder. R_{f}=0.33; ESI-MS m/z 367 (MH⁻); ¹HNMR δ 7.87 (d, J=16.2 Hz; 2H, Ph-CH—), 7.40 (m, 2H, Ph), 6.77 (m, 2H, Ph), 6.63 (d, J=17.1 Hz, 2H, —CH—), 6.46 (m, 2H, Ph), 5.80 (s, 1H, —CO—CH—CO—), 3.85 (s, 6H, 2×CH₃).

5-Hydroxy-1,7-bis-(2-hydroxy-4-methoxy-phenyl)-hepta-1,4,6-trien-3-one, 5j was prepared as described for 5a to give a yellow powder, R_{f}=0.30; ESI-MS m/z 368 (M⁺). ¹HNMR δ 8.08 (s, 2H), 7.05 (m, 4H), 6.38-6.30 (m, 4H), 3.80 (s, 6H, 2×CH₃).

1,7-Bis-(3-chloro-4-hydroxy-phenyl)-5-Hydroxy-hepta-1,4,6-trien-3-one, 5k was prepared as described for 5a to give a yellow powder. R_{f}=0.14; ESI-MS m/z 376 (100%), 378 (66%), 377 (21%) (M⁺), 375 (100%), 377 (66%), 376 (21%) (MH⁻); ¹HNMR δ 7.54 (d, J=15.9 Hz; 2H, Ph-CH—), 7.40-7.36 (m, 4H, Ph), 6.33 (s, 2H, Ph), 6.47 (d, J=15.9 Hz, 2H, —CH—), 5.77 (s, 1H, —CO—CH—CO—).

5-Hydroxy-1,7-bis-(2-methoxy-phenyl)-hepta-1,4,6-trien-3-one, 5l was prepared as described for 5a to give a yellow powder. R_{f}=0.38; ESI-MS m/z 337 (MH⁺). ¹HNMR δ 8.05 (d, J=15.0 Hz, 2H, Ph-CH—), 7.85-7.60 (m, 4H, Ph), 7.11-6.70 (m, 4H, Ph), 6.66 (d, J=15.0 Hz, 2H, —CH—) 6.0 (s, 1H), 3.90 (s, 6H, 2×CH₃).

1,7-Bis-(5-fluoro-2-methoxy-phenyl)-5-Hydroxy-hepta-1,4,6-trien-3-one, 5m was prepared as described for 5a to give a yellow powder. R_{f}=0.26; ESI-MS m/z 371 (MH⁻); ¹HNMR δ 7.93 (d, J=15.0 Hz; 2H, Ph-CH—), 7.40 (m, 2H, Ph), 7.10-6.98 (m, 3H, Ph), 6.87-6.84 (m, 3H, Ph), 6.66 (d, J=15.0 Hz, 2H, —CH—), 5.87 (s, 1H, —CO—CH—CO—), 3.88 (s, 6H, 2×CH₃).

Synthesis of Curcumins.

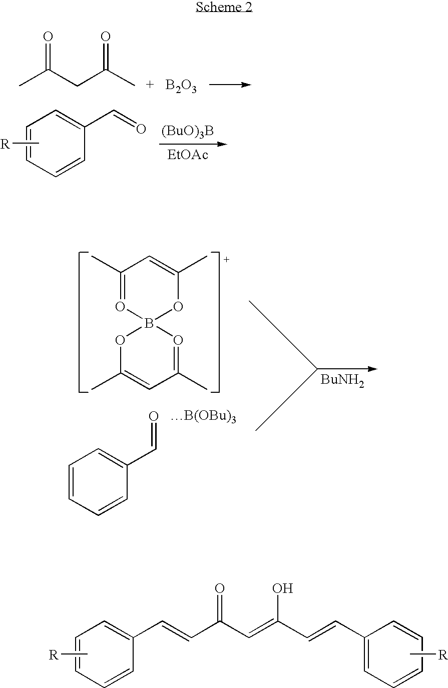

A substituted benzaldehyde (2 mmol) and tributyl Borate (4 mmol) was dissolved in 1 mL dry EtOAc. To a one dram vial was added boric anhydride (0.7 mmol) and acetylacetone (1 mmol) dissolved in 45 μL of dry EtOAc. After stirring for 1 h each at room temperature, the contents were combined. Four portions of butylamine totaling 0.2 mmol were added dropwise every 10 min. After 4 h, stirring was discontinued and the solution was left to sit overnight. The mixture was heated in an oil bath (50-60° C.) and quenched with HCl (1.5 mL of 0.4N). The solution was stirred for 1 h. The organic and aqueous layers were separated, the aqueous layer was extracted with EtOAc and the organic layers were combined and concentrated, dissolved in MeOH (500 μL), chilled overnight (4° C.), filtered, and rinsed with cold MeOH. The solid thus obtained was the highly purified curcumin.

(1E,4Z,6E)-5-hydroxy-1,7-diphenylhepta-1,4,6-trien-3-one (6). The general procedure above was followed to give a yellow solid (¹H NMR 300 MHz CDCl₃) δ 5.86s, 1H), δ 6.64 (d, J=15.9, 2H), δ 7.4 (m, 6H), δ 7.57 (m, 4H), δ 7.67 (d, J=15.9, 2H), MS (ESI) (Neg. ion) calcd for C₁₉H₁₆O₂ [M-H] 275.34. found 275.27; TLC EtOAc/Hexane 1:9 Rf=0.54.

1E,4Z,6E)-5-hydroxy-1,7-dip-tolylhepta-1,4,6-trien-3-one (7). The general procedure above was followed to give a yellow solid (¹H NMR 300 MHz CDCl₃) δ 2.39s, 6H), δ 5.83 (s, 1H), δ 6.60 (d, J=16.4, 2H), δ 7.21 (d, J=7.8, 4H), δ 7.27 (s, 1H), δ 4.65 (d, J=7.84H), δ 7.64 (d, J=15.7, 2H), MS (ESI) (Neg. ion) calcd for C₂₁H₂₀O₂ [M-H] 303.39. found 303.13, TLC EtOAc/Hexane 1:9 Rf=0.64.

1E,4Z,6E)-1,7-bis(3-fluorophenyl)-5-hydroxyhepta-1,4,6-trien-3-one (8). The general procedure above was followed to give a yellow solid.

1E,4Z,6E)-1,7-bis(4-thiolmethylphenyl)-5-hydroxyhepta-1,4,6-trien-3-one (9). The general procedure was followed to give an orange solid. (¹H NMR 300 MHz CDCl₃) δ 2.51s, 6H), δ 6.14s, 1H), δ 6.90 (d, J=15.9, 2H), δ 7.30 (d, J=8.5, 4H), δ 7.60 (d, J=15.9, 2H), δ 7.67 (d, J=8.5, 4H). Rf=0.37 (1:19 EOAc/Hexane).

(1E,4Z,6E)-1,7-bis(4-tert-butylphenyl)-5-hydroxyhepta-1,4,6-trien-3-one (10). The general curcumin synthesis procedure was followed to give a bright yellow solid (¹H 300 MHz CDCl₃) δ1.34s, 18H), δ 5.85 (s, 1H), δ 6.60 (d, J=16.15, 2H), δ 7.46 (q, J=17.6, 5.28H), δ 7.65 (d, J=15.9, 2H) MS (ESI) (Neg. ion) calcd for C₂₇H₃₂O₂ [M-H] 387.55. found 387.20; TLC EtOAc/Hexane 1:9 Rf=0.51.

### Example 10

Mgat3 and TLR transcription in cells from AD and control patients. We tested by qPCR Mgat3 and/0or TLR transcription in cells from AD patients and compared the results to those obtained from age-matched controls (Table 2). As discussed above, activation or up-regulation of macrophage Mgat3 or TLR's results in many functional outcomes, including the enhancement of amyloidosis and removal of Aβ, increased apoptosis, secretion of inflammatory cytokines, and other anti-AD antimicrobial activities. PBMC's from AD patients generally possess down-regulated Mgat3 and TLRs, whereas control PBMC's had up-regulated Mgat3 and TLRs. Thus, the ratio of Mgat3 or TLR transcription upon Aβ stimulation of AD versus control PBMCs provides an indicator and sensitive method to test the in vitro efficacy of drug candidates. Compounds with Mgat3 or TLR elevated transcription ratios are predicted to possess promise as anti-AD (and other neurodegenerative) diseases. Repeat assays with bisdemethoxy curcumin showed relative Mgat3 ratios of 3-5-fold. Ratios of greater than 1.0-2.0 suggest that the compounds up-regulate Mgat3 and TLRs and hold promise for use in drug development of anti-AD agents. The relative biological activity of 5a-m and 6-10 was ascertained in the in vitro Aβ assay described above (see Examples 4 & 9). The results are shown in Table 2 below.

TABLE 2
Relative ratio of Mgat3 transcription in AD/Mgat3 transcription
in control cells

| Compound | Relative Activity |
|---|---|
| 5a | ++ |
| 5b | − |
| 5c | − |
| 5d | − |
| 5e | + |
| 5f | ++++ |
| 5g | + |
| 5h | + |
| 5i | ++ |
| 5j | − |
| 5k | − |
| 5l | ++++ |
| 5m | +++++ |
| 6 | + |
| 7 | ++ |
| 8 | + |
| 9 | ++++ |
| 10 | + |

Dash (−) indicates minimal effect;
(+) indicates a ratio of 1.0-1.5;
(++) indicates a ratio of 1.5-2.0;
(+++ to ++++) indicates a ratio of 2.0 and above.

It is understood that the examples and embodiments described herein are for illustrative purposes only and that various modifications or changes in light thereof will be apparent to persons skilled in the art and are to be included within the spirit and purview of this application and scope of the appended claims. All publications, patents, and patent applications cited herein are hereby incorporated by reference in their entireties for all purposes.

What is claimed:

1. A method for treatment of Alzheimer disease comprising administering to a subject in need of such treatment a compound having the formula (I): 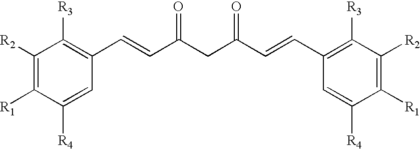
wherein
R₁, R₂, R₃ and R₄ are independently selected from the group consisting of hydrogen, (C1-C6)alkyl, (C1-C6)alkenyl, (C1-C6)alkynyl, heteroalkyl, halo (e.g., fluoro, chloro, bromo, iodo), (C1-C6)alkoxy, amino, (C1-C6)alkylamino, hydroxy, cyano, nitro, 5- or 6-member unsaturated, partially unsaturated or saturated heterocyclyl or carbocyclyl substituted with hydrido, acyl, halo, lower acyl, lower haloalkyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, alkyl sulfinyl and aryl, lower aralkylthio, lower alkylsulfinyl, lower alkylsulfonyl, aminosulfonyl, lower N-arylaminosulfonyl, lower arylsulfonyl, lower N-alkyl-N-arylaminosulfonyl; aryl selected from the group consisting of phenyl, biphenyl, naphthyl, and 5- and 6-membered heteroaryl optionally substituted with one, two, or three substituents selected from halo, hydroxyl, amino, nitro, cyano, carbamoyl, lower alkyl, lower alkenyloxy, lower alkoxy, lower alkylthio, lower alkylsulfinyl, lower alkylsulfonyl, lower alkylamino, lower dialkylamino, lower haloalkyl, lower alkoxycarbonyl, lower N-alkylcarbamoyl, lower N,N-dialkylcarbamoyl, lower alkanoylamino, lower cyanoalkoxy, lower carbamoylalkoxy, and lower carbonylalkoxy; and wherein further the acyl group is optionally substituted with a substituent selected from hydrido, alkyl, halo, and alkoxy.

2. The method of claim 1, wherein R₁, R₂, R₃, and R₄ is independently aryl having one or two ring hydrogens substituted with substituents selected from Cl, Br, I, —OR₄, —R₅, —OC(O)R₆, OC(O)NR₇R₈, —C(O)R₉, —CN, —NR₁₀R₁₁, —SR₁₂, —S(O)R₁₁, —S(O)₂R₁₄, —C(O)OR₁₅, —S(O)₂NR₁₆R₁₇; —R₁₈NR₁₉R₂₀ wherein R₄, R₅, R₆, R₇, R₈, R₉, R₁₀, R₁₁, R₁₂, R₁₃, R₁₄, R₁₅, R₁₆, R₁₇, R₁₈, R₁₉, and R₂₀ are the same or different and are branched or unbranched alkyl groups from one to eight carbon atoms or hydrogen radicals.

3. The method of claim 1, wherein R₁, R₂, R₃, and R₄ are each hydrogen.

4. The method of claim 1, wherein R₁, R₂, R₃, and R₄ are each an optionally substituted 5-membered carbocyclic ring.

5. The method of claim 1, wherein R₁, R₂, R₃, and R₄ are each an optionally substituted 5-membered heterocyclic ring having one or two heteroatoms selected from the group consisting of O, N or S.

6. The method of claim 1, wherein R₁, R₂, R₃, and R₄ are each an optionally substituted 6-membered carbocyclic ring.

7. The method of claim 1, wherein R₁, R₂, R₃, and R₄ are each an optionally substituted 6-membered heterocyclic ring having one or two heteroatoms selected from the group consisting of O, N or S.

8. A compound selected from the group consisting of 2,2-dimethyl-propionic acid 4-{7-[4-(2,2-dimethyl-propionyloxy)-phenyl]-3,5-dioxo-hepta-1,6-dienyl}-phenyl ester; 1,7-bis-(3-chloro-4-hydroxy-phenyl)-5-Hydroxy-hepta-1,4,6-trien-3-one; 1,7-bis-(5-fluoro-2-methoxy-phenyl)-5-Hydroxy-hepta-1,4,6-trien-3-one; and (1E,4Z,6E)-1,7-bis(4-tert-butylphenyl)-5-hydroxyhepta-1,4,6-trien-3-one, wherein said compound is useful for treatment of Alzheimer disease.

9. The compound of claim 8 which is 2,2-dimethyl-propionic acid 4-{7-[4-(2,2-dimethyl-propionyloxy)-phenyl]-3,5-dioxo-hepta-1,6-dienyl}-phenyl ester.

10. The compound of claim 8 which is 1,7-bis-(3-chloro-4-hydroxy-phenyl)-5-Hydroxy-hepta-1,4,6-trien-3-one.

11. The compound of claim 8 which is 1,7-bis-(5-fluoro-2-methoxy-phenyl)-5-Hydroxy-hepta-1,4,6-trien-3-one.

12. The compound of claim 8 which is (1E,4Z,6E)-1,7-bis(4-tert-butylphenyl)-5-hydroxyhepta-1,4,6-trien-3-one.

13. A method for in vitro screening of a compound for biological or pharmacological activity related to Alzheimer disease comprising the steps of:
(a) incubating a cell with the compound; and
(b) detecting the amount of amyloid-β (1-42) (Aβ) or other amyloid taken up, neutralized, consumed, or phagocytized as an indication of biological or pharmacological activity of the compound.

14. The method of claim 13, wherein the cell is an innate immune cell, monocyte, or macrophage, and wherein the cell being involved in the clearance of Aβ-plaques in vitro.

15. The method of claim 13, wherein the compound is a crude mixture of curcuminoids.

16. The method of claim 15, wherein the compound is a highly purified curcuminoid.

17. The method of claim 15, wherein the compound is a highly purified synthetic analog of a curcuminoid.

18. A method for predicting an efficacy of a drug in an individual, wherein said drug is an Mgat3 and/or TLR modulator (inducer) and said individual is suffering from or at risk of developing a CNS disorder related to Alzheimer disease amenable to treatment with the drug, said method comprising:
(a) isolating a biological sample from an individual, said biological sample comprising at least one of:
(i) a nucleic acid; and
(ii) a Mgat3 protein or TLR protein; and
(b) analyzing the biological sample to determine the presence or absence of the WT or other alleles of the Mgat3 gene in the individual, wherein the presence of WT Mgat3 is indicative of a positive clinical outcome for treatment of the disorder with the drug.

19. The method of claim 18, wherein the drug has a curcuminoid-like center.

20. The method of claim 19, wherein the drug is curcumin or a curcumin analog.

21. The method of claim 18, wherein the biological sample comprises a nucleic acid.

22. The method of claim 18, wherein the analyzing step comprises analyzing the nucleic acid from the biological sample to determine the nucleotide present at the Mgat3 and/or TLR gene coding region.

23. The method of claim 22, wherein the analyzing step comprises hybridization of nucleic acid from the biological sample with a nucleic acid selected from the group consisting of:
(a) a nucleic acid comprising at least 10 to 100 contiguous nucleotides of the nucleotide sequence set forth in SEQ ID NO:1 comprising at least:
(i) one of the nucleotides at key allelic positions; and
(ii) a base adjacent thereto; and
(b) a nucleic acid that is fully complementary to the nucleic acid of (a).

24. The nucleic acid of claim 23, wherein said nucleic acid is conjugated to a detectable marker.

25. The method of claim 18, further comprising determining the Mgat3 and/or TLR genotype at various nucleotide positions of the Mgat3 and/or TLR gene coding region.

26. A method for predicting an efficacy of a candidate agent for the treatment of a CNS disorder related to Alzheimer disease, wherein said candidate agent is a derivative of a predetermined therapeutic agent for the treatment of the disorder, said method comprising:
(a) contacting a sample of the Mgat3 or TLR protein from an AD individual with the candidate agent;
(b) contacting a sample of the Mgat3 or TLR protein from a healthy individual with the predetermined therapeutic agent; wherein said contacting occurs under conditions suitable for affording Mgat3 and/or TLR enzyme functional activity;
(c) determining for each of the samples the level of Mgat3 and/or TLR enzyme activity; and
(d) comparing the level of Mgat3 and/or TLR enzyme activity in the sample from the AD individual with the level of Mgat3 and/or TLR enzyme activity in the sample from the healthy individual;
wherein a greater level of Mgat3 and/or TLR enzyme activity in the sample from the AD individual relatively to the Mgat3 and/or TLR enzyme activity in the sample from the healthy individual is indicative of the efficacy of the candidate agent.

27. The method of claim 26, wherein the Mgat3 protein is a variant of Mgat3.

28. The method of claim 26, wherein the TLR protein is a variant of TLR.

29. The method of claim 26, wherein the predetermined therapeutic agent is curcumin or a related compound.

30. The method of claim 26, wherein the candidate agent has been modified to incorporate an Mgat3 and/or TLR inducer moiety.

31. The method of claim 30, wherein the Mgat3 and/or TLR inducer moiety is a curcuminoid-like center.

32. The method of claim 26, wherein determining the level of Mgat3 and/or TLR enzyme activity comprises detecting the level of an N-glycated peptide or protein as a function of the drug candidate in a sample.

33. A method for ex vivo treatment of a patient suffering from Alzheimer disease, the method comprising the steps of:
(a) obtaining a blood sample from the AD patient;
(b) contacting the blood sample with the compounds of formula (I); and
(c) injecting the modified blood sample into the patient.
